(19) United States
(12) Patent Application Publication
(10) Pub. No.: US 2025/0332234 A1
(43) Pub. Date: Oct. 30, 2025

(54) S100A9 VACCINES FOR CANCER AND ATHEROSCLEROSIS

(71) Applicant: The Regents of the University of California, Oakland, CA (US)

(72) Inventors: Nicole F. Steinmetz, San Diego, CA (US); Oscar A. Ortega-Rivera, San Diego, CA (US); Young Hun Chung, San Diego, CA (US)

(21) Appl. No.: 18/868,626

(22) PCT Filed: May 24, 2023
(86) PCT No.: PCT/US2023/023440

§ 371 Date: Nov. 22, 2024

Related U.S. Application Data
(60) Provisional application No. 63/345,676, filed on May 25, 2022.

(51) Int. Cl. A61K 39/00; A61P 37/04

(52) U.S. Cl. CPC A61K 39/0011; A61P 37/04; A61K 2039/5258

(57) ABSTRACT

Provided herein are compositions and methods that contain a nanoparticle and a peptide target (e.g., peptide 101-110 of S100A9) to treat cardiovascular disease and cancer.

### CROSS-REFERENCE TO RELATED APPLICATION

This application claims priority under 35 U.S.C. § 119 (e) to U.S. Provisional Application No. 63/345,676, filed May 25, 2022, the contents of which are incorporated herein by reference in their entireties.

### STATEMENT OF GOVERNMENT SUPPORT

This invention was made with government support under CA224605 and HL137674, awarded by the National Institutes of Health (NIH). The government has certain rights in the invention.

### BACKGROUND

Throughout this disclosure, patent and technical publications are referenced by an identifying citation or an Arabic numeral the full bibliographic citation for which is found immediately preceding the claims. The disclosures of these references are incorporated herein to more fully describe the state of the art to which this disclosure pertains.

Passive immunization (monoclonal antibody therapy) against pro-atherogenic cytokines such as IL-1β²⁰ and TNF-α²¹ was shown to reduce the risk of cardiovascular events. Furthermore, the US Food and Drug Administration recently approved the monoclonal antibodies alirocumab and evolocumab targeting the proprotein convertase subtilisin/kexin-9 (PCSK9), a cholesterol metabolism checkpoint protein^{22, 23, 24}. However, passive immunotherapy requires repeated dosing and lifelong treatment, which is too expensive for many patients²⁵. Thus, a need exists in the art for an active immunotherapy (vaccination) that is cost-effective that can achieve protective effects. This disclosure satisfies this need and provides related advantages as well.

### SUMMARY OF THE DISCLOSURE

In accordance with the above, an aspect of the disclosure is directed to a nanoparticle comprising a virus or virus like particle (VLP) and a S100A9 peptide epitope. In some embodiments, the virus is Cowpea mosaic virus (CPMV) and the virus like particle is Qβ capsid protein (CP).

In some embodiments, the S100A9 peptide epitope comprises or consists of 101 [PGHHHKPGLG] 110 (human) (SEQ ID NO: 1) or 101 [RGHGHSHGKG] 110 (murine) (SEQ ID NO: 2).

This disclosure provides virus-like particles delivering a vaccine targeting peptides 101 [PGHHHKPGLG]₁₁₀ (human) (SEQ ID NO: 1) or ₁₀₁[RGHGHSHGKG]₁₁₀ (murine) (SEQ ID NO: 2) that treat metastatic cancer and cardiovascular disease. One embodiment of this disclosure provides virus-like particles (VLPs) from bacteriophage Qβ to display a B-cell epitope from the mouse S100A9 protein ₁₀₁[PGHHHKPGLG]₁₁₀ (human) (SEQ ID NO: 1) or ₁₀₁[RGHGHSHGKG]₁₁₀ (murine) (SEQ ID NO: 2) for use in these applications.

In some embodiments, the virus or VLP has an exposed lysine side chain.

In some embodiments, the S100A9 epitope comprises a linker or a c-terminal cysteine, or optionally, is detectably labeled.

In some embodiments, a N-hydroxysuccinimide (NHS) ester conjugates with the lysine side chain and a maleimide of a maleimide-polyethylene glycol8 (SM(PEG)8) conjugates with the c-terminal cysteine of the peptide.

In some embodiments, the nanoparticle has an average diameter of from about 10 to about 50 nm.

In some embodiments, wherein the linker comprises the peptide GSG.

Another aspect of the disclosure is directed to a polynucleotide encoding the nanoparticle of the instant disclosure.

Another aspect of the disclosure is directed to a vector comprising the polynucleotide of the instant disclosure.

Another aspect of the disclosure is directed to an isolated host cell comprising the nanoparticle of the instant disclosure, or the polynucleotide of the instant disclosure, or the vector of the instant disclosure.

Another aspect of the disclosure is directed to a plurality of the nanoparticles of the instant disclosure, wherein the nanoparticles are the same or different from each other.

Another aspect of the disclosure is directed to a composition comprising the nanoparticle of the instant disclosure, or the plurality of nanoparticles of the instant disclosure, and a carrier. In some embodiments, the composition further comprises an additional therapeutic agent or an adjuvant.

Another aspect of the disclosure is directed to a method for inducing an immune response in a subject in need thereof comprising administering to the subject: the nanoparticle of the instant disclosure, the plurality of nanoparticles of the instant disclosure or the composition of the instant disclosure.

Another aspect of the disclosure is directed to a method for one or more of: inducing an immune response, treating cardiovascular disease and associated disorders, atherosclerosis, cancer, and associated disorders, administering to a subject in need thereof with the nanoparticle of the instant disclosure, the plurality of nanoparticles of the instant disclosure or the composition of the instant disclosure.

In some embodiments, the cancer is selected from melanoma, breast cancer, prostate cancer, lung cancer, ovarian cancer, skin cancer, bladder cancer, pancreatic cancer, gastric cancer, esophageal cancer, colon cancer, glioma, cervical cancer, hepatocellular cancer, or thyroid cancer.

In some embodiments, the cancer is a primary or a metastatic cancer.

In some embodiments, the cancer is metastatic or primary lung cancer, ovarian cancer, colon cancer or breast cancer.

In some embodiments, the cancer is metastatic melanoma or metastatic triple negative breast cancer.

In some embodiments, the cancer is metastatic cancer selected from intraperitoneal disseminated metastatic cancer, metastatic ovarian cancer, metastatic colon cancer, metastatic liver cancer, metastatic lung cancer.

In some embodiments, the subject is a mammal or a human.

In some embodiments, the cancer expresses S100A9.

In some embodiments, the method further comprises the administration of a different cancer therapy or tumor resection.

In some embodiments, the administering comprises intramuscular, sub-cutaneous, or intraperitoneal delivery.

In some embodiments, the disease is cancer and the treatment comprises one or more of: inhibiting metastatic potential of the cancer; recurrence prevention, reduction in tumor size; a reduction in tumor burden, longer progression free survival and longer overall survival of the subject.

In some embodiments, the disease is CVD and associated disorders, wherein the method further comprises administering one or more of statins to reduce plasma cholesterol levels, angioplasty, lifestyle changes, beta blockers, nitrates, angiotensin-converting enzyme inhibitors, angiotensin-2 receptor blockers, calcium channel blockers or diuretics.

Another aspect of the disclosure is directed to a kit comprising one or more of the nanoparticles of the instant disclosure, the plurality of nanoparticles of the instant disclosure or the composition of the instant disclosure and optional instructions for use.

### Cancer Applications

Metastasis remains one of the toughest challenges in cancer treatment today, and most cancer-related deaths are attributed to the development of meteor static sites in distal organs. A calcium-binding protein called S100A9 plays a major role in metastasis, growth, and aggressiveness in a multitude of cancers. Applicant discloses herein that a plant virus, the cowpea mosaic virus (CPMV), and a bacteriophage, QB, are an adjuvant and a carrier for the development of a subunit vaccine targeting S100A9. Applicant shows that the CPMV-S100A9 and QB-S100A9 vaccines elicit strong antibody titers specific not only to the peptide epitope, but also against the S100A9 full-length protein with no cross-reactivity to S100A8, another member of the S100A family. Vaccination of mice protects from intravenous tumor challenge with significant reduction of tumor nodules within the lungs of mice compared to controls in both melanoma and breast cancer metastatic models; vaccination also improves survival in a breast cancer surgical removal model. Efficacy is correlated with significantly reduced S100A9 levels within the lungs and blood compared to non-vaccinated mice, and that this leads to an increase in immunostimulatory cytokines and immune cells and a decrease in immunosuppressive cytokines and immune cells. Overall, it is demonstrated that vaccination against S100A9 with both CPMV and QB significantly reduces metastatic tumor burden within the lungs of mice through the elimination of S100A9.

Vaccination of mice protects from intravenous tumor challenge with significant reduction of tumor nodules within the lungs of mice compared to controls in both melanoma and breast cancer metastatic models; vaccination also improves survival in a breast cancer surgical removal model. Efficacy is correlated with significantly reduced S100A9 levels within the lungs and blood compared to non-vaccinated mice, and that this leads to an increase in immunostimulatory cytokines and immune cells and a decrease in immunosuppressive cytokines and immune cells. Overall, Applicant demonstrated that vaccination against S100A9 with both CPMV and Qβ significantly reduces metastatic tumor burden within the lungs of mice through the elimination of S100A9.

Thus, for cancer metastasis applications, Applicant shows that the CPMV-S100A9 and Qβ-S100A9 vaccines elicit strong antibody titers specific not only to the peptide epitope, but also against the S100A9 full-length protein with no cross-reactivity to S100A8, another member of the S100A family. Vaccination of mice protects from intravenous tumor challenge with significant reduction of tumor nodules within the lungs of mice compared to controls in both melanoma and breast cancer metastatic models; vaccination also improves survival in a breast cancer surgical removal model. Efficacy is correlated with significantly reduced S100A9 levels within the lungs and blood compared to non-vaccinated mice, and that this leads to an increase in immunostimulatory cytokines and immune cells and a decrease in immunosuppressive cytokines and immune cells. Overall, Applicant demonstrated that vaccination against S100A9 with both CPMV and Qβ significantly reduced metastatic tumor burden within the lungs of mice through the elimination of S100A9.

### Cardiovascular Disease

The standard therapy for cardiovascular disease (CVD) is the administration of statins to reduce plasma cholesterol levels, but this requires lifelong treatment. Applicant developed a CVD vaccine candidate that targets the pro-inflammatory mediator calprotectin by eliciting antibodies against the S100A9 protein. The vaccine, based on bacteriophage Qβ virus-like particles (VLPs) displaying S100A9 peptide epitopes, was formulated as a slow-release PLGA:VLP implant by hot-melt extrusion. The single-dose implant elicited S100A9-specific antibody titers comparable to a three-dose injection schedule with soluble VLPs. In an animal model of CVD (ApoE−/− mice fed on a high-fat diet), the implant reduced serum levels of calprotectin, IL-1β, IL-6 and MCP-1, resulting in less severe aortic lesions. This novel implant was therefore able to attenuate atherosclerosis over a sustained period and offers a novel and promising strategy to replace the repetitive administration of statins for the treatment of CVD.

In an animal model of CVD (ApoE−/− mice fed on a high-fat diet), the implant reduced serum levels of calprotectin, IL-1β, IL-6 and MCP-1, resulting in less severe aortic lesions. This novel S100A9 vaccine implant was therefore able to attenuate atherosclerosis over a sustained period and offers a novel and promising strategy to replace the repetitive administration of statins for the treatment of CVD. Applicant developed an anti-inflammatory vaccine targeting the S100A9 protein in order to reduce serum levels of calprotectin (S100A8/S100A9 heterodimer) that can be useful for the treatment of cardiovascular diseases (CVD) or cancer metastasis.

For CVD applications, the efficacy and safety of the vaccine was tested in a traditional prime-boost-boost schedule vs the single-dose slow-release injectable implant in healthy mice and in the ApoE−/− model of atherosclerosis by measuring antibody titers and immune responses, plasma levels of calprotectin, IL-1β, IL-6, and MCP-1, and the severity of aortic lesions in the aortic arch and thoracic aorta.

The Qβ-S100A9 vaccine can keep the plasma levels of calprotectin (S100A8/S100A9 heterodimer) at physiological levels vs control group (highly increased) after atherosclerosis induction with high-fat diet, and as mechanism of action (MOA) Applicant observed that this reduction in plasma levels of calprotectin was correlated with the reduction in plasma level of different pro-inflammatory cytokines and chemokines as well (IL-1β, IL-6, and MCP-1).

### BRIEF DESCRIPTION OF THE DRAWINGS

FIGS. 1A-1B: Similarity between the human and mouse S100A9 proteins. (FIG. 1A) Structural models (PDB 6DS2 and 6ZDY). The C-terminal target epitope (residues 101-110) and its amino acid sequence is indicated with an arrow. The structures were rendered using UCSF Chimera³⁷. (FIG. 1B) Alignment of the human (top, Uniprot ID P06702) and mouse (bottom, Uniprot ID P31725) S100A9 amino acid sequences, revealing 58% identity and 74% similarity. The boxes indicate fully conserved positions and chemically similar residues. The two metal-binding sites (S1 and S2) are shown as series of two and four arrows, respectively³⁸. The sequences were aligned using Clustal Omega³⁹.

FIGS. 2A-2F: Production and characterization of QβS100A9 VLPs. (FIG. 2A) Schematic (not to scale) of the unmodified Qβ capsid protein (CP) and the QβS100A9 fusion CP with S100A9 epitope ₁₀₁ [RGHGHSHGKG]₁₁₀ (SEQ ID NO: 2) and an intervening GSG linker, and a map of expression vector pCOLA_Qβ_QBS100A9 with both genes driven by independent T7 promoters and a single T7 terminator (lacI=lactose repressor gene, ColA ori=ColA origin of replication, KanR=kanamycin resistance gene). (FIG. 2B) SDS-PAGE analysis of unmodified Qβ (left) and QβS100A9 VLPs (right) under reducing conditions. Unmodified Qβ CP (CP˜14 kDa) and modified QβS100A9 (S100A9-CP˜15.2 kDa) were visualized after staining with GelCode Blue Safe protein stain. (FIG. 2C) The number of peptides displayed per hybrid QβS100A9 VLP was determined by densitometry. QβS100A9 VLPs were then used in two delivery formulations (soluble VLPs and slow-release implants). (FIG. 2D) DLS of unmodified Qβ and QβS100A9 VLPs shows monodisperse particles. Z-average (dm)=32.4±0.5 for unmodified Qβ and 39.2±0.2 for QβS100A9. PDI=0.11 for unmodified Qβ and 0.01 for QβS100A9. (FIG. 2E) FPLC analysis of unmodified Qβ and QβS100A9 VLPs display similar elution curves. (FIG. 2F) TEM images of negatively-stained unmodified Qβ and QβS100A9 VLPs (scale bar=100 nm). All samples were tested in triplicate (n=3) for all characterization methods, and the pictures and data shown are representatives of those triplicates.

FIGS. 3A-3H: Immunization of C57B16J mice with QβS100A9 vaccines. (FIG. 3A) Mice were immunized with 100 μg per injection of the soluble QβS100A9 vaccine or 5 μg of the free peptide (control) in a traditional prime plus two boosts schedule. (FIG. 3B) ELISA against S100A9 peptide from the soluble vaccine showing endpoint IgG titers. (FIG. 3C) ELISA against S100A9 peptide from the soluble vaccine showing absorbance of IgG subclasses and Ig isotypes over time. (FIG. 3D) T-cell helper (Th)-biased profile based on the IgG1/IgG2b ratio of the soluble vaccine. (FIG. 3E) Mice were immunized with a PLGA implant containing 300 μg QβS100A9 VLPs (equivalent to the total amount of soluble VLPs) or non-modified Qβ VLPs (control). (FIG. 3F) ELISA against S100A9 peptide from implant vaccine showing endpoint IgG titers. (FIG. 3G) ELISA against S100A9 peptide from implant vaccine showing absorbance of IgG subclasses and Ig isotypes over time. (FIG. 3H) T-cell helper (Th)-biased profile based on the IgG1/IgG2b ratio of the implant vaccine. Data are means±SEM (n=5 biological samples, unpaired two-tailed t-test with 95% confidence value, *p<0.05).

FIG. 4: Specificity of antibodies elicited against the S100A9 epitope displayed on VLPs or as a free peptide. Pooled plasma (dilution 1:100) from mice vaccinated with QβS100A9 VLPs (right column) or the free peptide (left column) was tested against mouse recombinant S100A8, S100A9 and heterodimer S100A8/9 (calprotectin) by dot blot (1 μg/dot). Plasma from mice vaccinated with QβS100A9 recognized the S100A9 protein and its heterodimer S100A8/9, but not the S100A8 protein. The results were similar for mice vaccinated with the free peptide, although the signals were weaker.

FIGS. 5A-5D: Plasma biomarkers and ELISpot assays for the analysis of vaccine safety. (FIGS. 5A-5C) ELISpot assays using splenocytes from mice vaccinated with (FIG. 5A) soluble QβS100A9 VLPs or (FIG. 5B) PLGA-based QβS100A9 implants compared to (FIG. 5C) splenocytes from naïve mice. The splenocytes from each vaccinated (n=3) or naïve (n=2) group were cultivated at least in duplicate with medium (negative control), free peptide, whole S100A9 protein, unmodified Qβ VLPs, or PMA/ionomycin (positive control). (FIG. 5D) Three plasma biomarkers related to liver injury (AST and ALT) and kidney injury (KIM-1) were evaluated at week 0 (baseline) and week 12 after vaccination (n=4 from pooled plasma). Dashed lines in the AST and ALT plots indicate the threshold for normal healthy mice. Data are means±SEM (unpaired two-tailed t-test with 95% confidence value, *p<0.05).

FIGS. 6A-6D: Immunogenicity of the QβS100A9 vaccine implant in a mouse model of atherosclerosis fed on a high-fat western diet. (FIG. 6A) Vaccination and diet schedule for ApoE^{-/-} male mice (n=10 per group). Two groups were tested, one vaccinated with the QβS100A9 implant (300 μg total dose) and a control group vaccinated with an equivalent dose of unmodified Qβ VLPs. (FIG. 6B) Anti-S100A9 peptide antibody titers at different time points after implantation. (FIG. 6C) IgG1/IgG2b ratios at four time points to characterize the Th profile (ratio<1=Th1-biased, ratio>1=Th2-biased). The Th profile shifted from Th1-biased to balanced Th1/Th2 over time. (FIG. 6D) Complete immunoglobulin profile over time. Data are means±SEM (n=10, unpaired two-tailed t-test with 95% confidence value, *p<0.0001).

FIGS. 7A-7G: Effect of QβS100A9 vaccine implants on a mouse model of atherosclerosis fed on a high-fat western diet. (FIG. 7A) Body weight (grams) determined weekly. (FIG. 7B) Total cholesterol in plasma determined at four time points. (FIG. 7C) The presence of atherosclerotic plaques shown as the percentage of lesion determined by oil red O staining. Representative aortic arch and thoracic aorta images from QβS100A vaccinated and control groups are shown, with vaccinated groups showing much fewer and smaller atherosclerotic plaques. (FIGS. 7D-7G) Levels of key biomarkers in plasma before (week 0) and after vaccination (weeks 8, 12 and 24): (FIG. 7D) calprotectin, (FIG. 7E) monocyte chemoattractant protein-1 (MCP-1), (FIG. 7F) interleukin-6 (IL-6), and (FIG. 7G) interleukin-1B (IL-1β). Data are means±SEM (n=10), unpaired two-tailed t-test, 95% confidence value, p<0.05 was considered the threshold for statistical significance vs control group.

FIG. 8: Images from the aortic arch and thoracic aorta of ApoE−/− mice, at week 24, from QβS100A9 vaccinated and control groups are shown, with the presence of atherosclerotic plaques lesion determined by oil red O staining. Quantitative data are shown in FIG. 7.

FIG. 9: Plasma calprotectin levels in healthy C57BL/6 mice. Pooled plasma from mice vaccinated with soluble QβS100A9 VLPs (n=5) vs free peptide (control, n=5) vs PLGA-based slow-release QβS100A9 VLP implants (n=5) vs Qβ VLP implants (control, n=3) were tested for calprotectin levels by ELISA at weeks 12 and 20. Data are means #SEM. Statistical analysis was carried out by applying an unpaired two-tailed t-test.

FIG. 10: Organ weights of ApoE^{-/-} mice at week 24. The organs were collected and weighed at the end of the experiment. Statistical analysis was carried out by applying an unpaired two-tailed t-test. No significant difference was observed between groups.

FIGS. 11A-11B: S100A9 peptide epitope and its conjugation to CPMV and Qβ. (FIG. 11A) Structure of S100A9 and peptide epitope sequence of the S100A9 epitope in mice compared to humans. The identical sequences are underlined. (FIG. 11B) CPMV is produced through mechanical inoculation of black-eyed pea No. 5 plants while Qβ VLPs are expressed in E. coli. An SM(PEG)₈ linker is conjugated to lysines (shown by black spheres) on the exterior of the viral capsids followed by maleimide coupling of the cysteine-terminating S100A9 peptide. The added CGSG linker is underlined. Qβ contains more surface exposed Lys (720 vs 300 for CPMV), which allows for greater peptide conjugation. The figures were drawn on Biorender.com. The structures of the mouse and human S100A9, CPMV, and Qβ were created on Chimera (mouse S100A9 PDB ID: 6DS2, human S100A9 PDB ID: 6ZDY, CPMV PDB ID: 1NY7, Qβ PDB ID: 1QBE), and the SM(PEG)₈ chemical structure was drawn on ChemDraw. The small (5-fold axis) and large (3- and 2-fold axis) CP of CPMV are shown, and for Qβ CPs are pictured according to their symmetry (5-3-2 fold axis, respectively). CPMV has pseutdo-T3 and Qβ has T3 symmetry.

FIGS. 12A-12E: Antibody titers against the S100A9 epitope following vaccination of mice. (FIG. 12A) Vaccine injection schedule in both C57BL/6J (left) and BALB/C (right) mice. The vaccine candidates were given in a prime and double boost spaced two weeks apart. Tumor burden is shown in FIG. 13. (FIG. 12B) Titers against the S100A9 peptide in C57BL/6J mice (n=3-4). The endpoint titers were determined as the titer range at which the absorbance was twice the absorbance of the blank. (FIG. 12C) IgG Isotyping of the CPMV and Qβ-S100A9 vaccines in C57BL/6J mice. (FIG. 12D) Titer production and endpoint titers against the S100A9 peptide in BALB/C mice (n=2-3). (FIG. 12E) IgG isotyping of the Qβ-S100A9 vaccines in BALB/C mice. The injection schedule schematics were created on Biorender.com. The error bars represent the standard deviation.

FIGS. 13A-13G: Reduction of B16F10 and 4T1-Luc tumor nodules following vaccination. (FIG. 13A) Injection schedule in C57BL/6J mice. Lungs were harvested after 2 or 3 weeks depending on the number of cells injected. (FIG. 13B) Quantitative analysis of B16F10 tumor nodules within the lungs (n=4-10). Error bars represent the standard deviation. (FIG. 13C) Qualitative images of the tumor nodules in FIG. 13B). The images are representative images of lungs from each group. The black dots on the lungs represent the B16F10 tumor nodules. (FIG. 13D) Injection schedule in BALB/C mice. (FIG. 13E) Quantitative analysis of the 4T1-Luc tumor nodules within the lungs (n=4-10). Error bars represent the standard deviation. (FIG. 13F) Representative images of lungs from FIG. 13E). The red arrows are pointing towards 4T1-Luc tumor nodules. The lungs are yellow due to the storing of the lungs in Bouin's solution to visualize 4T1-Luc tumor nodules. (FIG. 13G) Lung luminescence of mice from ROI measurements taken on the IVIS. Error bars represent the standard deviation. The injection schedule schematics were created on Biorender.com. All analyses were done on GraphPad Prism, and the tumor nodules were compared using one-way ANOVA. *=p<0.05, **=p<0.01, ****=p<0.0001, ns=not significant.

FIGS. 14A-14C: Metastasis study of surgically removed 4T1-Luc orthotopic tumors. (FIG. 14A) Injection schedule. The primary 4T1-Luc tumors were injected s.c. followed by surgical removal after two weeks. The lungs of the mice were then imaged every two days for onset of metastases. (FIG. 14B) Luminescence of the 4T1-Luc tumors within the lungs of mice as measured by ROI measurements on the IVIS. The survival of the mice is also shown. In the tumor growth curves, the luminescence is shown on the y-axis is log scale (n=5-10). (FIG. 14C) IVIS imaging of 4T1-Luc metastasis within the lungs of mice. The colored regions represent areas of tumor growth. The injection schedule schematics were created on Biorender.com.

FIGS. 15A-15H: S100A8/9 levels within the lungs and sera of B16F10- and 4T1-Luc-inoculated mice as a function of Qβ-S100A9 vaccination. (FIG. 15A) Injection and lung/sera collection schedule. (FIG. 15B) S100A8/9 levels in the lungs of C57BL/6J mice. (FIG. 15C) S100A8/9 levels in the sera of C57BL/6J mice. (FIG. 15D) S100A8/9 levels in the lungs of BALB/C mice. (FIG. 15E) S100A8/9 levels in the sera of BALB/C mice. (FIG. 15F) Scatter plot of mice comparing tumor nodule formation and S100A8/9 concentration within the sera. A line of best fit was plotted with an R² of 0.9025. (FIG. 15G) Tumor nodules within the lungs of B16F10-inoculated mice. The red arrows are pointing towards areas of tumor nodule formation. (FIG. 15H) Tumor nodules within the lungs of 4T1-Luc-inoculated mice. The arrows are pointing towards areas of tumor nodule formation. All experiments were done with n=2-3, and the error bars represent the standard deviation. All statistics were accomplished with Student's T test. The injection schedule schematics were created on Biorender.com. *=p<0.05, **=p<0.01, ***=p<0.001, ns=not significant.

FIGS. 16A-16E: Analysis of cytokine and MDSC levels within the lungs of B16F10- and 4T1-Luc-inoculated mice after Qβ-S100A9 vaccination. (FIG. 16A) Injection and lung harvesting schedule. (FIG. 16B) Concentration of IL-10, TGFβ, IL-6, IFNγ, and IL-12 in the lungs of vaccinated and naïve C57BL/6J mice inoculated with B16F10 metastatic tumors. (FIG. 16C) Concentration of IL-10, TGFβ, IL-6, IFNγ, and IL-12 in the lungs of vaccinated and naïve BALB/C mice inoculated with 4T1-Luc metastatic tumors. #IL-6 is listed as immunosuppressive, but also contributes to an immunostimulatory response. (FIG. 16D) M-MDSC and G-MDSC populations within the lungs of vaccinated and unvaccinated C57BL/6J mice inoculated with B16F10 tumors. (FIG. 16E) M-MDSC and G-MDSC populations within the lungs of vaccinated and unvaccinated BALB/C mice inoculated with 4T1-Luc tumors. All experiments were done with n=2-4, and the analyses were run using Student's T-test. The error bars represent the standard deviation. *=p<0.05, **=p<0.01, ***=p<0.001, ****=p<0.0001, ns=not significant. The injection schedule schematics were created on Biorender.com.

FIGS. 17A-17F: Characterization of CPMV-S100A9 and Qβ-S100A9 nanoparticles. (FIG. 17A) UV-VIS spectra of CPMV-S100A9. The inset indicates the absorbance at 260 and 280 nm as well as the absorbance ratio (A260/A280). (FIG. 17B) Agarose gel electrophoresis of CPMV-S100A9 and Qβ-S100A9 particles. The left gels are the nucleic acid stains while the right gels are protein stains. Qβ encapsulates host RNA during capsid formation, which can be stained. (FIG. 17C) SDS-PAGE of CPMV-S100A9 and Qβ-S100A9. The appearance of slower mobility bands above the CPs of CPMV-S100A9 and Qβ-S100A9 demonstrate successful conjugation of peptides to the CPs. S CP=small coat protein, L CP=large coat protein. (FIG. 17D) TEM of CPMV-S100A9 and Qβ-S100A9. (FIG. 17E) DLS of CPMV-S100A9 and Qβ-S100A9. The inset shows the measured average size of the particles as well as the PDI. (FIG. 17F) SEC elution profiles of CPMV-S100A9 and Qβ-S100A9. The inset shows the elution peak of the particles as well as the absorbance ratio of 260 to 280 nm at that peak.

FIGS. 18A-18C: Characterization of WT CPMV and Qβ particles. (FIG. 18A) TEM. The scale bar represents 100 nm. (FIG. 18B) DLS. The inset is showing the measured size as well as the polydispersity index of the particles. (FIG. 18C) FPLC. The inset is showcasing the elution peak of the particles through an SEC as well as the ratio of the absorbances at 260 and 280 nm at the elution peak.

FIG. 19: Titers against the S100A9 peptide in BALB/C mice vaccinated with CPMV-S100A9. CPMV-S100A9 was unable to generate any titers by week 4 in BALB/C mice (n=2-3).

FIGS. 20A-20B: Complete isotyping of the CPMV and Qβ-S1009 vaccines. (FIG. 20A) Isotyping of the sera in C57BL/6J mice injected with CPMV and Qβ-S100A9. (FIG. 20B) Isotyping of the sera in BALB/C mice injected with Qβ-S100A9. All samples were run in duplicate or triplicate, and the error bars represent the standard deviation.

FIGS. 21A-21C: Generation of antibodies against Qβ in both C57BL/6J and BALB/C mice. (FIG. 21A) Injection and bleeding schedule. (FIG. 21B) Antibody titers against the Qβ coat protein in C57BL/6J mice (n=3-4) injected with Qβ, Qβ-S100A9, and S100A9. (FIG. 21C) Antibody titers against the Qβ coat protein in BALB/C mice (n=2-3) injected with Qβ, Qβ-S100A9, and S100A9. The injection schedule schematic was created on Biorender.com. The error bars represent the standard deviation.

FIGS. 22A-22B: Generation of antibodies against CPMV in C57BL/6J mice. (FIG. 22A) Injection and bleeding schedule. (FIG. 22B) Antibody titers against the CPMV coat protein in mice (n=3-4) injected with CPMV, CPMV-S100A9, and S100A9. The CPMV-S100A9 vaccine was not tested in BALB/C mice, as CPMV-S100A9 was unable to produce titers against the S100A9 peptide in BALB/C mice (FIG. 18). The injection schedule schematic was created on Biorender.com. The error bars represent the standard deviation.

FIGS. 23A-23C: WBs and DBs against full-length S100A8 and S100A9. (FIG. 23A) WBs against full-length S100A8 and S100A9 using the sera from C57BL/6J mice injected with CPMV-S100A9, Qβ-S100A9, and S100A9. The red boxes are highlighting the signals produced during the WBs. The blots show that the antibodies produced following vaccination can bind to full-length S100A9, but not S100A8, a close family member to S100A9. (FIG. 23B) DBs against full-length S100A8 using the sera from C57BL/6J mice. For the DBs, the sera from the groups of each vaccinated mice were pooled. There is no signal in any of the DBs indicating that the antibodies produced during vaccination do not bind to S100A8. (FIG. 23C) DBs against full-length S100A9 using pooled sera from C57BL/6J mice. The signal from Qβ-S100A9 and CPMV-S100A9 indicate that the antibodies produced can bind to full-length, native S100A9.

FIG. 24: Weight measurements from the BALB/C mice used in FIG. 14. The weight of the mice in the Qβ and PBS mice fell continuously until the last measurement. The data is only shown until day 14 as the lungs were harvested on day 14 for tumor nodule counting. The error bars represent the standard deviation (n=4-10).

FIGS. 25A-25B: Complete ELISAs of the S100A8/9 measurements within the lungs of mice. (FIG. 25A) Injection and lung harvesting schedule. (FIG. 25B) ELISA measurements of S100A8/9 within the lungs of vaccinated and unvaccinated C57BL6/J and BALB/C mice (n=3) using an S100A8/9 detection kit (R&D Systems). The error bars represent the standard deviation. The injection schedule schematic was created on Biorender.com.

FIGS. 26A-26B: Complete ELISAs of the S100A8/9 measurements in the blood of mice. (FIG. 26A) Injection and blood collection schedule. (FIG. 26B) ELISA measurements of S100A8/9 within the blood of vaccinated and unvaccinated C57BL6/J and BALB/C mice (n=3) using an S100A8/9 detection kit (R&D Systems). The error bars represent the standard deviation. The injection schedule schematic was created on Biorender.com.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the disclosure. All publications, patent applications, patents and other references mentioned herein are incorporated by reference in their entirety.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications are referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the disclosure and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human,

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the nanoparticle of this disclosure and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

As used herein, the phrase “immune response” or its equivalent “immunological response” refers to the development of a cell-mediated response (e.g. mediated by antigen-specific T cells or their secretion products). A cellular immune response is elicited by the presentation of polypeptide epitopes in association with Class I or Class II MHC molecules, to treat or prevent a viral infection, expand antigen-specific B-reg cells, TC1, CD4+T helper cells and/or CD8+ cytotoxic T cells and/or disease generated, autoregulatory T cell and B cell “memory” cells. The response may also involve activation of other components. In some aspect, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells-non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

The term “immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small secreted proteins released by immune cells that have a specific effect on the interactions and communications between the immune cells. Cytokines can be pro-inflammatory or anti-inflammatory. Non-limiting example of a cytokine is Granulocyte-macrophage colony-stimulating factor (GM-CSF), which stimulates stem cells to produce granulocytes (neutrophils, eosinophils, and basophils) and monocytes.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

An “effective amount” or “efficacious amount” refers to the amount of an agent or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated. In some embodiments the effective amount will depend on the size and nature of the application in question. It will also depend on the nature and sensitivity of the target subject and the methods in use. The skilled artisan will be able to determine the effective amount based on these and other considerations. The effective amount may comprise, or alternatively consist essentially of, or yet further consist of one or more administrations of a composition depending on the embodiment.

Cardiovascular disease (CVD) is a general term for conditions affecting the heart or blood vessels. It's usually associated with a build-up of fatty deposits inside the arteries (artherosclerosis) and an increased risk of blood clots. It can also be associated with damage to arteries in organs such as the brain, heart, kidneys and eyes. As used herein, CVD intents subjects with active disease or at high risk of such disease.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

As used herein, “cancer” or “malignancy” or “tumor” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features.

A “solid tumor” is an abnormal mass of tissue that usually does not contain cysts or liquid areas. Solid tumors can be benign or malignant. Different types of solid tumors are named for the type of cells that form them. Examples of solid tumors include, but not limited to, sarcomas, carcinomas, and lymphomas. In some embodiments, a solid tumor comprises bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, skin cancer, gastric cancer, esophageal cancer, colon cancer, glioma, cervical cancer, hepatocellular, thyroid cancer, or stomach cancer.

As used herein, a “metastatic cancer” is a cancer that spreads from where it originated to another part of the body. Non-limiting examples of such include cancers metastasize to the intraperitoneal cavity, e.g., disseminated metastatic cancer, metastatic ovarian cancer, metastatic colon cancer, metastatic liver cancer, metastatic lung cancer. Other non-limiting examples include metastatic lung cancer, metastatic ovarian cancer, metastatic colon cancer, and metastatic breast cancer.

As used herein, a “cancer cell” are cells that have uncontrolled cell division and form solid tumors or enter the blood stream.

As used herein, the term “administer” or “administration” or “administering” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, sub-cutaneous, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

An agent of the present disclosure can be administered for therapy by any suitable route of administration. It will also be appreciated that the optimal route will vary with the condition and age of the recipient, and the disease being treated.

“Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the chimeric PVX described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. A high stringency hybridization refers to a condition in which hybridization of an oligonucleotide to a target sequence comprises no mismatches (or perfect complementarity). Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: prevention of recurrence, reduction in tumor burden, slowing of tumor growth, longer overall survival, longer time to tumor progression, inhibition of metastasis or a reduction in metastasis of the tumor. In one aspect, treatment excludes prophylaxis.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “enhancer”, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “introduce” as applied to methods of producing modified cells such as chimeric antigen receptor cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest (e.g., AAV). In some embodiments, introduction further comprises CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

S100 calcium-binding protein A9 (S100A9; also known as migration inhibitory factor-related protein 14 or MRP14 or calgranulin B) is a protein involved in cellular processes such as cell cycle progression and differentiation and a central mediator of inflammation in cancer and other diseases. It is a calcium-binding protein that regulates inflammation and while there is some level of endogenous S100A9 expression in the squamous epithelium and mucosal tissues, it becomes overexpressed in many different forms of cancer including breast, ovarian, skin, bladder, pancreatic, gastric, esophageal, colon, glioma, cervical, hepatocellular, and thyroid. It is most commonly found in its heterodimer form with S100A8, but can also be found as a homodimer. S100A8/9 complexes are also found in mice and extensive biochemical characterization has demonstrated functional equivalency with its human counterpart. S100A9 expression is heavily linked with tumor aggressiveness and tumorigenesis through the activation of the nuclear factor-κB (NF-κB) and mitogen-activated protein kinase (MAPK) pathways, which are responsible for inflammation-induced cancer development and uncontrolled cell proliferation respectively. It is mainly expressed and secreted by MDSCs, which promotes further accumulation of MDSCs via autocrine pathways into the tumor microenvironment (TME) in an expanding and cyclic fashion. MDSCs suppress the immune response within the TME through reprogramming of the TME into a protumor phenotype, and tumors soon begin establishing S100A9 gradients of myeloid cell migration.

Bacteriophage Qβ (Qbeta or alternatively QB) is a member of the levivirida family. It is a small virus that is about 25 nm thick and is a coliphage with an RNA that is 4217 nucleotides long. As described in biology.kenyon.edu/BMB/jsmol2019/EAIJ/NewVersion81.html #:˜:text=Bacteriophage%20Qβ%20is%20a%20member,%2C%20%26%20Finn%2C%202009).&text-Members %20of% 20the%20leviviridae%20family,et%20at.%2C %202018) last accessed on Aug. 17, 2021, Qβ has 20 faces each composed of six subunits and 12 vertices each composed of 5 subunits. Members of the leviviridae family form icosahedral capsids from 180 coat protein subunits around a 4.2 kb sense-strand RNA genome. Each of these coat proteins (capsomers) has about 132 residues of amino acids. Bacteriophage QB is a positive strand RNA virus. Positive strand RNA viruses have genomes that are functional mRNAs. For instance, QB's genome codes for 4 proteins: A1, A2, CP and QB replicase. QB has other proteins like the B-subunit of a replicase, the maturation protein A2 and a minor protein A1. The penetration of the virus into a host cell is quickly followed by translation to produce RdRps and other viral proteins that are required for the production of more viral RNAs. QB ssRNA adsorb to bacterial sex pili proteins and infect. Like other RNA viruses, QB replicates its genome by utilizing virally encoded RNA polymerase (RdRp). The genome is used as the template for the synthesis of other RNA strands. Upon infection, the B-subunit interacts with host proteins to form a complex. The complex contains RNA-helicases to unwind DNA and NTPases that are useful for polymerization. Once the complex forms, the transcription of the genome, a copy of the genome, and mRNAs begin. Phage MS2 has the same genome as QB.

Bacteriophage Qβ coat protein self-assembles to form an icosahedral capsid with a T=3 symmetry, about 26 nm in diameter, and consisting of 89 capsid proteins dimers (178 capsid proteins). It is also involved in viral genome encapsidation through the interaction between a capsid protein dimer and the multiple packaging signals present in the RNA genome. Binding of the capsid proteins to the viral RNA induces a conformational change required for efficient T=3 shell formation. Additionally, it acts as a translational repressor of viral replicase synthesis late in infection. This latter function is the result of capsid protein interaction with an RNA hairpin which contains the replicase ribosome-binding site. See, for example, Gorzelnik et al., Proc Natl Acad Sci USA. 2016 Oct. 11; 113 (41): 11519-11524; Basnak et al., J Mol Biol. 2010 Feb. 5; 395 (5): 924-36; and Lim et al., J Biol Chem. 1996 Dec. 13; 271 (50): 31839-45.

In some embodiments, a VLP derived from bacteriophage Qβ comprises, or consists essentially of, or yet further consists of, a plurality of coat proteins (CPs). In some embodiments, the coat protein is a wild-type bacteriophage Qβ coat protein. In further embodiments, the coat protein is modified, e.g., comprising, or consisting essentially of, or yet further consisting of, one or more substitutions, insertions, and/or deletions. In some embodiments, a bacteriophage Qβ coat protein comprises, or alternatively consists essentially of, or yet further consists of the sequence as set forth in the UniProtKB ID P03615: MAKLETVTLGNIGKDGKQTLVLNPRGVNPTNGVASLSQAGAVPALEKRVTVSVSQP SRNRKNYKVQVKIQNPTACTANGSCDPSVTRQAYADVTFSFTQYSTDEERAFVRTEL AALLASPLLIDAIDQLNPAY (SEQ ID NO: 5) or an equivalent thereof.

A bacteriophage Qβ hairpin loop refers to a portion of a Qβ RNA where a Qβ coat protein can bind to. In some embodiments, the hairpin loop serves as a packaging signal directing an RNA comprising the hairpin loop to be encapsidated in a capsid comprising, or consisting essentially of, or yet further consisting of a Qβ coat protein.

Applicant utilizes herein a vaccine targeting peptides S100A9 peptide: 101-₁₁₀[PGHHHKPGLG]₁₁₀ (human) (SEQ ID NO: 1) or ₁₀₁[RGHGHSHGKG]₁₁₀ (murine) (SEQ ID NO: 2).

### Virus-Like Particles (VLPs)

VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. VLPs can also be engineered, e.g., comprising, or consisting essentially of, or yet further consisting of, one or more viral proteins that comprise, or consist essentially of, or yet further consist of, a modification. Methods for producing VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Viral. 68:4503-4505; Vincente, J Invertebr Pathol., 2011; Schneider Ohrum and Ross, Curr. Top. Microbial. Immunol., 354:53073, 2012).

In some embodiments, the virus or VLP is derived from Cowpea mosaic virus (CPMV). CPMV is a non-enveloped plant virus that belongs to the Comovirus genus. CPMV strains include, but are not limited to, SB (Agrawal, H. O. (1964). Meded. Landb. Hoogesch. Wagen. 64:1) and Vu (Agrawal, H. O. (1964). Meded. Landb. Hoogesch. Wagen. 64:1).

In some instances, the virus or VLP from CPMV comprises, or consists essentially of, or yet further consists of, a plurality of capsid proteins. In some instances, CPMV produces a large capsid protein and a small capsid protein precursor (which generates a mature small capsid protein). In some cases, CPMV capsid is formed from a plurality of large capsid proteins and mature small capsid proteins. In some cases, the large capsid protein is a wild-type large capsid protein, optionally expressed by SB or Vu strain. In other instances, the large capsid protein is a modified large capsid protein, e.g., comprising, or consisting essentially of, or yet further consisting of, one or more substitutions, insertions, and/or deletions. In some cases, the large capsid protein comprises, or consists essentially of, or yet further consists of, the sequence as set forth in the UniProtKB ID P03599 (residues 460-833):

MEQNLFALSLDDTSSVRGSLLDTKFAQTRVLLSKAMAGGDVLLDEYLYDVV NGQDFRATVAFLRTHVITGKIKVTATTNISDNSGCCLMLAINSGVRGKYSTDVYTICS QDSMTWNPGCKKNFSFTFNPNPCGDSWSAEMISRSRVRMTVICVSGWTLSPTTDVIA KLDWSIVNEKCEPTIYHLADCQNWLPLNRWMGKLTFPQGVTSEVRRMPLSIGGGAG ATQAFLANMPNSWISMWRYFRGELHFEVTKMSSPYIKATVTFLIAFGNLSDAFGFYE SFPHRIVQFAEVEEKCTLVFSQQEFVTAWSTQVNPRTTLEADGCPYLYAIIHDSTTGTI SGDFNLGVKLVGIKDFCGIGSNPGIDGSRLLGAIAQ (SEQ ID NO: 3), or an equivalent thereof.

In some cases, the mature small capsid protein is a wild-type mature small capsid protein, optionally expressed by SB or Vu strain. In other instances, the mature small capsid protein is a modified mature small capsid protein, e.g., comprising, or consisting essentially of, or yet further consisting of, one or more substitutions, insertions, and/or deletions. In some cases, the mature small capsid protein comprises, or consists essentially of, or yet further consists of, the sequence as set forth in the UniProtKB ID P03599 (residues 834-1022):

GPVCAEASDVYSPCMIASTPPAPFSDVTAVTFDLINGKITPVGDDNWNTHIYN PPIMNVLRTAAWKSGTIHVQLNVRGAGVKRADWDGQVFVYLRQSMNPESYDARTF VISQPGSAMLNFSFDIIGPNSGFEFAESPWANQTTWYLECVATNPRQIQQFEVNMRFD PNFRVAGNILMPPFPLSTETPPL (SEQ ID NO: 4), or an equivalent thereof.

In some aspects, VLP is or is derived from Bacteriophage Qβ (Qbeta or alternatively Qbeta bacteriophage) which is a member of the levivirida family.

In some embodiments, a VLP derived from bacteriophage Qβ comprises, or consists essentially of, or yet further consists of, a plurality of coat proteins. In some embodiments, the coat protein is a wild-type bacteriophage Qβ coat protein. In further embodiments, the coat protein is modified, e.g., comprising, or consisting essentially of, or yet further consisting of, one or more substitutions, insertions, and/or deletions. In some embodiments, a bacteriophage Qβ coat protein comprises, or alternatively consists essentially of, or yet further consists of the sequence as set forth in the UniProtKB ID P03615: MAKLETVTLGNIGKDGKQTLVLNPRGVNPTNGVASLSQAGAVPALEKRVTVSVSQP SRNRKNYKVQVKIQNPTACTANGSCDPSVTRQAYADVTFSFTQYSTDEERAFVRTEL AALLASPLLIDAIDQLNPAY (SEQ ID NO: 5) or an equivalent thereof.

As used herein, the term “an equivalent thereof” in reference to a polynucleotide or a protein (e.g., a capsid or coat protein) include a polynucleotide or a protein that comprises, or consists essentially of, or yet further consists of, at least 80%, 85%, 90%, 95%, 96%, 97%, 98%, or 99% sequence identify to the respective polynucleotide or protein of which it is compared to, while still retaining a functional activity. In the instances with reference to a capsid or coat protein, a functional activity refers to the formation of a virus or VLP.

As used herein, the term “modification” include, for example, substitutions, additions, insertions and deletions to the amino acid sequences, which can be referred to as “variants.” Exemplary sequence substitutions, additions, and insertions include a full length or a portion of a sequence with one or more amino acids substituted (or mutated), added, or inserted, for example of a capsid derived from the plant virus. In some instances, a capsid described herein includes, e.g., a modified capsid comprising, or consisting essentially of, or yet further consisting of, at least 70%, 80%, 85%, 90%, 95%, 96%, 97%, 98%, 99%, or 100% sequence identity to its respective wild-type version.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. A polynucleotide or polynucleotide region (or a polypeptide or polypeptide region) having a certain percentage (for example, 60%, 65%, 70%, 75%, 80%, 85%, 90%, 95%, 98% or 99%) of “sequence identity” to a reference sequence means that, when aligned, that percentage of bases (or amino acids) at each position in the test sequence are identical to the base (or amino acid) at the same position in the reference sequence. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/blast/Blast.cgi.

Modified capsid polypeptides include, for example, non-conservative and conservative substitutions of the capsid amino acid sequences.

As used herein, the term “conservative substitution” denotes the replacement of an amino acid residue by another, chemically or biologically similar residue. Biologically similar means that the substitution does not destroy a biological activity or function, e.g., assembly of a viral capsid. \Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such proteins that include amino acid substitutions can be encoded by a nucleic acid. Consequently, nucleic acid sequences encoding proteins that include amino acid substitutions are also provided.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide.

In some instances, a virus or VLP described herein further comprises, or consists essentially of, or yet further consists of, a label or a tag, e.g., such as a detectable label. A detectable label can be attached to, e.g., to the surface of a virus or VLP.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As, ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰⁹Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Ffe(III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) to the virus or VLP or nanoparticle. In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

### The VLP can be Detectably Labeled.

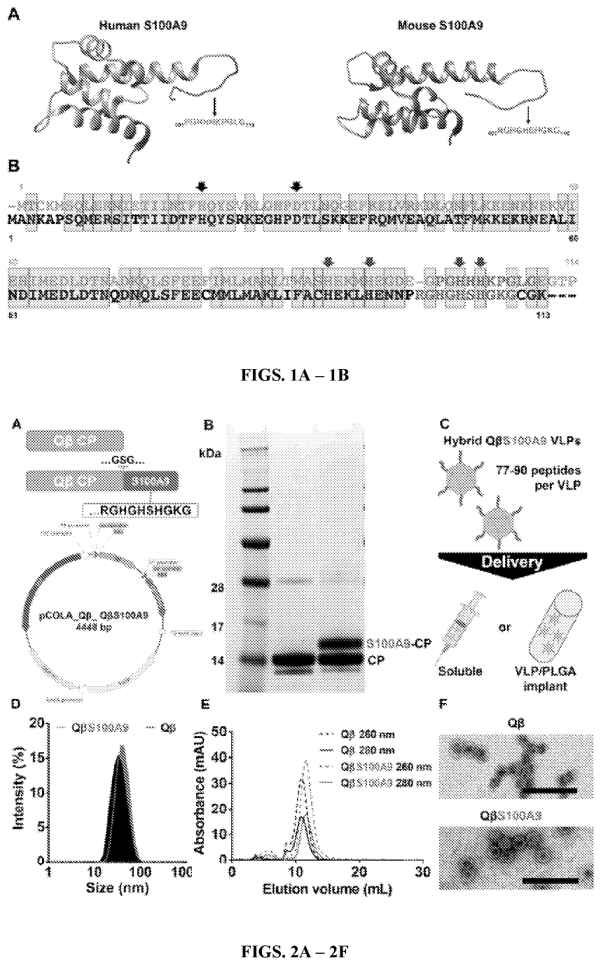

Also provided herein is the virus, VLP, or nanoparticle as described herein comprises, or consists essentially of, or yet further consists of the peptide that recognizes and binds the 101- to 110 epitope of S100A9 (FIG. 1) and an additional therapeutic agent. In some cases, the additional therapeutic agent disclosed herein comprises, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the virus, VLP, or nanoparticle with or without the additional therapeutic agent comprises, or consists essentially of, or yet further consists of, or is used as a first-line therapy. As used herein, “first-line therapy” comprises, or consists essentially of, or yet further consists of, a primary treatment for a subject with a cancer. In some instances, the cancer is a primary cancer. In other instances, the cancer is a metastatic or recurrent cancer. In some cases, the first-line therapy comprises, or consists essentially of, or yet further consists of, chemotherapy. In other cases, the first-line treatment comprises, or consists essentially of, or yet further consists of, radiation therapy. A skilled artisan would readily understand that different first-line treatments may be applicable to different type of cancers.

In some cases, the additional therapeutic agent comprises, or consists essentially of, or yet further consists of, or is used as a second-line therapy, a third-line therapy, a fourth-line therapy, or a fifth-line therapy. As used herein, a second-line therapy encompasses treatments that are utilized after the primary or first-line treatment stops. They can also be used as third-line, fourth-line or fifth line therapy. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprises, or consists essentially of, or yet further consists of, a salvage therapy.

In some cases, the additional therapeutic agent comprises, or consists essentially of, or yet further consists of, a palliative therapy.

In connection with the treatment of CVD and associated disorders, the disclosed compositions can be combined with other therapies that comprise, or consist essentially of, or yet further consist of, statins to reduce plasma cholesterol levels, angioplasty, lifestyle changes, beta blockers, nitrates, angiotensin-converting enzyme inhibitors, angiotensin-2 receptor blockers, calcium channel blockers and diuretics.

In connection with cancer care, the treatment can comprise an additional therapeutic agent that comprises, or consists essentially of, or yet further consists of, an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULA®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprises, or consists essentially of, or yet further consists of, an immune checkpoint inhibitor. Exemplary checkpoint inhibitors include:
PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736;
PD-L2 inhibitors such as GlaxoSmithKline's AMP-224 (Amplimmune), and rHIgM12B7;
PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd;
CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam;
LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12; B7-H3 inhibitors such as MGA271;
KIR inhibitors such as Lirilumab (IPH2101);
CD137 inhibitors such as urelumab (BMS-663513, Bristol-Myers Squibb), PF-05082566 (anti-4-1BB, PF-2566, Pfizer), or XmAb-5592 (Xencor);
PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some cases, the additional therapeutic agent comprises, or consists essentially of, or yet further consists of, pembrolizumab, nivolumab, tremelimumab, or ipilimumab.

In some cases, the additional therapeutic agent comprises, or consists essentially of, or yet further consists of, an antibody such as alemtuzumab, trastuzumab, ibritumomab tiuxetan, brentuximab vedotin, ado-trastuzumab emtansine, or blinatumomab.

In some cases, the additional therapeutic agent comprises, or consists essentially of, or yet further consists of, a cytokine. Exemplary cytokines include, but are not limited to, IL-Iβ, IL-6, IL-7, IL-10, IL-12, IL-15, IL-21, or TNFα.

In some embodiments, the additional therapeutic agent comprises, or consists essentially of, or yet further consists of, a receptor agonist. In some instances, the receptor agonist comprises, or consists essentially of, or yet further consists of, a Toll-like receptor (TLR) ligand. In some cases, the TLR ligand comprises, or consists essentially of, or yet further consists of, TLR1, TLR2, TLR3, TLR4, TLR5, TLR6, TLR7, TLR8, or TLR9. In some cases, the TLR ligand comprises, or consists essentially of, or yet further consists of, a synthetic ligand such as, for example, Pam3Cys, CFA, MALP2, Pam2Cys, FSL-1, Hib-OMPC, Poly I:C, poly A:U, AGP, MPL A, RC-529, MDF2p, CFA, or Flagellin.

In some cases, the additional therapeutic agent comprises, or consists essentially of, or yet further consists of, an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprises, or consists essentially of, or yet further consists of, use of allogeneic T lymphocytes with, e.g., anti-tumor activity, expansion of the T lymphocytes in vitro, and subsequent infusion of the expanded allogeneic T lymphocytes into a subject in need thereof.

In some instances, the additional therapeutic agent is, or can be used as a vaccine, optionally, an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some instances, the virus or VLP or nanoparticle formulation described herein is administered in combination with a radiation therapy.

### MODES FOR CARRYING OUT THE DISCLOSURE

### VLP Nanoparticles

In some embodiments, the VLP nanoparticle comprises, or alternatively consists essentially of, or yet further consists of a virus or VLP such as CPMV or a Qβ bacteriophage and the S100A9 peptide: 101 [PGHHHKPGLG] 110 (human) (SEQ ID NO: 1) or 101 [RGHGHSHGKG] 110 (murine) (SEQ ID NO: 2). In some aspects, the S100A9 targeting peptide further comprises a linker, that optionally contains a c-terminal cysteine, e.g., GGGSC or the GSG linker. In some embodiments, the virus or VLP has an exposed lysine side chain. The peptide can further comprise a linker peptide.

The nanoparticle, virus or VLP and/or targeting peptide can be detectably labeled for diagnostic or research purposes. Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰⁹Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Ffe(III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

Also provided herein is the virus or VLP as described herein further comprising, or consisting essentially of, or yet further consisting of the peptide that recognizes and binds S100A9 peptide: ₁₀₁[PGHHHKPGLG]₁₁₀ (human) (SEQ ID NO: 1) or ₁₀₁[RGHGHSHGKG]₁₁₀ (murine) (SEQ ID NO: 2) and an additional therapeutic agent. In some cases, the additional therapeutic agent disclosed herein comprises, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone. The additional therapeutic can be conjugated to the virus or VLP using methods known in the art and as described herein.

In connection with cancer care, the treatment can comprise an additional therapeutic agent that comprises, or consists essentially of, or yet further consists of, an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULA®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprises, or consists essentially of, or yet further consists of, an immune checkpoint inhibitor. Exemplary checkpoint inhibitors include: PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736; PD-L2 inhibitors such as GlaxoSmithKline's AMP-224 (Amplimmune), and rHIgM12B7; PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd; CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam; LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12; B7-H3 inhibitors such as MGA271; KIR inhibitors such as Lirilumab (IPH2101); CD137 inhibitors such as urelumab (BMS-663513, Bristol-Myers Squibb), PF-05082566 (anti-4-1BB, PF-2566, Pfizer), or XmAb-5592 (Xencor); PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some cases, the additional therapeutic agent comprises, or consists essentially of, or yet further consists of, pembrolizumab, nivolumab, tremelimumab, or ipilimumab.

In some cases, the additional therapeutic agent comprises, or consists essentially of, or yet further consists of, an antibody such as alemtuzumab, trastuzumab, ibritumomab tiuxetan, brentuximab vedotin, ado-trastuzumab emtansine, or blinatumomab.

In some cases, the additional therapeutic agent comprises, or consists essentially of, or yet further consists of, a cytokine. Exemplary cytokines include, but are not limited to, IL-Iβ, IL-6, IL-7, IL-10, IL-12, IL-15, IL-21, or TNFα.

In some embodiments, the additional therapeutic agent comprises, or consists essentially of, or yet further consists of, a receptor agonist. In some instances, the receptor agonist comprises, or consists essentially of, or yet further consists of, a Toll-like receptor (TLR) ligand. In some cases, the TLR ligand comprises, or consists essentially of, or yet further consists of, TLR1, TLR2, TLR3, TLR4, TLR5, TLR6, TLR7, TLR8, or TLR9. In some cases, the TLR ligand comprises, or consists essentially of, or yet further consists of, a synthetic ligand such as, for example, Pam3Cys, CFA, MALP2, Pam2Cys, FSL-1, Hib-OMPC, Poly I: C, poly A: U, AGP, MPL A, RC-529, MDF2p, CFA, or Flagellin.

In some instances, the additional therapeutic agent is, or can be used as a vaccine, optionally, an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some embodiments the peptide can be chemically conjugated or genetically fused to the CMPV or Qβ CP. Any bioconjugation or chemical conjugation method would be applicable for the conjugation. Non-limiting examples of chemical conjugation include conjugating a thiol-terminated peptide through a maleimide-PEG-NHS linker targeting lysine groups on the virus or VLP, e.g., CMPV or Qβ CP. In some embodiments, a lysine side chain is conjugated to a N-hydroxysuccinimide (NHS) ester and the maleimide of a maleimide-polyethylene glycols is conjugated with the c-terminal cysteine of the targeting peptide. Azide/alkyne modified peptides and virus or VLP (CMPV or Qβ CP) and click chemistry can also be used for chemical conjugation. For bioconjugation such as genetic fusion, the peptide is added as N-terminal fusion in a CMPV or Qβ CP bacteriophage containing the entire VLP. Alternatively, the VLP and S100A9 peptide are recombinantly produced as described herein.

This disclosure also provides alternative method for conjugation of the peptide epitope to the VLP. The peptide epitope can be chemically conjugated or genetically fused to the VLP. Non-limiting examples of chemical conjugation include conjugating a thiol-terminated peptide through a maleimide-PEG-NHS linker targeting lysine groups on the VLP. Azide/alkyne modified peptides and VLP and click chemistry can also be used for chemical conjugation. Any bioconjugation method would be applicable. For genetic fusion, the peptide is added as N-terminal fusion in a VLP encoding plasmid, VLP with the peptide epitope can be produced simply by inoculating plants with the plasmid or by agroinfiltration method.

In some embodiments, the diameter of the nanoparticle disclosed herein, is from about 10 nm to 50 nm. In some embodiments, the diameter may range from about 10 nm, about 15 nm, about 20 nm, about 25 nm, about 30 nm, about 35 nm, about 40 nm, about 45 nm, to about 50 nm.

In some embodiments, a polynucleotide encodes a nanoparticle and S100A9 peptide as disclosed herein that can include regulatory elements, promoters, enhancer and the like, for expression and/or replication. In some embodiments, a vector as disclosed herein, comprises, or alternatively consists essentially of, or yet further consists of a polynucleotide encoding the VLP and S100A9 peptide as disclosed herein. In one aspect, the vector is a plasmid.

Also provided is a host cell that comprises, or alternatively consists essentially of, or yet further consists of a virus, VLP, nanoparticle, vector or polynucleotide as disclosed herein. In one aspect, the vector is a plasmid. In one aspect, the host cell is a prokaryotic cell. In another aspect, the host cell is a eukaryotic cell. In one particular aspect, the host cell is a plant cell or a bacterium.

### Compositions

In another aspect, provided herein is a composition comprising, consisting essentially of, or consisting of the combination of formulations comprising a virus, VLP, nanoparticle, polynucleotide, or host cell as provided herein, and at least one carrier, such as a pharmaceutically acceptable carrier or excipient. In one aspect, the composition further comprises a preservative or stabilizer.

In one embodiment, this technology relates to a composition comprising a combination of VLP or formulations as described herein and a carrier.

In another embodiment, this technology relates to a pharmaceutical composition comprising a combination of virus, VLP, nanoparticles or formulations as described herein and a pharmaceutically acceptable carrier.

In another embodiment, this technology relates to a pharmaceutical composition comprising an effective amount or a therapeutically effective amount of a combination of virus, VLP, nanoparticle formulations as described herein and a pharmaceutically acceptable carrier.

Compositions, including pharmaceutical compositions comprising, consisting essentially of, or consisting of the nanoparticle formulation alone or in combination of other therapeutic agents can be manufactured by means of conventional mixing, dissolving, granulating, dragee-making levigating, emulsifying, encapsulating, entrapping, or lyophilization processes. These can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical formulations described herein are administered to a subject by multiple administration routes, including but not limited to, parenteral, oral, buccal, rectal, sublingual, or transdermal administration routes. In some cases, parenteral administration comprises, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL®, dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL® e, or sodium starch glycolate such as PROMOGEL® or EXPLOTAB®, a cellulose such as a wood product, methylcrystalline cellulose, e.g., AVICEL®, AVICEL® PH101, AVICEL® PH102, AVICEL® PH105, ELCEMAR P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOL®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorb ate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The pharmaceutical compositions for the administration of the combinations of compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the combination of compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

In some embodiments, one or more compositions disclosed herein are contained in a kit. Accordingly, in some embodiments, provided herein is a kit comprising, consisting essentially of, or consisting of one or more compositions disclosed herein and instructions for their use.

### Dosage and Dosage Formulations

In some embodiments, the compositions are administered to a subject suffering from a condition as disclosed herein, such as a human, either alone or as part of a pharmaceutically acceptable formulation, once a week, once a day, twice a day, three times a day, or four times a day, or even more frequently.

Administration of the virus, VLP, VLPs or nanoparticle formulation alone or in combination with the additional therapeutic agent and compositions containing same can be effected by any method that enables delivery to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values can vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration are well-known in the relevant art and would be understood to be encompassed by the skilled artisan once provided the teachings disclosed herein.

### Diagnostic Methods

In some embodiments, one or more of the methods described herein further comprises, or consists essentially of, or yet further consists of, a diagnostic step. In some instances, a sample is first obtained from a subject suspected of having a disease or condition described above. Exemplary samples include, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

Various methods known in the art can be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

The compositions of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

### Therapeutic Methods

Further disclosed herein are methods for inducing an immune response in a subject consisting essentially of, or yet further consisting of the virus, VLP or nanoparticles, polynucleotides, vectors and/or host cells as disclosed herein.

Also provided are methods for targeting S100A9 peptide: ₁₀₁[PGHHHKPGLG]₁₁₀ (human) (SEQ ID NO: 1) or ₁₀₁[RGHGHSHGKG]₁₁₀ (murine) (SEQ ID NO: 2), inducing an immune reaction, treating CVD, treating cancer, treating metastatic cancer and associated disorders, the methods comprising, or consisting essentially of, or consisting of contacting tissue in need of such therapy or expressing S100A9 peptide: ₁₀₁[PGHHHKPGLG]₁₁₀ (human) (SEQ ID NO: 1) or ₁₀₁[RGHGHSHGKG]₁₁₀ (murine) (SEQ ID NO: 2), with the virus, VLP, nanoparticle, polynucleotide, vector, the composition and/or the host cell of this disclosure. The contacting can be in vitro or in vivo.

Further disclosed herein are methods for inducing an immune response, treating CVD, treating cancer, treating metastatic cancer and associated disorders, the methods comprising, or consisting essentially of, or consisting of contacting tissue in need of such therapy or expressing S100A9 101, in a subject in need thereof, comprising, or alternatively consisting essentially of, or yet further consisting of administering to the subject the virus, VLP, nanoparticles, polynucleotides, vectors and/or host cells as disclosed herein.

Further disclosed herein are methods for altering an immune cell profile in lungs of a subject comprising, or alternatively consisting essentially of, or yet further consisting of virus, VLP, nanoparticles, polynucleotides, vectors and/or host cells as disclosed herein.

In some embodiments, a subject is a mammal. In some embodiments, a subject is a human. In some embodiments, a subject has a condition. In some embodiments, a subject has CVD or separately cancer. In some embodiments, a cancer is selected from melanoma, breast cancer, prostate cancer, lung cancer, ovarian cancer, skin cancer, bladder cancer, pancreatic cancer, gastric cancer, esophageal cancer, colon cancer, glioma, cervical cancer, hepatocellular cancer, or thyroid cancer. In some embodiments, the cancer is primary or metastatic cancer. In some embodiments, the cancer is metastatic or primary lung cancer or breast cancer. In some embodiments, the cancer metastatic melanoma or metastatic triple negative breast cancer. In some embodiments, the cancer is a primary or metastatic cancer in lung. In some embodiments, the cancer expresses S100A9.

In some embodiments, administering is selected from intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, or inhalation. In some embodiments, administering is intravenous.

The methods and compositions disclosed herein may further comprise or alternatively consist essentially of, or yet further consists of administering to the subject an anti-tumor therapy other than the virus, VLP, nanoparticle disclosed herein. In some embodiments, anti-tumor therapy may include different cancer therapy or tumor resection. The additional therapeutic can be combined in the same composition or separately administered.

In some embodiments, the VLP, nanoparticle and/or composition are provided to prevent the symptoms of cancer from occurring in a subject that is predisposed or does not yet display symptoms of the cancer.

In some embodiments, the virus, VLP, polynucleotide, nanoparticle, vector, or composition disclosed herein may be delivered or administered into a cavity formed by the resection of tumor tissue (i.e. intracavity delivery) or directly into a tumor prior to resection (i.e. intratumoral delivery). In some embodiments. In some embodiments, the administering is intravenous.

In some embodiments, any of the virus, VLP, polynucleotides, nanoparticles, vectors, or compositions disclosed herein, are administered to the subject at least 1, 2, 3, 4, 5, 6, 7, 8, 9, or 10 times a day. In some embodiments, any of polynucleotides, nanoparticles, vectors, or compositions disclosed herein are administered to the subject at least 1, 2, 3, 4, 5, 6, 7, 8, 9, 10, 11, 12, 13, 14, 15, 16, 17, 18, 19, or 20 times a week. In some embodiments, any of the polynucleotides, nanoparticles, vectors, or compositions disclosed herein are administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, any of the virus, VLP, polynucleotides, nanoparticles, vectors, or compositions disclosed herein are administered to the subject at least every 1, 2, 3, 4, 5, 6, 7, 8, 9, or 10 days. In some embodiments, any of the virus, VLP, polynucleotides, nanoparticles, vectors, or compositions disclosed herein are administered to the subject at least every 1, 2, 3, 4, 5, 6, 7, 8, 9, 10, 11, 12, 13, or 14 weeks. In some embodiments, any of the virus, VLP, polynucleotides, nanoparticles, vectors, or compositions disclosed herein are administered to the subject for a period of at least 1, 2, 3, 4, 5, 6, 7, 8, 9, or 10 days. In some embodiments, any of the virus, VLP, polynucleotides, nanoparticles, vectors, or compositions disclosed herein are administered to the subject for a period of at least 1, 2, 3, 4, 5, 6, 7, 8, 9, 10, 11, 12, 13, 14, 15, 16, 17, 18, 19, or 20 weeks. In some embodiments, any of the virus, VLP, polynucleotides, nanoparticles, vectors, or compositions disclosed herein are administered to the subject for a period of at least 1, 2, 3, 4, 5, 6, 7, 8, 9, 10, 12, 13, 14, 15, 16, 17, 18, 19, or 20 months.

In some embodiments, the method and compositions provided herein, comprising, or alternatively consisting essentially of, or yet further consisting inhibiting metastatic potential of the cancer, reduction in tumor size, a reduction in tumor burden, longer progression free survival, or longer overall survival of the subject.

In one aspect, the methods or compositions further comprise administration of an additional therapeutic agent. In some cases, the additional therapeutic agent disclosed herein comprises, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

When the disease to be treated is cancer, the virus, VLP, nanoparticle with or without the additional therapeutic agent comprises, or consists essentially of, or yet further consists of, or is used as a first-line therapy. As used herein, “first-line therapy” comprises, or consists essentially of, or yet further consists of, a primary treatment for a subject with a cancer. In some instances, the cancer is a primary cancer. In other instances, the cancer is a metastatic or recurrent cancer. In some cases, the first-line therapy comprises, or consists essentially of, or yet further consists of, chemotherapy. In other cases, the first-line treatment comprises, or consists essentially of, or yet further consists of, radiation therapy. A skilled artisan would readily understand that different first-line treatments may be applicable to different type of cancers.

In some cases, the additional therapeutic agent comprises, or consists essentially of, or yet further consists of, or is used as a second-line therapy, a third-line therapy, a fourth-line therapy, or a fifth-line therapy. As used herein, a second-line therapy encompasses treatments that are utilized after the primary or first-line treatment stops. They can also be used as third-line, fourth-line or fifth line therapy. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprises, or consists essentially of, or yet further consists of, a salvage therapy.

In some cases, the additional therapeutic agent comprises, or consists essentially of, or yet further consists of, a palliative therapy.

In connection with cancer care, the treatment can comprise an additional therapeutic agent that comprises, or consists essentially of, or yet further consists of, an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULAR, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprises, or consists essentially of, or yet further consists of, an immune checkpoint inhibitor. Exemplary checkpoint inhibitors include: PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736; PD-L2 inhibitors such as GlaxoSmithKline's AMP-224 (Amplimmune), and rHIgM12B7; PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd; CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam; LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12; B7-H3 inhibitors such as MGA271; KIR inhibitors such as Lirilumab (IPH2101); CD137 inhibitors such as urelumab (BMS-663513, Bristol-Myers Squibb), PF-05082566 (anti-4-1BB, PF-2566, Pfizer), or XmAb-5592 (Xencor); PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. In some cases, the additional therapeutic agent comprises, or consists essentially of, or yet further consists of, pembrolizumab, nivolumab, tremelimumab, or ipilimumab.

In some cases, the additional therapeutic agent comprises, or consists essentially of, or yet further consists of, an antibody such as alemtuzumab, trastuzumab, ibritumomab tiuxetan, brentuximab vedotin, ado-trastuzumab emtansine, or blinatumomab.

In some cases, the additional therapeutic agent comprises, or consists essentially of, or yet further consists of, a cytokine. Exemplary cytokines include, but are not limited to, IL-Iβ, IL-6, IL-7, IL-10, IL-12, IL-15, IL-21, or TNFα.

In some embodiments, the additional therapeutic agent comprises, or consists essentially of, or yet further consists of, a receptor agonist. In some instances, the receptor agonist comprises, or consists essentially of, or yet further consists of, a Toll-like receptor (TLR) ligand. In some cases, the TLR ligand comprises, or consists essentially of, or yet further consists of, TLR1, TLR2, TLR3, TLR4, TLR5, TLR6, TLR7, TLR8, or TLR9. In some cases, the TLR ligand comprises, or consists essentially of, or yet further consists of, a synthetic ligand such as, for example, Pam3Cys, CFA, MALP2, Pam2Cys, FSL-1, Hib-OMPC, Poly I: C, poly A: U, AGP, MPL A, RC-529, MDF2p, CFA, or Flagellin.

In some cases, the additional therapeutic agent comprises, or consists essentially of, or yet further consists of, an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprises, or consists essentially of, or yet further consists of, use of allogeneic T lymphocytes with, e.g., anti-tumor activity, expansion of the T lymphocytes in vitro, and subsequent infusion of the expanded allogeneic T lymphocytes into a subject in need thereof.

In some instances, the additional therapeutic agent is, or can be used as a vaccine, optionally, an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some instances, the VLP formulation described herein is administered in combination with a radiation therapy.

### Kits

In one particular aspect, the present disclosure provides kits for performing the methods of this disclosure as well as instructions for carrying out the methods of the present disclosure. The kit comprises, or alternatively consists essentially of, or yet further consists of one or more of virus, VLP, nanoparticle, polynucleotide, vector and/or host cell of this disclosure and instructions for use. In a further aspect, the instruction for use provide directions to conduct any of the methods disclosed herein.

The kits are useful for detecting the presence of cancer such as lung cancer in a biological sample e.g., any bodily fluid including, but not limited to, e.g., sputum, serum, plasma, lymph, cystic fluid, urine, stool, cerebrospinal fluid, acitic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kit components, (e.g., reagents) can be packaged in a suitable container. The kit can also comprise, or alternatively consist essentially of, or yet further consist of, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

As is apparent to those of skill in the art, the aforementioned methods and compositions can be combined with other therapeutic composition and agents for the treatment or the disclosed diseases or conditions.

### Experiment No. 1:

Applicant provides a vaccine for the treatment of metastatic cancer and cardiovascular disease. For atherosclerosis treatment the disclosed embodiment of the Qβ-S100A9 vaccine maintains the plasma levels of calprotectin (S100A8/S100A9 heterodimer) at physiological levels vs control group (highly increased) after atherosclerosis induction with high-fat diet, and as mechanism of action (MOA) Applicant observed that this reduction in plasma levels of calprotectin was correlated with the reduction in plasma level of different pro-inflammatory cytokines and chemokines as well (IL-1β, IL-6, and MCP-1). For the treatment of cancer metastasis, Applicant demonstrated that the Qβ-S100A9 and CPMV-S100A9 vaccines against S100A9 significantly reduced metastatic tumor burden within the lungs of mice through the reduction levels of S100A9.

Applicant chose S100A9 epitope ₁₀₁RGHGHSHGKG₁₁₀ (SEQ ID NO: 2) based on its score (0.5 threshold) as a B-cell epitope as determined using the BepiPred-2.0 Sequential B-Cell Epitope Predictor. Epitope ₁₀₄GHSHGKGCGK₁₁₃ (SEQ ID NO: 6), which includes amino acids 111-113 (CGK), was determined that those amino acid residues did not pass the score threshold and they were therefore excluded.

Applicant's Qβ-S100A9 vaccine is thermo-stable and can prepared as a slow-release poly(lactic-co-glycolic acid) (PLGA) implant using hot melt-extrusion. This process does not compromise the structural properties or immunogenicity of the vaccine and can be store at room temperature for long term.

For CVD applications, the efficacy and safety of the vaccine was tested in a traditional prime-boost-boost schedule vs the single-dose slow-release injectable implant in healthy mice and in the ApoE−/− model of atherosclerosis by measuring antibody titers and immune responses, plasma levels of calprotectin, IL-1β, IL-6, and MCP-1, and the severity of aortic lesions in the aortic arch and thoracic aorta.

Applicant developed a CVD vaccine targeting the S100A9 protein in order to reduce serum levels of calprotectin. The success of any vaccine requires the use of a suitable antigen combined with a strong adjuvant and an effective delivery strategy. Applicant therefore used virus-like particles (VLPs) from bacteriophage Qβ to display a B-cell epitope from the mouse S100A9 protein (FIG. 1). Qβ VLPs can be produced in large quantities by the expression of a recombinant Qβ 14-kDa capsid protein in Escherichia coli (in vivo assembly), and scalable production according to current good manufacturing practices (cGMP) has been demonstrated for several Qβ-based VLP vaccine candidates in clinical trials³¹. To prolong the delivery of the vaccine, Applicant utilized a slow-release poly(lactic-co-glycolic acid) (PLGA) implant, which was mixed with the VLP vaccines using hot melt-extrusion. This process does not compromise the structural properties or immunogenicity of the vaccine^{28, 29, 32, 33, 34}. Efficacy and safety of the vaccine was tested in a traditional prime-boost-boost schedule vs the single-dose slow-release injectable implant in healthy mice and in the ApoE−/− model of atherosclerosis by measuring antibody titers and immune responses, plasma levels of calprotectin, IL-1β, IL-6, and MCP-1, and the severity of aortic lesions in the aortic arch and thoracic aorta.

### Results and Discussion

### Characterization of QβS100A9 Particles

Applicant chose S100A9 epitope ₁₀₁RGHGHSHGKG₁₁₀ (SEQ ID NO: 2) (FIG. 1) based on its score (0.5 threshold) as a B-cell epitope as determined using the BepiPred-2.0 Sequential B-Cell Epitope Predictor³⁵. The epitope ₁₀₄GHSHGKGCGK₁₁₃ (SEQ ID NO: 6), which includes amino acids 111-113 (CGK) had been tested, but Applicant found that those residues did not pass the score threshold and were therefore excluded. The epitope ₁₀₁RGHGHSHGKG₁₁₀ (SEQ ID NO: 2) was fused to the C-terminus of the Qβ coat protein (CP) with an intervening GSG linker, and was expressed in a vector also containing the unmodified Qβ CP gene to allow the assembly of hybrid particles (FIG. 2A). The QβS100A9 vaccine candidate was expressed in E. coli and purified by sucrose gradient ultracentrifugation, with yields of ˜360 mg unmodified Qβ and ˜195 mg QβS100A9 per liter of culture. The lower yield of hybrid particles has been observed before, and probably reflects the negative impact of the additional peptide on VLP assembly²⁷.

Sodium dodecyl sulfate-polyacrylamide gel electrophoresis (SDS-PAGE) analysis (FIG. 2B) confirmed that only wild-type CP (˜14 kDa) was present in the unmodified Qβ VLPs, whereas the hybrid particles contained both the wild-type CP and the larger QβS100A9 CP (˜15.2 kDa). Densitometric comparison revealed that the hybrid particles were composed of 42-50% QβS100A9 CPs, indicating that 77-90 peptide epitopes are displayed per hybrid VLP (180 CP subunits in total) (FIG. 2C). Dynamic light scattering (DLS) revealed the particles were monodisperse with a hydrodynamic diameter (Z-average) of ˜32 nm for unmodified Qβ VLPs and ˜39 nm for QβS100A9 VLPs (FIG. 2D). Fast protein liquid chromatography (FPLC) revealed slightly overlapping major single peaks for unmodified Qβ and QβS100A9, with a similar elution volume of 10-15 mL indicating that both types of VLP were intact and did not form significant or apparent aggregates (FIG. 2E). The unmodified Qβ VLPs had a higher polydispersity index (PDI) of 0.11 compared to 0.01 for the QβS100A9 VLPs, indicating that the QβS100A9 particles were larger but more uniformly distributed, as reported for other Qβ-based vaccines²⁷. Transmission electron microscopy (TEM) of negatively stained particles indicated that the Qβ and QβS100A9 VLPs were intact (FIG. 2F). These results confirm that the QβS100A9 VLPs are stable and the peptide does not affect particle integrity.

### Immunogenicity of Soluble QβS100A9 VLPs and Slow-Release Implants

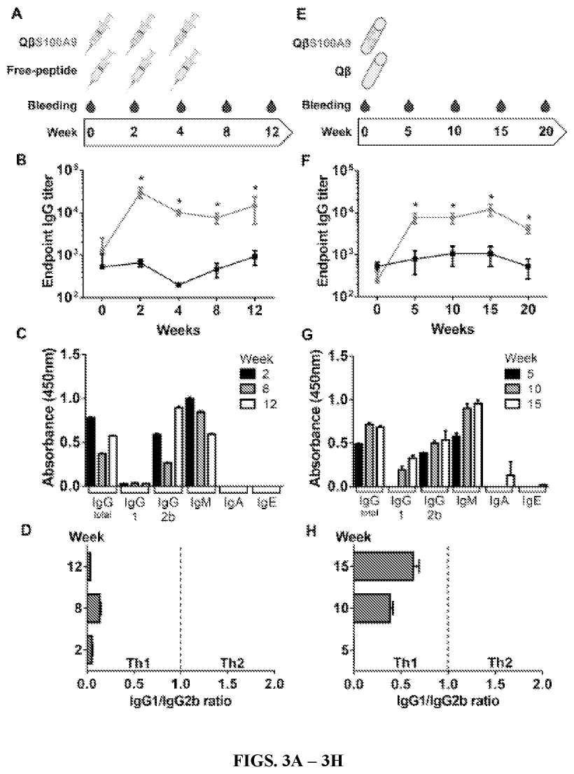

Applicant developed protocols for hot-melt extrusion yielding degradable PLGA: Qβ implants with a ˜1 month release timeframe that achieved the same immunogenicity as a multi-dose regimen of soluble vaccines^{27, 28, 29}. To confirm the immunogenicity of the VLPs before formulation, Applicant compared the soluble QβS100A9 VLPs to the free peptide epitope in a traditional subcutaneous (s.c.) vaccination schedule consisting of a prime plus two boosts, 2 weeks apart. Each injection consisted of 100 μg QβS100A9 VLPs or 5 μg of the free peptide, resulting in a molar equivalent dosage of the epitope in each case (FIG. 3A). As expected, the QβS100A9 VLPs elicited significantly higher specific IgG titers than the free peptide over a period of 12 weeks (FIG. 3B), confirming the inbuilt adjuvant activity of the VLPs⁴⁰.

Next, Applicant produced slow-release implants (80% PLGA, 10% VLPs and 10% PEG8000) containing either unmodified Qβ (control) or QβS100A9 VLPs. Melt extrusion yielded 0.5×70 mm rods. The implants contained 300 μg of VLPs to match the combined dose of the prime plus two boosts schedule (FIG. 3E). As expected, the IgG titers against the S100A9 epitope were significantly higher in the PLGA/QβS100A9 VLP group than the control VLP group over a period of 20 weeks (FIG. 3F). The IgG titers in the PLGA/QβS100A9 implant VLP group were similar to those in the soluble QβS100A9 VLP group (˜104), confirming that a single dose of the PLGA/QβS100A9 implant is effective (FIG. 3B, FIG. 3F). From a clinical perspective, the single-dose implant favors patient compliance and reduces the healthcare burden by offering the potential for long-acting vaccines whose release rate can be tailored based on the polymer composition⁴¹.

Applicant next evaluated the immunoglobulin subtypes elicited by the vaccine formulations. IgG2b is a subclass of IgG induced primarily by Th1-type cytokines such as interferon gamma (IFN-γ), whereas IgG1 antibodies are induced by Th2-type cytokines such as IL-4^{40, 42}. The soluble VLPs resulted in an IgG1/IgG2b ratio<1 that persisted over 12 weeks, indicating a Th1-biased profile (FIG. 3D). In contrast, the implant resulted in a marked Th1 bias initially, but this trended toward a balanced Th1/Th2 response by week 15 (FIG. 3H). Applicant also observed that IgM titers declined over time following vaccination with soluble VLPs but increased over time following vaccination with the implant, indicating that the slow release maintains a constant boosting effect that influences the levels of IgM and other immunoglobulins (FIG. 3C, FIG. 3G).

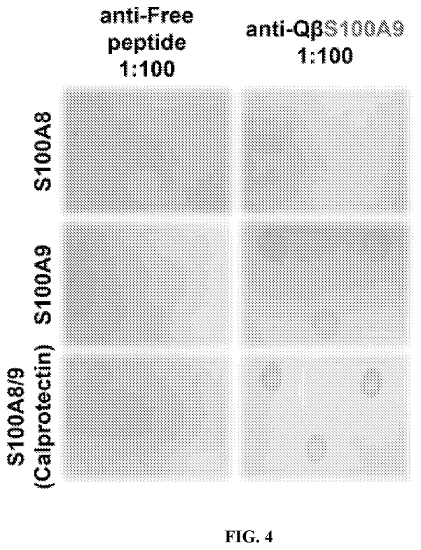

Given that S100A9 belongs to a large family of S100 proteins with similar structures and shared domains³⁶, Applicant selected an epitope unique to S100A9 to avoid off-target effects. To confirm the specificity of the antibodies elicited by the QβS100A9 vaccine, Applicant tested their ability to bind S100A8, S100A9 and S100A8/9 heterodimers in dot blot assays. As expected, plasma from QβS100A9-vaccinated mice only recognized S100A9 and the heterodimer S100A8/9, but not the S100A8 protein (FIG. 4). Antibodies from mice injected with the free peptide generated weaker signals but Applicant observed the same overall result (FIG. 4).

### Safety of Soluble QβS100A9 VLPs and Slow-Release Implants

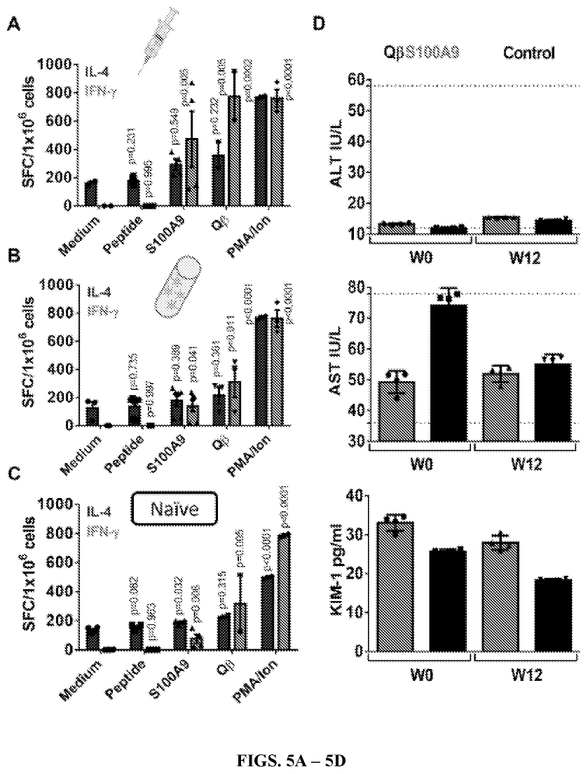

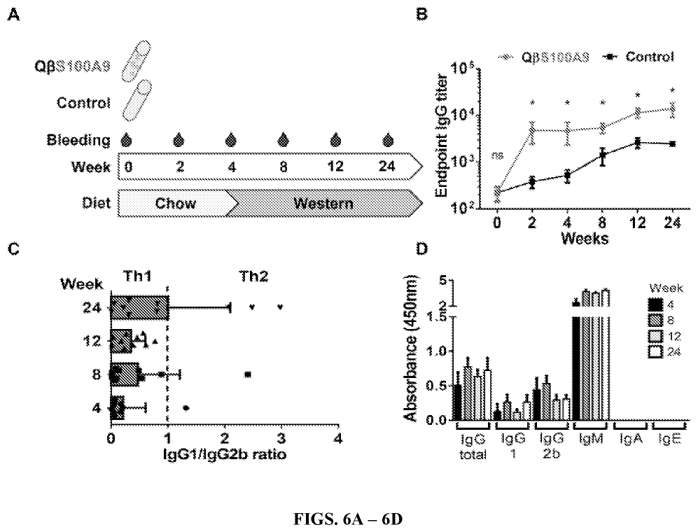

S100A9 is a self-antigen protein, so immunotoxicity parameters such as the absence of a target-specific T-cell response (Th and cytotoxic T cells) are desirable^{43, 44, 45} Applicant therefore used ELISpot assays to evaluate the activation of primed T cells following vaccination with soluble QβS100A9 VLPs or the implant. Applicant monitored the production of IFN-γ (linked to Th1-biased profiles) and IL-4 (linked to Th2-biased profiles) in splenocytes isolated 4 weeks after the second 100-μg dose of soluble QβS100A9 or a single 200-μg dose of the QβS100A9 implant, and also in splenocytes from naïve mice. IFN-γ was significantly more abundant in the splenocytes of vaccinated (soluble or implant) and naïve mice following stimulation with recombinant mouse S100A9, but not following stimulation with the free peptide (FIGS. 5A-5C). The detection of this response even in naïve splenocytes indicates that the S100A9 monomer and S100A8/9 heterodimer act as damage associated molecular patterns (DAMPs) via their ability to bind the RAGE, TLR-4 and EMMPRIN receptors, leading to MAPK signaling and the translocation of NF-κB from the cytosol to the nucleus, triggering the activation of genes that control inflammation^{46, 47}. This result makes it difficult to determine whether the presence of T cells primed with the S100A9 epitope could exacerbate the T cell response because even naïve splenocytes are activated in the presence of S100A9. However, the IFN-γ SFCs were more abundant on splenocytes from mice vaccinated with the soluble VLPs, which agrees with the persistent Th1-biased profile revealed by the IgG1/IgG2b ratio (FIG. 3C, FIG. 3D). On the other hand, the IFN-γ SFCs observed on splenocytes from mice vaccinated with the implant (FIG. 5B) showed a response comparable to that of naïve splenocytes, reflecting the balanced Th1/Th2 profile (FIG. 3G, FIG. 3H).

The stimulation of splenocytes with unmodified Qβ VLPs resulted in a significant increase in IFN-γ compared to the non-stimulated control (medium only), indicating a strong response to the VLP scaffold^{27, 29}. The phorbol 12-myristate 13-acetate (PMA)/ionomycin positive control was the only stimulant that triggered the significant production of both IFN-γ and IL-4 by splenocytes from all vaccinated and naïve mice. Accordingly, Applicant decided to study the PLGA-based QβS100A9 implant in more detail, given its more balanced Th1/Th2 response and its IgG titers comparable to the soluble VLPs. Most Qβ VLP vaccines developed by us²⁷ and others⁴⁴ triggered a Th2-biased response, but this is likely to be epitope-dependent given the Th1-biased response reported for a Qβ VLP self-antigen vaccine against epitopes derived from different loops of the C3 domain of IgE that binds to the high-affinity FcERI receptor⁴⁸. The Th1-biased response was dependent on TLR-7 activation because TLR-7 knockout mice switched to a Th2-biased profile⁴⁸. Applicant found that the Th response can be modulated by delivering the vaccine as a slow-release implant, probably reflecting the constant release of small quantities of VLPs during implant biodegradation.

Applicant also assessed the safety of the vaccines by measuring the concentration of kidney injury molecule 1 (KIM-1), a transmembrane glycoprotein and biomarker of kidney injury⁴⁹, as well as the plasma activity of aspartate aminotransferase (AST) and alanine transaminase (ALT), which are biomarkers of liver injury⁵⁰. Applicant compared mice vaccinated with QβS100A9 VLPs vs controls after 12 weeks but observed no significant changes on the normal range⁵¹ of KIM-1 or AST/ALT activity (FIG. 5D). Applicant can therefore confirm that the QβS100A9 VLPs neither altered kidney physiology nor caused liver injury.

### Efficacy of Soluble QβS100A9 VLPs and Slow-Release Implants Against Atherosclerosis

Having confirmed the immunogenicity and safety of the QβS100A9 VLPs and implants in healthy animals, Applicant tested the efficacy of the QβS100A9 implant in ApoE^{-/-} mice fed on a high-fat western diet as a model of atherosclerosis. Applicant selected the implant because it can be administered as a single dose but it elicits antibody titers similar to the soluble VLPs while generating a more balanced Th1/Th2 profile. Mice were immunized once with the QβS100A9 implant or the unmodified Qβ implant as a control and were monitored for 24 weeks, the first 4 weeks on a regular diet before switching at the beginning of week 5 to the high-fat diet (FIG. 6A). S100A9-specific IgG appeared during week 2, and titers remained stable until week 8 and then increased slightly between weeks 8 and 12 (FIG. 6B). As expected, the QβS100A9 group elicited significantly higher titers than the control group at all time points. Applicant assessed the IgG1/IgG2b ratio and observed a similar profile to that observed for healthy mice. In more detail, Applicant observed a Th1-biased profile (IgG1/IgG2b<1) for all except one of the mice at week 4. Most profiles remained the same at week 8, except one mouse with a balanced Th1/Th2 profile and two trending toward a Th2-biased response. Finally, three of the mice showed a Th2-biased response by week 24, two showed a balanced Th1/Th2 response, and five retained their Th1-biased profile (FIG. 6C). Additionally, as observed in the healthy animals, IgM levels remained high throughout the experiment (FIG. 6D). These data confirmed that the QβS100A9 implant behaves similarly in healthy and atherosclerotic mice.

### The QβS100A9 Implant Ameliorates Aortic Lesions by Reducing the Plasma Levels of Calprotectin and Pro-Inflammatory Cytokines

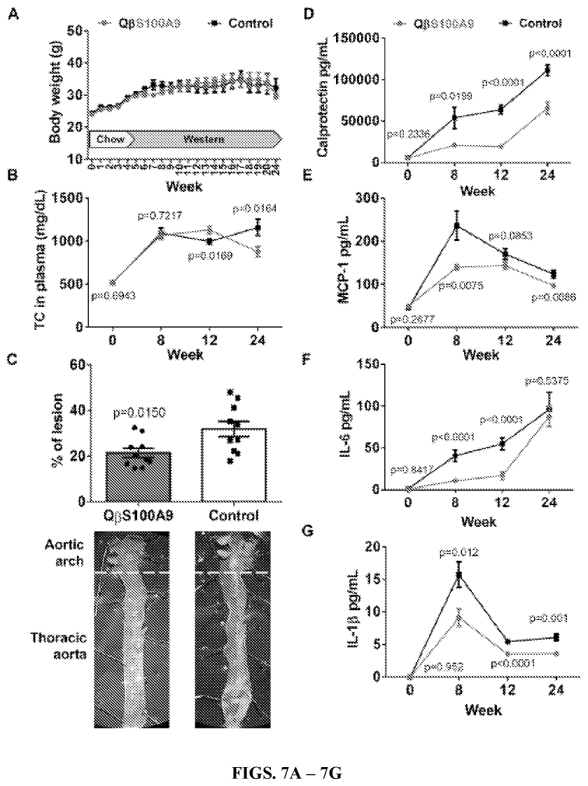

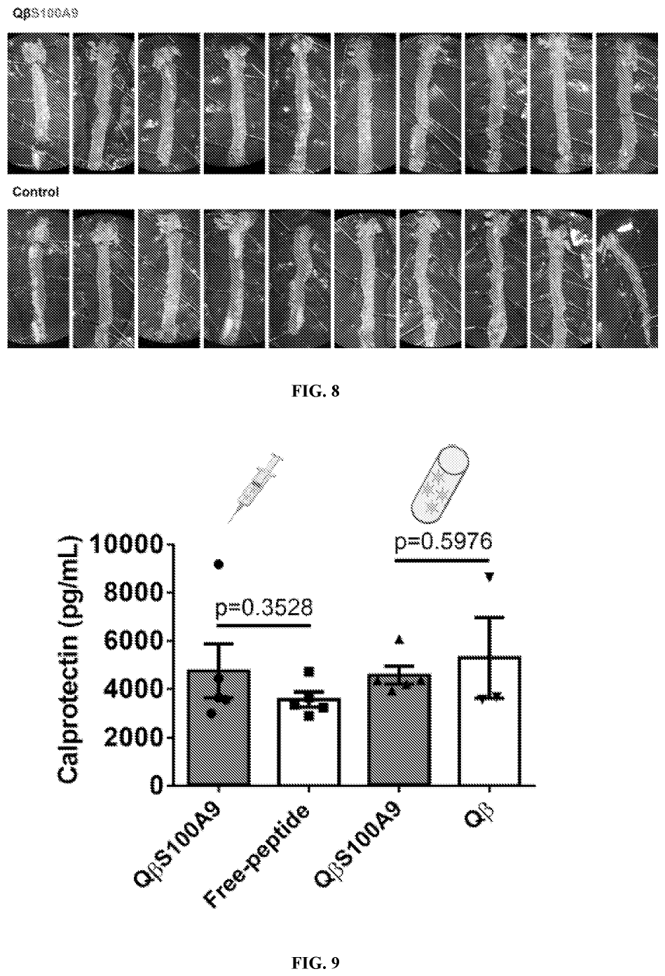

Applicant measured the degree of plaque formation on the aortas of mice fed on the high-fat western diet by standard oil red O staining, which detects neutral fat, fatty acids and triglycerides⁵² that accumulate in atherosclerotic plaques but not in the healthy endothelium. The positive staining is directly correlated to atherosclerosis and can be quantified as the percentage of lesion in the aortas (FIG. 7C). Applicant found that the percentage of lesion in the QβS100A9 vaccine implant group (21.5±2%) was 32% lower than that in the control group (31.6±3%). This is noteworthy because double knockout animals (ApoE^{-/-}S100A9^{-/-}) fed on a high-fat diet showed a 25-30% reduction in lesion area in the thoracic and abdominal aorta¹⁸, suggesting that blocking S100A9 activity either by gene knockout or vaccination may reduce the level of S100A9 in the plasma, thus preventing calprotectin from signaling via TLR-453 and modulating calcium signaling¹⁹, reorganizing the cytoskeleton⁵⁴ and triggering the release of proinflammatory cytokines.

To determine the mechanism of action, Applicant measured the levels of calprotectin in the plasma. Applicant found significantly lower levels in the QβS100A9 group compared to controls at all time points, reaching a maximum differential of 3.3-fold during week 12 (FIG. 7D). In addition to atherosclerosis and CVD^{13, 14, 17, 55}, calprotectin is an important biomarker for the diagnosis and monitoring of many other inflammatory diseases⁸ because it stimulates the production of pro-inflammatory cytokines/chemokines such as IL-1β56, IL-6 and MCP-157. IL-1β is key mediator of the pro-inflammatory response. Higher levels of this cytokine are found in atherosclerotic human arteries compared to healthy controls and its abundance positively correlates with disease severity⁵⁸. IL-1β activates secondary inflammatory mediators such as IL-6 and triggers pro-coagulant activity, the expression of adhesion molecules required for leukocyte recruitment, and the production of MCP-1^{59, 60} Together these changes promote the recruitment of monocytic phagocytes, which are strongly implicated in atherogenesis⁶¹. IL-1β deficiency attenuated the spontaneous development of atherosclerotic lesions in ApoE^{-/-} mice⁶². Applicant found that the lower levels of calprotectin in the QβS100A9 group correlated with lower levels of IL-1β, IL-6 and MCP-1 (FIGS. 7E-7G). These cytokines/chemokines were also depleted in ApoE^{-/-} S100A9^{-/-} double knockout mice, correlating with the less severe aortic lesions¹⁸.

The precise mechanism by which the S100A9-specific antibodies reduce the severity of lesions is unclear. When a similar epitope was used to treat thrombosis in mice (₁₀₄GHSHGKGCGK₁₁₃) (SEQ ID NO: 6), from mouse S100A9) and monkeys (₁₀₂GHHHKPGLGE₁₁₁) (SEQ ID NO: 7), from monkey S100A9), the promising results were attributed to the inhibition of S100A9/CD36 signaling in platelets^{36, 63}. Many pro-inflammatory cytokines are triggered by S100A9/TLR-4 signaling, but a recent study showed that 73-85 amino acids in S100A9 specifically interact with TLR-4 and trigger TNF-α expression⁵³. The data shows that the depletion of calprotectin is sufficient to reduce the secretion of pro-inflammatory cytokines/chemokines and is therefore protective against aortic lesions, without knowing which S100A9-dependent signaling pathway is blocked by the antibodies.

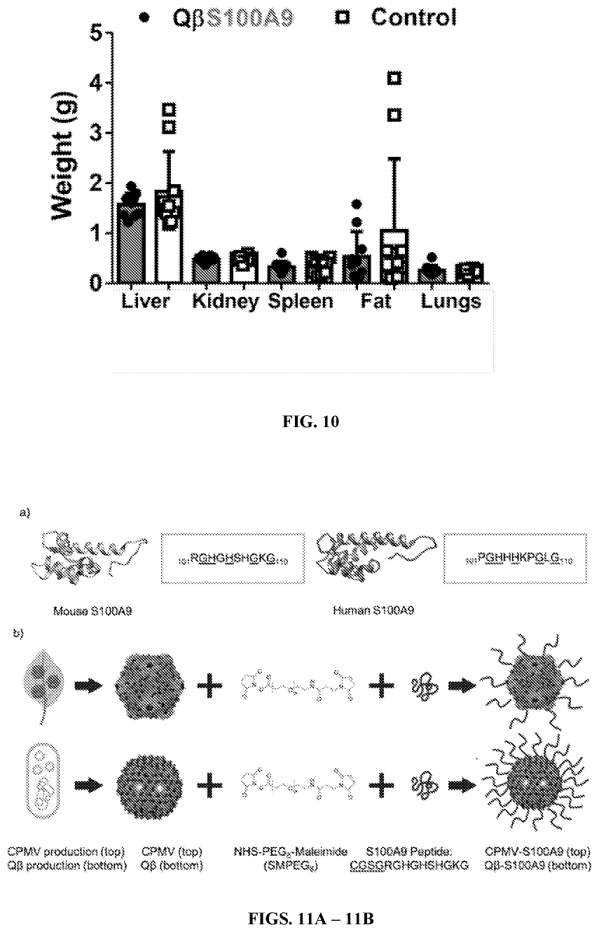

Interestingly, the QβS100A9 implant did not affect plasma levels of calprotectin in healthy animals on a regular diet (FIG. 9) but prevented the levels from increasing when these animals were fed on the high-fat diet (FIG. 7D). This is important because calprotectin also protects against pathogens⁶⁴, and it would be undesirable to completely remove a beneficial defense mechanism. Accordingly, Applicant measured other health-related parameters beyond the severity of aortic lesions and cytokine levels. Applicant observed no significant differences in body weight between the treatment groups at any point during the experiment, which was anticipated because the vaccine does not affect food intake (FIG. 7A). However, around the middle of the study (week 12) Applicant observed a significantly higher amount of total cholesterol in the QβS100A9 vaccine group (1130±44 mg/dL) compared to the control (996±31 mg/dL), but this relationship switched by week 24, with the QβS100A9 vaccine group (878±60 mg/dL) showing a significantly lower amount of total cholesterol than the controls (1158±94 mg/dL) (FIG. 7B). The relationship between systemic S100A9 and cholesterol homeostasis is unclear, and would be an important subject for further investigation. Applicant found no significant difference in the weights of individual organs between the QβS100A9 vaccine group and controls (FIG. 10).

In summary, the single-dose QβS100A9 vaccine formulated as a PLGA:VLP implant displaying the C-terminal peptide of S100A9 successfully reduced the extent of aortic lesions in a model of atherosclerosis, most likely by depleting calprotectin and thus preventing the secretion of pro-inflammatory cytokines/chemokines such as IL-1β, IL-6 and MCP-1. This was achieved without apparent systemic damage or adverse autoimmune responses. Calprotectin remained at normal basal levels in healthy mice. The effect of the vaccine was to deplete calprotectin only in the disease state when unvaccinated animals experienced elevated levels. This promising anti-atherosclerosis vaccine offers a novel approach for the management of CVD and other inflammatory diseases.

### Methods

### Production of Qβ VLPs.

Bacteriophage Qβ VLPs were expressed as previously reported^{27, 29, 65} Genes encoding the wild-type Qβ CP (NCBI accession: P03615) and CP fused to the mouse S100A9 epitope ₁₀₁RGHGHSHGKG₁₁₀ (SEQ ID NO: 2) (NCBI accession: P31725) were codon optimized for E. coli and inserted into the expression vector pCOLA-DUET1 by GenScript Biotech. A linker (GSG) was placed between the C-terminus of the CP and the N-terminus of the peptide. The final vector was named pCOLA_Qβ_QβS100A9. As a control for some experiments, Applicant used vector pCDF_Qβ carrying only the wildtype Qβ CP gene²⁷. E. coli B121 (DE3) cells (New England BioLabs) transformed with pCDF_Qβ or pCOLA_Qβ_QβS100A9 were grown at 37° C. for 16 h shaking at 250 rpm in 10 mL MagicMedia (Invitrogen) with the appropriate antibiotics: 25 μg/mL streptomycin (Sigma-Aldrich) for pCDF_Qß and 50 μg/mL kanamycin (Sigma-Aldrich) for pCOLA_Qβ_QβS100A9. The culture was scaled up to 200 mL in the same medium and incubated at 37° C. for 20 h, shaking at 300 rpm. The cells were pelleted by centrifugation (5000×g, 20 min, 4° C.) and frozen at −80° C. overnight. The pellet was then lysed by resuspending it in 10 mL lysis buffer (GoldBio) per gram of wet mass, adding a lysis cocktail comprising 1 mg/mL lysozyme (GoldBio), 2 μg/mL DNase (Promega) and 2 mM MgCl2, and incubating at 37° C. for 1 h before sonicating at 30% amplitude for 10 min on ice, with 5-s pulses interspersed with 5-s gaps. The lysate was centrifuged (5000×g, 30 min, 4° C.) and the clear supernatant was set aside. Unmodified Qβ VLPs and hybrid QβS100A9 VLPs were precipitated by adding 10% (w/v) PEG8000 (Thermo Fisher Scientific) at 4° C. for 12 h. The precipitated fraction was pelleted by centrifugation (5000× g, 10 min, 4° C.) and dissolved in phosphate-buffered saline (PBS; 137 mM NaCl, 2.7 mM KCl, 10 mM Na2HPO4, 1.8 mM KH2PO4, pH 7.4) and then extracted with 0.5 volumes of 1:1 (v/v) butanol/chloroform. The aqueous fraction containing VLPs was separated by centrifugation (5000× g, 10 min, 4° C.) and pure VLPs were recovered by 10-40% sucrose velocity gradient ultracentrifugation (9,6281×g, 2.5 h, 4° C.). The light-scattering VLP band was collected and pelleted by ultracentrifugation (16,0326×g, 2 h, 4° C.) and the pure VLPs were resuspended in PBS and stored at 4° C. until further use.

### Characterization of Qβ VLPs.

The VLPs were characterized as previously described^{27, 28, 29}. The VLP concentration was determined using a Pierce BCA assay kit (Thermo Fisher Scientific). To confirm hybrid QβS100A9 assembly and peptide display, 10 μg of QβS100A9 particles was analyzed by SDS-PAGE under reducing conditions on NuPAGE 12% Bis-Tris protein gels (Thermo Fisher Scientific) stained with GelCode Blue Safe protein stain (Thermo Fisher Scientific). The gel images were acquired using the ProteinSimple FluorChem R imaging system, and densitometry was used to determine the number of peptides displayed per hybrid QβS100A9 VLP. The integrity of VLPs was confirmed by TEM using a FEI Tecnai Spirit G2 BioTWIN instrument to examine samples stained with 2% uranyl acetate. FPLC was carried out using an AKTA-FPLC 900 system fitted with Superose 6 Increase 10/300 GL columns (GE Healthcare) using PBS as the mobile phase. Particle size was confirmed by DLS using a Malvern Instruments Zetasizer Nano at 25° C. and plastic disposable cuvettes.

### Hot-Melt Extrusion of VLP-Loaded Implants.

Implants were prepared from PLGA (Akina; LG ratio=50:50, molecular weight=10-15 kDa) using Applicant's previously reported desktop melt-processing system^{27, 28, 29, 32, 33, 34}. Briefly, lyophilized QβS100A9 or Qβ VLPs were formulated with the ratio 80% PLGA, 10% VLP and 10% PEG8000 (by weight). The dry components were vortexed and loaded into the hot melt-processing system. The barrel was heated to 70° C. for 90 s, and the piston was set to 10 psi (69 kPa) for extrusion. Implants were stored at room temperature with a desiccant until use. Immunization of mice. All animal experiments were approved by the UC San Diego Institutional Animal Care and Use Committee (assurance number D16-00020, protocol number S18021). Eight-week-old female C57BL/6J mice (Jackson Laboratory, #000664) were kept under controlled conditions with food and water provided ad libitum. Five animals were assigned to the following four groups: (1) soluble QβS100A9, (2) soluble free peptide (control), (3) QβS100A9 implant, and (4) Qβ implant (control). For soluble formulations, Applicant injected 100 μg of QβS100A9 VLPs or 5 μg of free peptide epitope (synthesized by GenScript Biotech) s.c. in 100 μl of PBS every 2 weeks, making three doses in total (prime+two boosts). The PLGA-based implants were cut into lengths of 0.3-0.5 cm according to their weight to provide 300 μg of QβS100A9 vaccine (to match the 300 μg total dose of the soluble formulation) or unmodified Qβ as control. Implants were placed using an 18 G needle (BD Biosciences) s.c. behind the neck. Blood samples were taken by tail bleeding at week 0 (before vaccination) and at multiple time points thereafter. Plasma was separated in lithium/heparin-treated tubes (Thomas Scientific) by centrifugation (2000×g, 10 min, room temperature) and the plasma was stored at −80° C.

### Peptide-Specific IgG Titers.

Endpoint total IgG titers against the S100A9 peptide epitope were determined by enzyme-linked immunosorbent assay (ELISA) 27, 29, 34. Applicant coated 96-well, maleimide-activated plates (Thermo Fisher Scientific) with 2.5 μg cysteine-modified S100A9 peptide (CGSGRGHGHSHGKG) (SEQ ID NO: 8) in 100 μL coating buffer per well (0.1 M sodium phosphate, 0.15 M sodium chloride, 10 mM EDTA, pH 7.2) overnight at 4° C. After washing (3× 5 min) with 200 μL/well PBS+0.05% (v/v) Tween-20 (PBST), the plates were blocked for 1 h with 1% (w/v) L-cysteine (Sigma-Aldrich). After washing as above, plasma samples from vaccinated animals (two-fold serial dilutions in coating buffer) were added to the plates and incubated for 1 h at room temperature. After washing as above, Applicant added a horseradish peroxidase (HRP)-labeled goat antimouse IgG (H+L) secondary antibody (Thermo Fisher Scientific, A16072) diluted 1:5000 in PBST (100 μL/well) and incubated as above. Finally, Applicant added 1-Step Ultra TMB substrate (Thermo Fisher Scientific, 100 μL/well) and stopped the reaction after 5 mins with 100 μL/well 2 M H₂SO₄. The endpoint IgG titers were defined as the reciprocal plasma dilution at which the absorbance at 450 nm exceeded twice the background value (blank wells without a plasma sample).

### Igg Subclasses and Immunoglobulin Isotypes.

Pooled samples from weeks 2, 8 and 12 (soluble vaccine) or 5, 10 and 15 (implant vaccine) were diluted 1:1000 in coating buffer and tested as described for the endpoint IgG titers above, but the secondary antibodies were HRP-labeled goat anti-mouse IgG1 (Invitrogen PA174421, diluted 1:5000), IgG2b (Abcam ab97250, diluted 1:5000), IgA (Abcam ab98708, diluted 1:5000), IgE (Invitrogen PA184764, diluted 1:1000), and IgM (Abcam ab97230, diluted 1:5000). The IgG1/IgG2b ratio was used to define the response as Th2-biased (IgG1/IgG2b>1) or Th1-biased (IgG1/IgG2b<1).

### ELISpot Assay.

The ELISpot assay was carried out using a mouse IFN-γ/IL-4 doublecolor ELISPOT kit (Cellular Technology) 27, 29, 34 Briefly, 96-well ELISpot plates were coated with anti-mouse IFN-γ and anti-mouse IL-4 antibodies overnight at 4° C. Splenocyte suspensions (1× 106 cells/well) collected from n=3 mice 4 weeks postimmunization or n=2 naïve mice were cultured with 100 μL medium alone (negative control), 10 μg free S100A9 peptide, 5 μg recombinant mouse S100A9 protein (R&D Systems), 5 μg unmodified Qβ, or 5 ng PMA/100 ng ionomycin (Sigma-Aldrich, positive control) at 37° C. in a 5% CO₂ atmosphere for 24 h. After washing with PBST, the plates were incubated with fluorescein isothiocyanate (FITC)-labeled anti-mouse IFN-γ (1:1000 dilution) and biotin-labeled anti-mouse IL-4 (1:666 dilution) antibodies for 2 h at room temperature. After washing as above, Applicant added streptavidin alkaline phosphatase (AP, 1:1000 dilution) and anti-FITC-HRP (1:1000 dilution) to each well and incubated for 1 h at room temperature. After a final wash with PBST and a rinse in distilled water, Applicant added the AP substrate and incubated for 15 min at room temperature, then rinsed with distilled water, added the HRP substrate, and incubated for 10 min at room temperature. The plates were rinsed five times with distilled water and air-dried at room temperature overnight. Colored spots were quantified using the Immunospot S6 Entry analyzer. Splenocytes were evaluated per animal and tested at least in duplicate for each stimulant. The results were reported as spot-forming cells (SFC) per 1×10⁶ cells.

### Dot Blot Assay.

The C-terminal S100A9 epitope is unique among the S100 family⁶⁶ so Applicant used a dot blot assay to confirm the specificity of the antibodies elicited by QβS100A9 particles. Applicant spotted 2 μL (1 μg) of recombinant mouse S100A8, S100A9 or heterodimer S100A8/9 (R&D Systems) onto a nitrocellulose membrane (0.45-μm pore size, GE Healthcare) and blocked with 3% bovine serum albumin (Roche) at room temperature for 1 h, followed by washing with PBST. The membrane was then incubated at room temperature for 1 h with pooled plasma (week 4) from QβS100A9-vaccinated or free-peptide-vaccinated mice (diluted 1:100 in PBS). After another wash with PBST, Applicant incubated the membrane with HRP-labeled goat anti-mouse IgG (diluted 1:5000 in PBST) at room temperature for 1 h. Finally, the membrane was washed as above and incubated with 3,3′-diaminobenzidine (DAB) substrate for 1 min. The development of a brown color indicated specific binding to the S100 proteins.

### Liver and Kidney Biomarkers.

The safety of the QβS100A9 vaccine was determined by detecting plasma biomarkers related to liver and kidney injury. For liver damage, Applicant determined the concentrations of the enzymes AST and ALT using the corresponding activity assay kits (Abcam). For kidney damage, Applicant determined the concentration of KIM-1 using the Mouse KIM-1 ELISA Kit (Abcam). Plasma samples collected and tested at weeks 0 and 12 post-vaccination. The plasma samples were pooled from each group and tested in quadruplicate.

### Mouse Atherosclerosis Model.

To determine the efficacy of the disclosed vaccine candidates, they were tested in the ApoE^{-/-} mouse model of atherosclerosis (Jackson Laboratory, #002052). Eight-week-old male ApoE^{-/-} mice were fed on the Envigo TD.88137a western purified atherogenic diet (20-23% milkfat/butterfat, 0.2% total cholesterol, 34% sucrose by weight). The animals were vaccinated as described above with PLGA-based QβS100A9 implants containing 300 μg VLPs, and matching implants containing the same dose of unmodified Qβ VLPs as controls. The mice were fed on a regular diet for 4 weeks post-immunization before switching to the high-fat diet for 20 weeks. Mice were fasted for 4 h and blood was sampled at weeks 0, 2, 4, 8, 12 and 24. Plasma was separated to determine endpoint IgG titers and immunoglobulin isotypes as described above. At the end of the study (week 24), the mice were euthanized by CO2 asphyxiation followed by exsanguination by cardiac puncture. Aortas were perfused with PBS (pH 7.4) before the thoracic aorta and aortic arch were dissected, fixed with 4% formalin, denuded of connective tissue and processed for oil red O staining. Hearts, livers, kidneys, lungs, spleens, and abdominal fat tissues were weighed and fixed with 4% formalin and embedded in paraffin for further analysis.

### Oil Red O Staining.

Aortas were stained as previously reported⁶⁷. Briefly, cleaned and fixed aortas were placed individually in 1.5-mL tubes and equilibrated in 1 mL 78% methanol by gentle motion on a tilted roller (2× 5 min). The methanol was then replaced with 1 mL fresh 0.2% oil red O solution and the tissue was incubated at room temperature for 1 h. After staining, the tissue was transferred to a clean tube and washed with 1 mL 78% methanol on the tilted roller (2×5 min). Finally, the methanol was replaced with 1 mL PBS and aortas were stored at 4° C. To visualize and quantify the atherosclerotic lesions, aortas were dissected longitudinally and pinned with the lumen siding facing up on a dissecting dish under a stereomicroscope (Vista Vision, 10X). The percentage of atherosclerotic lesions was determined by densitometry using ImageJ v1.44o (http://imagej.nih.gov/ij) and was calculated as follows: % lesion=(total lesion areas [stained red area]/total aorta area)*100.

### Cytokine Quantification.

Commercial sandwich ELISA kits were used to measure the concentrations of different cytokines in plasma from QβS100A9-vaccinated and control mice. Applicant used kits for mouse calprotectin S100A8/9 heterodimer (R&D Systems, DY8596-05), mouse MCP-1 (Abcam, ab208979), mouse IL-1β (Abcam, ab229440) and mouse IL-6 (R&D Systems, M6000B).

### Statistical Analysis.

Data are presented as means±standard errors of the mean (SEM), with the number of replicates presented for individual experiments. Differences between groups were analyzed using an unpaired two-tailed t-test. Data were analyzed in GraphPad Prism v6, with p<0.05 defined as the threshold for statistical significance.

### Experiment No. 2:

### Production and Characterization of S100A9 Vaccine Candidates.

A recent study reported an S100A9 vaccine targeting the C-terminal of S100A9 104GHSHGKGCGK₁₁₃⁴⁰ (SEQ ID NO: 6). Applicant modified the vaccine (₁₀₁RGHGHSHGKG₁₁₀) (SEQ ID NO: 2) to improve the sequence to be above the 0.5 threshold for a B-cell epitope according to the BepiPred-2.0 Sequential B-Cell Epitope Predictor (41) (FIG. 11A). Extensive biochemical characterization has confirmed that murine S100A9 is functionally equivalent to its human counterpart⁴² with sequence similarity and identity of 79 and 62%, respectively (following NCBI Blast alignment of GenBank peptide sequences of CAC14292 and NP_002956 of mouse and human S100A9, respectively). Nevertheless, amino acid differences in the peptide may require optimization for human clinical translation. Applicant additionally added a GSG linker to the peptide improve peptide flexibility and thereby peptide conjugation efficiency.

To create the S100A9 vaccine candidates, CPMV was harvested from black-eyed pea No. 5 plants, and Qβ VLPs were expressed in BI21 (DE3) E. coli as previously reported^{38, 43} CPMV and Qβ present solvent-exposed surface lysines (Lys) (300 per CPMV and 720 per Qβ VLP), which are conjugated to an SM(PEG)8 linker followed by conjugation to the cysteine-terminated S100A9 peptide (FIG. 11B) 44, 45.

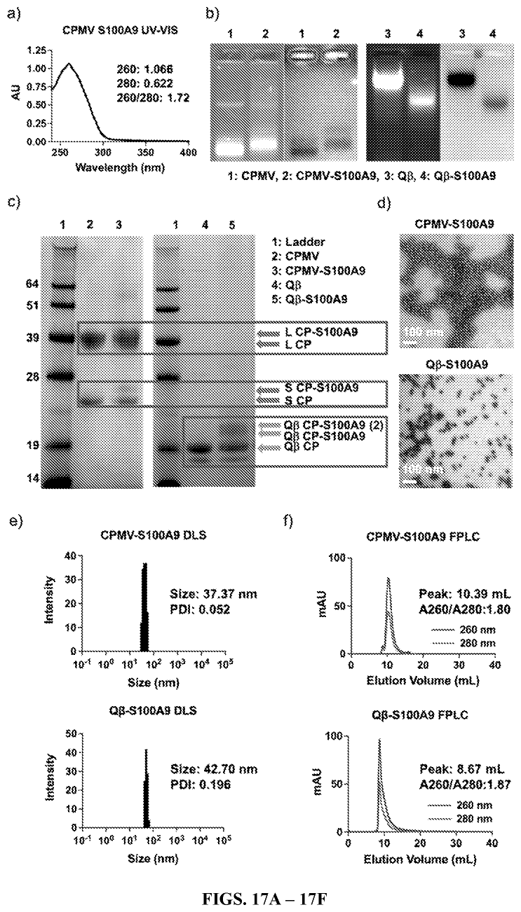

Vaccine conjugates denoted as CPMV-S100A9 and Qβ-S100A9 were purified and characterized by ultraviolet-visible spectroscopy (UV-VIS) (FIG. 17A) or bicinchoninic acid (BCA) assay to determine their concentration and purity. The yields following conjugation and purification were ˜50% of starting material. To confirm structural integrity and epitope display, native gel electrophoresis (FIG. 17B) as well as sodium dodecyl sulfate-polyacrylamide gel electrophoresis (SDS-PAGE, FIG. 17C) was performed. While CPMV-S100A9 had reduced electrophoretic mobility vs. CPMV, Qβ-S100A9 showed increased mobility vs. Qβ. In both the CPMV and Qβ gels, the RNA and protein co-localize indicating that stable and intact conjugates were obtained. While Qβ is a VLP, it packages host RNA, which is visualized by the GelRed stain⁴⁶. SDS-PAGE demonstrated conjugation of ˜120 peptides per Qβ and ˜ 30 peptides per CPMV (FIG. 17C), consistent with a higher density of surface Lys per Qβ vs. CPMV^{44, 45}. CPMV consists of 60 copies each of two coat proteins (CPs), a large (L) CP of 42 kDa and a small(S) CP of 24 kDa (FIG. 17A, red boxes). The secondary, higher molecular weight band above the native CP bands (e.g., S CP-S100A9 or L CP-S100A9 as well as Qβ CP) is in agreement with coupling of the S100A9 peptide, which has a molecular weight of 1.33 kDa (including the linker). Quantification of peptide per nanoparticle and ratio of CP-S100A9 vs. free CP was carried out using densitometry analysis and ImageJ software (imagej.nih.gov/ij/download.html) to analyze the ratio of CP-S100A9 vs. free CP.

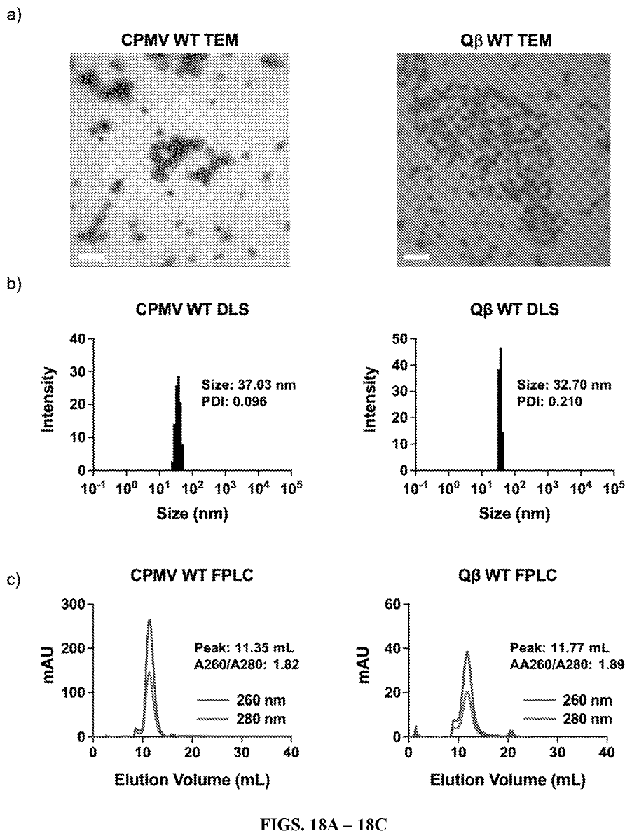

Transmission electron microscopy (TEM), dynamic light scattering (DLS), and size exclusion chromatography (SEC) using fast protein liquid chromatography (FPLC) of the vaccine candidates confirmed presence of intact particles (FIGS. 17D-17F). The TEM images showed monodisperse and intact nanoparticles with similar size and morphology to the native particles (FIG. 18A). The DLS data and SEC analysis corroborate these results (FIG. 17E, FIG. 17F, FIG. 18B, FIG. 18C). The elution profiles of CPMV-S100A9 and CPMV were similar around 11 mL and an A260:280 ratio of 1.8, indicating intact particles (FIG. 17F, FIG. 18C). For the Qβ-S100A9 sample, the size did increase after conjugation with ˜32.7 for Qβ and ˜42.7 nm for Qβ-S100A9 as well as elution profiles of 8.7 mL for Qβ-S100A9 vs. 11.8 mL for Qβ (FIG. 17E, FIG. 18B).

Immunization with CPMV-S100A9 and Qβ-S100A9 Vaccine Candidates.

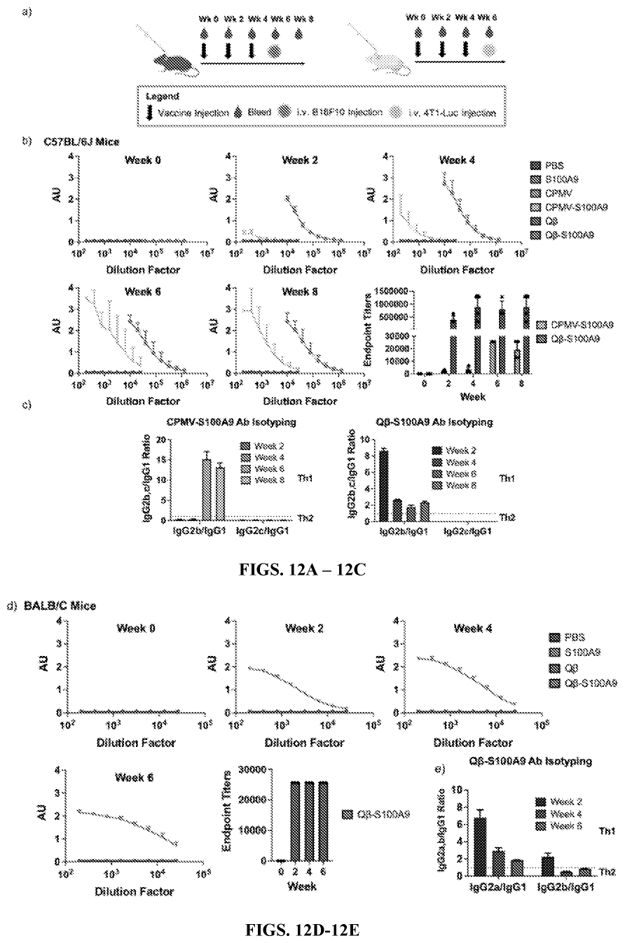

C57BL/6J mice were vaccinated 3× spaced two weeks apart (FIG. 12A). The CPMV-S100A9 and Qβ-S100A9 vaccines were injected subcutaneously (s.c.) at 200 μg per dose along with negative controls of PBS, CPMV, Qβ, and the S100A9 peptide (free S100A9 dose was equilibrated to the Qβ-S100A9 sample as the Qβ-S100A9 sample contained 4× more peptide than CPMV-S100A9). Pre- and post-immunization bleeds were collected and two weeks after the last boost, tumor challenge was performed (see below). Both CPMV-S100A9 and Qβ-S100A9 elicited significant antibody titers against the S100A9 peptide, as measured through enzyme linked immunosorbent assay (ELISA) (FIG. 12B). CPMV-S100A9 endpoint titers peaked at week 6 at 25,600 while Qβ-S100A9 end point titers remained consistent from weeks 4 to 8 ranging from 800,000 to 880,000. The higher antibody titers of the Qβ-S100A9 vs. CPMV-S100A9 are explained by the higher density of labeling of Qβ-S100A9. As expected, the control groups did not yield any S100A9-specific antibodies. Importantly, the lack of antibody production by the peptide group demonstrates that peptide vaccination is ineffective and that a necessary adjuvant and carrier such as with CPMV or Qβ is necessary. Applicant further investigated the IgG isotypes. A ratio of IgG2b IgG1⁻¹>1 was deemed as a Th1 bias while a ratio<1 was deemed to be Th2-biased⁴⁷. In the C57BL/6J mice, Applicant saw opposing trends in the isotyping patterns between the vaccines (FIG. 12C). While CPMV-S100A9 originally started with a Th2 bias, by week 6, the bias began to skew strongly towards Th1. The strong delineation towards a Th1 bias may be due to the adjuvant nature of CPMV-CPMV leads to upregulation of Th1 cytokines such as interferon (IFN)γ⁴⁸. On the other hand, vaccination with Qβ-S100A9 started Th1, but appeared balanced starting from week 4. Because C57BL/6J mice also produce IgG2c antibodies, the ratio of IgG2c to IgG1 was also investigated, but IgG2c antibodies were not produced with either vaccine (FIG. 12C).

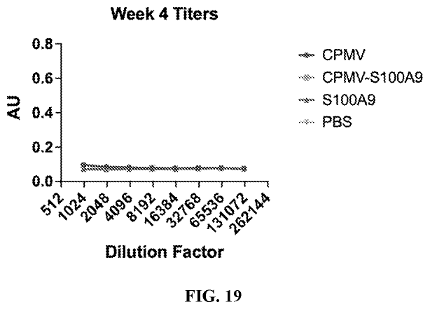

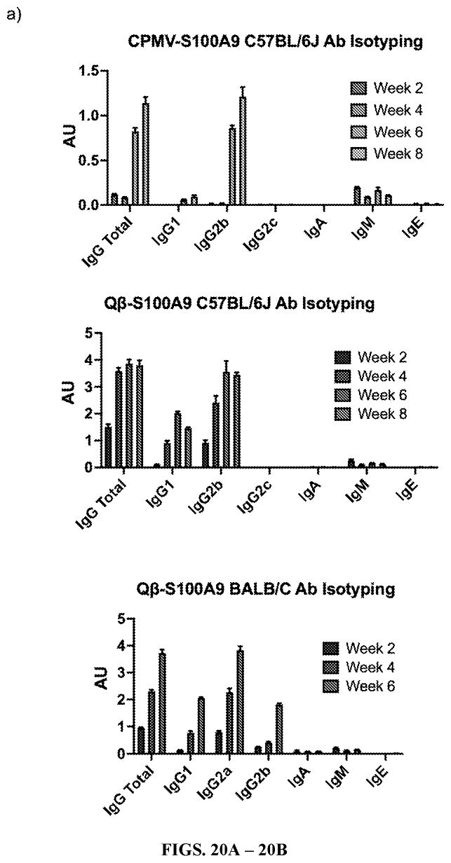

Mice immunizations were repeated in BALB/C mice. The CPMV-S100A9 group did not elicit significant antibody titers (FIG. 19); in contrast, Qβ-S100A9 produced strong titers starting from week 2 (FIG. 12D). Immune responses in BALB/C are Th2-biased, while C57BL/6J exhibit Th1 bias⁴⁹. CPMV is known to induce a strong Th1 response; therefore, as an adjuvant, it may not be as potent in the Th2-biased BALB/C mice. The isotyping was repeated in the BALB/C mice with the Qβ-S100A9 formulation (FIG. 12E). The Qβ-S100A9 initially produced a Th1 bias and then regressed to a balanced response as in the C57BL/6J mice (FIG. 12C, FIG. 12E). BALB/C mice produce IgG2a antibodies, so the IgG2a IgG1-1 ratio was determined and showed similar trends-starting with a Th1 bias and then shifting towards a balanced response. Lastly, IgM antibodies were measurable in both vaccine groups in BALB/C and C57BL/6J mice, but IgE was not apparent (FIG. 20A). IgA was detectable in small quantities in BALB/C mice only for the Qβ-S100A9 group (FIG. 20B). The immunization with CPMV/Qβ-S100A9 elicited high antibody titers; the viral nanoparticles act as a carrier and adjuvant, and recent research highlights that in particular conjugation results in co-delivery of the epitope and adjuvant to the same cell boosting the potency of the antibody production⁵⁰⁻⁵³.

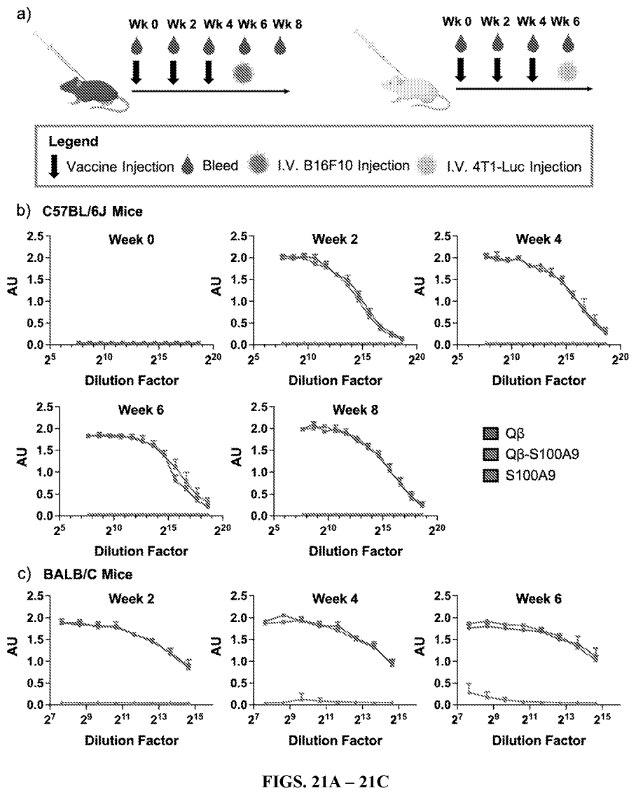

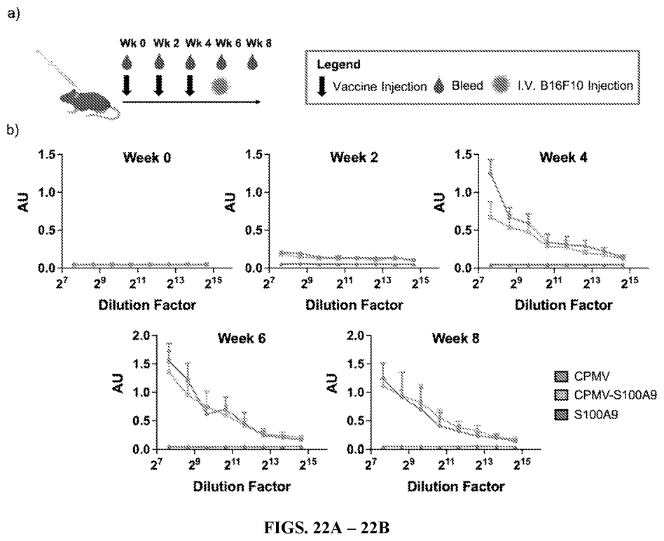

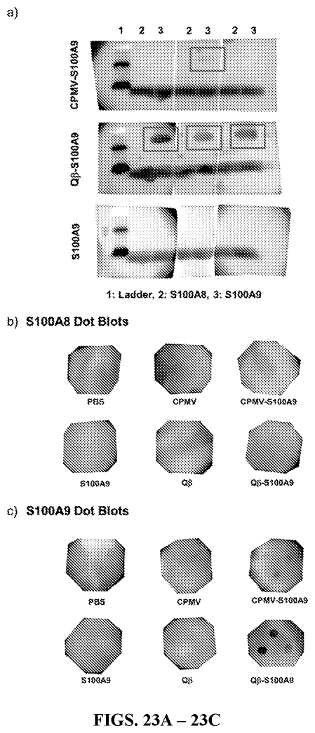

Antibody production against the CPMV and Qβ was also measured (FIG. 21, FIG. 16). In BALB/C and C57BL/6J mice, the Qβ endpoint titers were at 25,600 and 409,600 for all timepoints, respectively, which were the last dilutions tested (FIG. 21A, FIG. 21B). Applicant expected antibodies against the carrier and data suggests that presence of α-carrier antibodies does not impair vaccine efficacy³⁵. In fact, presence of α-carrier antibodies may boost efficacy—for example, in ongoing trials with a Qβ-based in situ immunotherapy, patients are pre-immunized with Qβ VLPs to stimulate α-Qβ antibody production, which enhances APC uptake and targeting⁵⁴⁻⁵⁶. CPMV and CPMV-S100A9 also induced potent antibody production against the CPMV viral nanoparticle (VNP) capsid (FIG. 22). At week 2, they both had average endpoint titers of 19,200 while at weeks 4-8, they both had endpoint titers of 25,600, the last measured dilution (FIG. 22). Next, western blots (WBs) and dot blots (DBs) were performed to validate whether antibodies produced recognize full-length S100A9 protein (denatured or native, respectively). WBs and DBs did indeed confirm binding to S100A9 with no cross-reactivity against another member of the S100A family, S100A8 (FIG. 23).

CPMV- and Qβ-S100A9 Vaccines Decrease Tumor Seeding within the Lungs of Melanoma and TNBC.

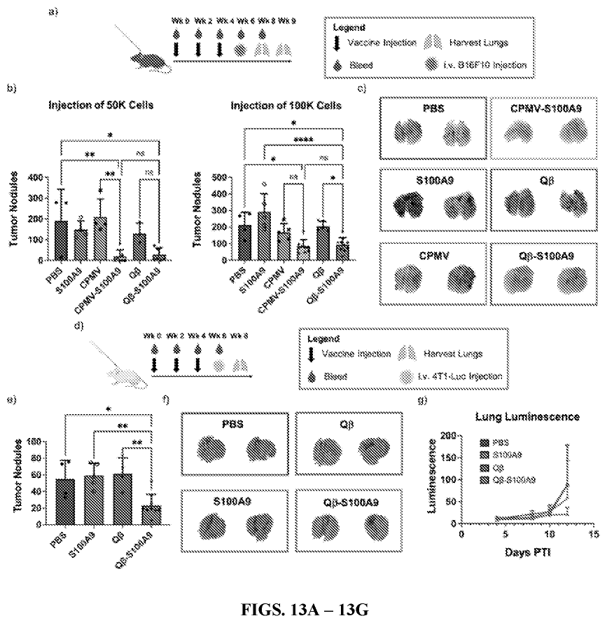

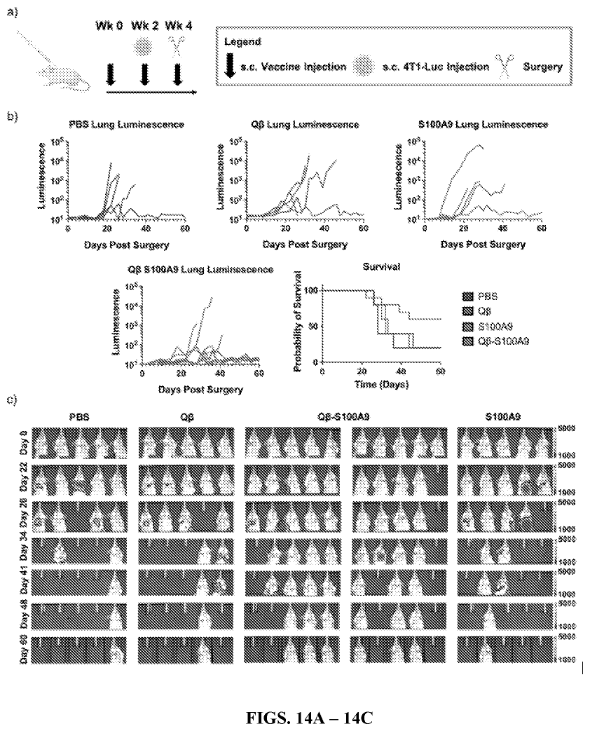

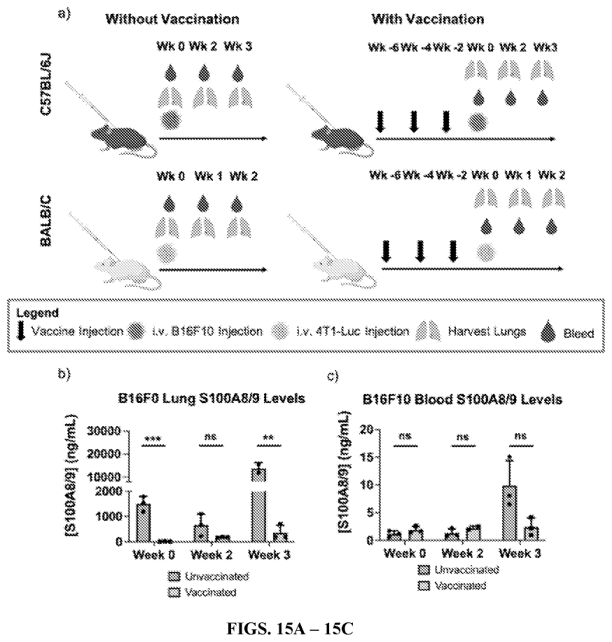

To assess the effectiveness of the vaccines in preventing metastasis, Applicant started with i.v. models of B16F10 melanoma and 4T1-Luc TNBC. While these models are not perfect indicators of metastasis, they can be used as tumor seeding models within the lungs. Applicant vaccinated both C57BL/6J and BALB/C mice prior to B16F10 and 4T1-Luc injection (FIG. 13A, FIG. 13D) simulating vaccination post-surgery before the seeding of metastatic nodules within the lungs. Three weeks post-B16F10 challenge (using 50,000 cells), the lungs were harvested and tumor nodules were manually counted. Compared to the control groups (CPMV, PBS, and free S100A9 peptide), the CPMV-S100A9 vaccine led to a 10.7-, 9.7-, and 7.6-fold reduction in tumor nodules, respectively (FIG. 13B, FIG. 13C). With the Qβ-S100A9 vaccine, there was a 4.5-, 6.6-, and 5.1-fold reduction in tumor nodules compared to Qβ, PBS, and the S100A9 peptide, respectively (FIG. 13B). It is of note that there was no significant difference between the Qβ-S100A9 and CPMV-S100A9 vaccines, which may indicate that a threshold of effective titers was achieved and that a lesser dose of Qβ-S100A9 may be sufficient. Applicant further validated vaccine efficacy by injecting 100,000 (vs. 50,000) B16F10 melanoma cells and harvesting the lungs after 2 weeks-again, potent efficacy was demonstrated (FIG. 13B, FIG. 13C).

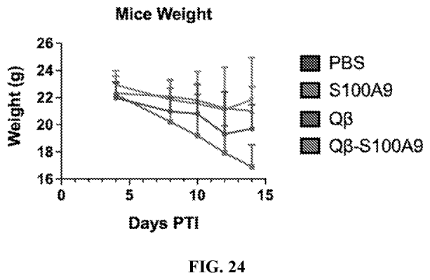

Next, Applicant investigated Qβ-S100A9 efficacy using a 4T1-Luc TNBC model in BALB/c mice. Lungs were harvested 2 weeks post-tumor challenge and tumor nodules were counted after staining with Bouin's solution (FIGS. 13D-13F). Again potent vaccine efficacy was observed decreasing tumor nodule counts by 2.6-, 2.4-, and 2.6-fold compared to Qβ, PBS, and S100A9 peptide, respectively (FIG. 13E, FIG. 13F). Weight measurements indicated no weight loss observed for the Qβ-S100A9 and S100A9 group; in contrast, PBS and Qβ mice lost weight drastically by day 8 (FIG. 24). Tumor growth was measured using luminescence measurements on an in vivo imaging system (IVIS) and averaged region of interest (ROI) counts on the Living Image 3.0 software. Qβ-S100A9 vaccinated mice showed the slowest tumor growth, and by day 12, the luminescence within the lungs of the Qβ-S100A9 mice was 24.7, 24.5, and 38% of the Qβ, PBS, and S100A9 controls, respectively (FIG. 13G). The results from both the B16F10 and 4T1-Luc studies demonstrate that the vaccine protects against tumor seeding within the lungs—in the clinic, Applicant envisions the vaccine working similarly in post-surgery patients with circulating tumor cells.

### Qβ-S100A9 Vaccine Decreases Metastasis to the Lungs in TNBC Surgery Study.

To investigate the ability of the vaccine in reducing metastasis, a s.c.-implanted 4T1-Luc primary tumor was surgically removed and metastasis to the lungs was examined. This experimental process mirrors clinical procedure, and it is known that 4T1-Luc cells begin to metastasize by day 1639. The treatment schedule is outlined in FIG. 14A. The lungs were imaged every two days following surgery, and the luminescence and survival of the mice were analyzed. In the control animals, tumors developed quickly following surgery, and by day 46, 80% of the mice in each group had died due to lung metastasis (FIG. 14B). On the other hand, 60% of the mice in the Qβ-S100A9 group remained alive and continued to be healthy until the last measured timepoint at day 60 indicating a survival improvement of 300% over controls. Representative images at different time points can be seen in FIG. 14C. The survival curves and luminescent measurements indicate that the Qβ-S100A9 vaccine extends and improves survival in mice following surgical removal of TNBC due to the elimination of metastatic disease.

In the current experiment, the first injection was administered prior to surgical removal of the tumor. In the clinic, current standard of care neoadjuvant therapy can last from 3 months to several years, which should easily allow for vaccination in the given time frame⁵⁷. The remainder of the treatments would be administered during adjuvant therapy, which ranges from 5-10 years. It is also important to consider beginning vaccination prior to surgical removal of the tumor, as surgery itself can also lead to metastasis⁵⁸, and the vaccine could potentially also reduce surgery-induced metastases. For TNBC, the relapse rate post-surgery is as high as 29% highlighting the need for strategies to protect patients from metastasis as was achieved by the S100A9 vaccine⁵⁹.

### S100A8/9 Levels are Reduced in Lungs and Sera Following Vaccination.

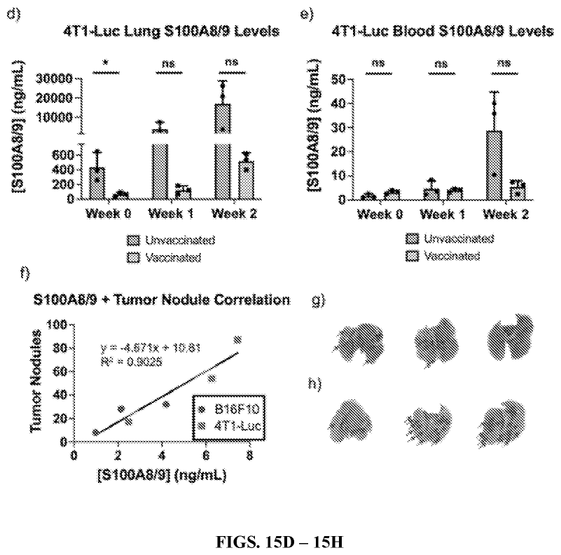

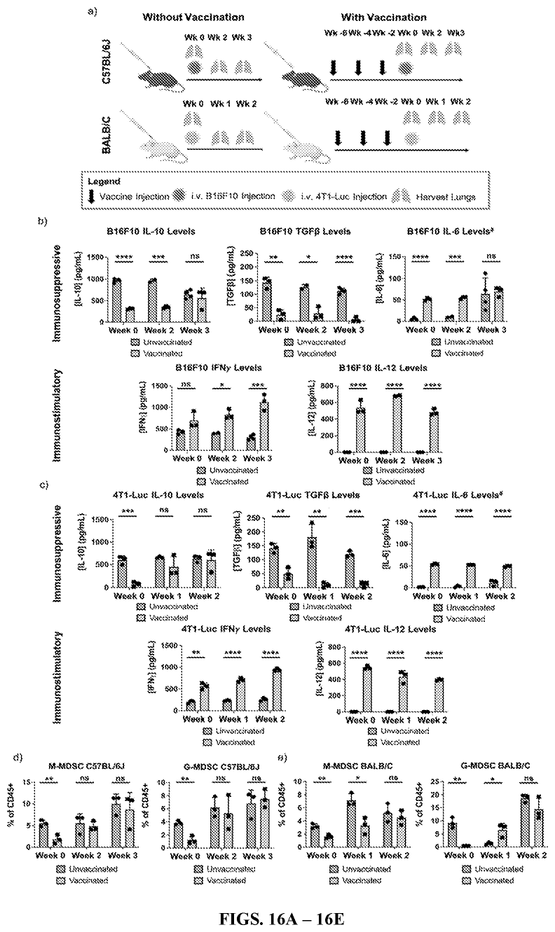

S100A8/9 levels in both the lungs and the sera were measured by ELISA comparing vaccinated and unvaccinated, naïve mice. Applicant used S100A8/9, because S100A9 is typically found in the heterodimer form⁹. Naïve or vaccinated C57BL/6J mice were injected i.v. using 50,000 B16F10 cells, then lungs and sera were collected pre-tumor challenge and 2 and 3 weeks post-tumor challenge (FIG. 15A, top left).

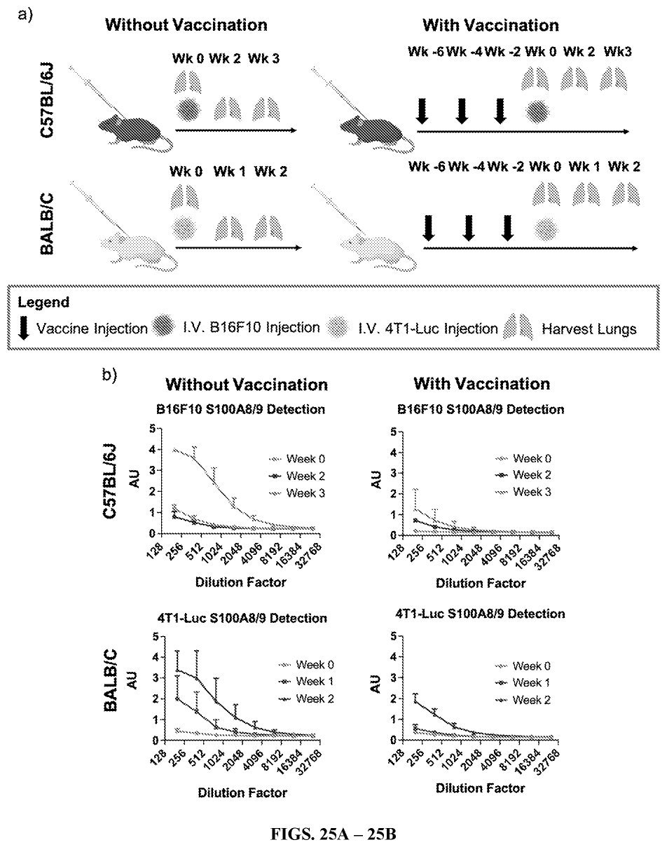

In the naïve mice, the S100A8/9 levels within the lungs remained similar before tumor challenge and 2 weeks post-tumor challenge (1504 vs. 664 ng mL⁻¹); however, by week 3, there was a sudden 10× increase to 13,767 ng mL-1 (FIG. 15B). In stark contrast, in vaccinated mice, the S100A8/9 levels decreased in the lungs to 66.4 ng mL-1 prior to tumor injection, and even 2 and 3 weeks following injection, the levels remained much lower at 182.2 and 360.3 ng mL-1, respectively (FIG. 15B). Strikingly, the S100A8/9 levels within the lungs of vaccinated mice 3 weeks post tumor-injection were still lower than naïve, unvaccinated mice showcasing the longitudinal ability of the vaccine to continuously lower S100A8/9 levels. The full ELISAs are shown in FIG. 25B. Applicant did not observe complete eradication of S100A8/9 within the lungs following vaccination, which may be a positive result given the central role of S100A8/9 in pathogenic clearance^{60, 61}. While detailed safety studies must be carried out to pave the way for translational development of this cancer vaccine candidate, Applicant notes that no apparent adverse effects were observed in the instant study. This is in line with other works in which S100A9 was targeted by means of siRNA or small molecule inhibitors^{23, 62}. It is also important to note that impaired immunity or increased infection rates were not noted as adverse effects in clinical trials of tasquinimod, which is a small molecule inhibitor of S100A9⁶³.

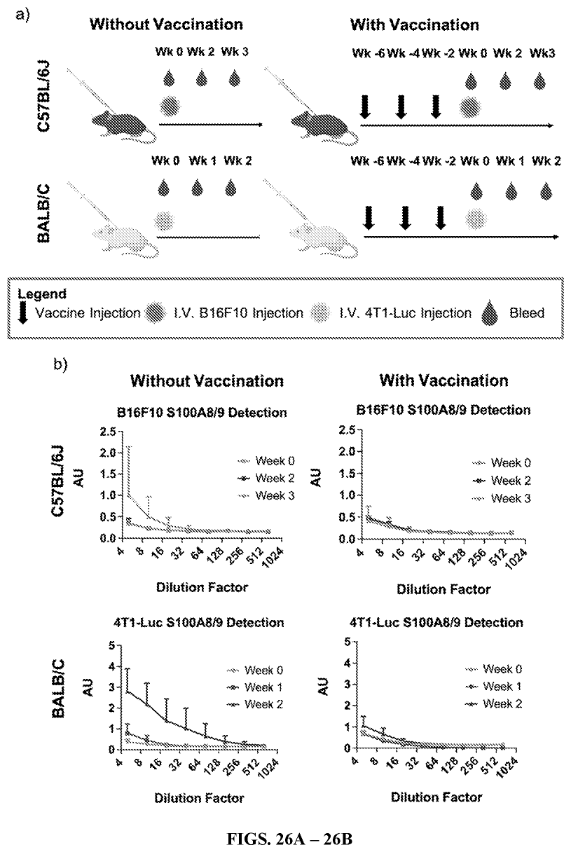

To ascertain that the decrease in the S100A8/9 in the lungs is not attributed to a decrease in tumor nodules, but more so the activity of the α-S100A9 antibodies, Applicant also examined S100A8/9 levels in the sera. While systemic S100A8/9 levels fluctuate depending on disease state, S100A8/9 is mainly active locally, and decreases in systemic S100A8/9 should be mediated by vaccine-generated antibodies. By week 3 following B16F10 i.v. challenge, vaccinated groups showed trends towards decreased S100A8/9 sera levels compared to control animals (FIG. 15C). The 4T1-Luc data showed similar response to the vaccines with significant decreases in the lungs and trends in the sera of S100A8/9 (FIG. 15D, FIG. 15E). The complete ELISAs are shown in FIG. 26B.

The lungs of both 4T1-Luc and B16F10-injected, vaccinated mice at the last measured time point (week 3 for B16F10, week 2 for 4T1-Luc) were then analyzed to investigate correlation between S100A8/9 sera levels and tumor nodule formation (FIGS. 15F-15H). The sera of the mice were collected at the noted timepoints, and S100A8/9 levels were measured through ELISA and correlated to the number of tumor nodules. Indeed, mice with more tumor nodules also had elevated levels of S100A8/9. A line of best fit was calculated, which produced an R2 of 0.9025. The correlation between S100A8/9 and tumor burden further validates that vaccination eliminates S100A8/9 leading to reduction of lung tumor nodules.

### Cytokine and Immune Cell Analysis of Lungs Following Vaccination.

S100A9 plays a direct immunomodulatory role on MDSCs by potentiating their immunosuppressive effects through both paracrine and autocrine functions thereby promoting tumor growth²². To gain insights into the mechanism of action of the Qβ-S100A9 vaccine candidate, Applicant performed cytokine analysis using ELISAs on homogenized lungs (FIG. 16A). Applicant focused on IL-10, IL-6, TGFβ, IFNγ, and IL-12, all of which are directly or indirectly related to MDSC function⁶⁴⁻⁶⁸. Overall, for the B16F10 mice, Applicant noticed sharp decreases in immunosuppressive cytokines and an increase in immunostimulatory cytokines in Qβ-S100A9-vaccinated mice compared to unvaccinated mice (FIG. 16B). Before tumor injection, immunosuppressive cytokines IL-10 and TGFβ were decreased by 3.1- and 5.9-fold in vaccinated mice. These significant decreases continued into week 2 for IL-10 and into week 3 for TGFβ. The cytokine IL-6 was significantly increased 8.6-fold in vaccinated mice at week 0 (FIG. 16B). While IL-6 plays a role in upregulation of MDSCs⁶⁴, it also has known antitumoral effects via simulating adaptive immunity and maturation of APCs^{69, 70}. Importantly, the immunostimulatory cytokines of IFNγ and IL-12 were strongly upregulated in vaccinated mice. By the third week, IFNγ levels were 3.7-fold that of unvaccinated mice. IL-12 was not detectable by ELISA in unvaccinated mice, but was present in vaccinated mice at all timepoints. IFNγ and IL-12 both play crucial roles in creating an immunostimulatory, or “hot”, TME (71-75). IL-12 directly regulates the antitumoral properties of natural killer cells and T cells, which go on to release IFNγ^{71-73, 76}. The IFNγ further activates Th1 effector mechanisms, which produces more IFNγ in a positive feedback loop. Downstream effects are increased differentiation of CD8+ T cells into effector cytotoxic T cells leading to increased tumor cell killing and stimulation of systemic antitumoral immune memory^{74, 75, 77}. Overall, the trends in the 4T1-Luc mouse model were directly comparable with the B16F10 data in that the immunosuppressive cytokines IL-6 and TGFβ were decreased while IL-6, IFNγ, and IL-12 were increased significantly (FIG. 16C). The cytokine analysis indicates that following vaccination of S100A9, there is an overall shift towards a more antitumor, immunostimulatory state, which most likely aids in the rejection of tumor nodule formation.

Applicant also investigated the MDSC populations within the lungs as a function of vaccination against S100A9 (FIG. 16D, FIG. 16E) 78. In C57BL/6J mice, the population of monocytic MDSCs (M-MDSC) decreased by 3-fold while the population of granulocytic MDSCs (G-MDSCs) was decreased 3.1-fold (FIG. 16D) at week 0. Similar trends were observed at week 0 in the 4T1-Luc model using BALB/C (FIG. 16E)-initially, there were significantly lower levels of G-MDSC and M-MDSC populations. Applicant hypothesizes that suppression of G-MDSCs and M-MDSCs in vaccinated mice helps to eliminate tumors at early onset, before the tumors become difficult to eradicate. This is evidenced by the slower tumor growth in the 4T1-Luc injected mice as seen through luminescent imaging (FIG. 13G). Additionally, the main source of S100A9 is attributed to G-MDSCs/neutrophils, and the sharp initial decline in G-MDSCs most likely contributes heavily to the decreased tumor seeding (FIG. 13) and metastasis (FIG. 14). Others have shown that G-MDSCs increase lung metastasis at a higher rate than M-MDSCs, and that depleting G-MDSCs with an anti-Ly6G antibody prevents pulmonary metastasis of i.v.-injected EMT6 cells⁷⁹.

Together, data indicate two distinct mechanisms of action of the Qβ-S100A9 vaccine: 1) the reduction of S100A8/9 decreases tumor seeding into the lungs, and 2) once seeded within the lungs, the tumors develop at slower rates due to the generation of a more antitumor, immunostimulatory environment. Prior research indicates that S100A8/9 recruits cancer cells into the lungs by increasing the expression of cytokines such as TGFβ²⁶. Therefore, reducing S100A8/9 and TGFβ levels synergistically blocks lung metastasis (FIG. 15B, FIG. 15D and FIG. 16B, FIG. 16C). This data are consistent with a report showing that B16F10 cells home to the lungs of uteroglobin knockout (KO) mice which highly express S100A9 within the lungs⁸⁰. Blocking the interaction of S100A9 with B16F10 using antibodies led to decreased metastasis and invasion of the B16F10 cells. An advantage of this vaccine (active immunotherapy) over antibody treatment (passive immunotherapy) is that such therapies will require continuous intervention. By creating a vaccine, the patient will be able to synthesize their own antibodies against S100A9 and continuous drugging may not be necessary, therefore improving quality of life and reducing costs. This additionally pertains to small molecule inhibitors such as tasquinimod, which need to be dosed daily for up to two weeks^{81, 82}.

Another facet of S100A9 is that it promotes MDSC accumulation in both naïve and tumor-bearing mice^{22, 23} By vaccinating against S100A9, the initial M-MDSC and G-MDSC populations are decreased, (FIG. 16D, FIG. 16E) most likely decreasing tumor seeding and growth. In S100A9 KO mice, 9 of 12 implanted tumors were rejected in a lymphoma model due to decreased MDSC populations while the tumors grew aggressively in WT mice²³. Similar reports have been shown in numerous other cancer models^{21, 22, 80, 83-85}. MDSCs are also potent producers of IL-10 and TGFβ71, and Applicant's data clearly indicates that along with diminished MDSC populations, the concentration of immunosuppressive cytokines are decreased following vaccination (FIG. 16B, FIG. 16C). In the TME, IL-10 and TGFβ act on dendritic cells and M1 macrophages restricting their production of IL-12, which acts on natural killer cells to promote antitumor immunity⁷¹⁻⁷³. The downstream effects are that IFNγ production and thereby CD8+ T cell activity and tumor cell killing are decreased^{74, 75}. Indeed, in both the B16F10 and 4T1-Luc models, IL-10 and TGFβ are significantly upregulated in unvaccinated mice while IFNγ and IL-12 become downregulated (FIG. 16B, FIG. 16C)-vaccination helps to reverse this effect. The flow cytometry data coupled with the cytokine analysis helps to validate that vaccination works to negate the effects of the MDSC populations within the lungs thereby reducing metastasis/tumor seeding and growth of the injected tumors.

Without being bound by theory, the disclosed vaccines can be expanded to prevent metastatic outgrowths in organs outside of the lungs and in other cancer types outside of melanoma and TNBC.

### Conclusion

This data shows that vaccination against S100A9 utilizing CPMV and Qβ as an adjuvant and display platform can prime the immune system to reduce S100A9 levels pre- and post-tumor challenge. This leads to reprogramming of the pre-metastatic niche and TME and protection from tumor challenge of melanoma and TNBC. Immunization using Qβ-S100A9 and CPMV-S100A9 induced high titers of α-S100A9 antibodies with Qβ-S100A9 generating higher titers than CPMV-S100A9. Safety of the vaccine candidates is indicated as cross-reactivity to S100A8 was not apparent. Potent efficacy was demonstrated in mouse models of melanoma and TNBC with dramatic reduction of tumor nodules and delayed onset of tumor growth. A clinically relevant primary tumor surgery model also showcased improved trends in survival in vaccinated mice. Mechanism studies revealed reduced S100A8/9 levels in the lungs and sera, changes in cytokine profiles skewing towards an antitumor, immunostimulatory TME along with suppression of MDSCs. In summary, the CPMV and Qβ-S100A9 vaccines demonstrate significant efficacy in reducing the onset of metastatic outgrowths to the lungs of melanoma and TNBC and warrants further investigation into its use as a vaccine platform against metastatic cancers.

### Materials and Methods

### Preparation of S100A9-Subunit Vaccines.

CPMV nanoparticles were propagated in black eyed pea No. 5 plants and purified as previously reported⁴³. Qβ VLPs were expressed in B121 (DE3) (New England BioLabs), and purified as previously reported³⁸.

CPMV and Qβ were first conjugated to a hetero-bifunctional linker, SM(PEG)₈, for 2 hours followed by ultracentrifugation at 52,000 g for 1 h at 4° C. The S100A9 peptide (sequence: CGSGRGHGHSHGKG) (SEQ ID NO: 8) was reacted with the viruses for 2 h at RT and then purified using a 12-14 kDa MWCO dialysis membrane (Avantor) in 10 mM KP.

### Characterization of CPMV-S100A9 and Qβ-S100A9 Particles.

The CPMV and Qβ-S100A9 particles were characterized by UV-VIS, SDS-PAGE, agarose gel electrophoresis, TEM, SEC, and DLS as done previously⁹⁰.

### Animal Immunization.

C57BL/6J mice were injected 3× subcutaneously (s.c.) spaced 2 weeks apart with 200 μg of CPMV, CPMV-S100A9, Qβ, Qβ-S100A9, PBS, and the S100A9 peptide. Two weeks after the last dose, the mice were injected intravenously (i.v.) with either 50,000 or 100,000 B16F10 melanoma cells. The sera from the mice were collected every two weeks from week 2 to week 8.

BALB/C mice received the same dosing regimen as with the C57BL/6J mice with the exception that only the Qβ, Qβ-S100A9, and S100A9 peptide groups were tested. At week 6, 50,000 4T1-Luc cells were injected i.v., and sera were collected every two weeks until week 8.

### Antibody Titers Against S100A9 Peptide and VNP/VLPs.

ELISA was performed against the S100A9 peptide using maleimide-activated plates (Thermo Fisher Scientific) according to the manufacturer's instructions. The CPMV and Qβ-S100A9 groups were further analyzed for antibody isotyping against IgGₜₒₜₐₗ, IgG1, IgG2a, IgG2b, IgG2c, IgA, IgM, and IgE.

For antibody titers against the viral carriers, CPMV or Qβ were coated on Microlon 200 plates (Greiner Bio-One) overnight at 4° C. and examined by ELISA like above.

### WB and DBs Against Full-Length S100A8 and S100A9.

WB and DBs against both S100A9 and S100A8 were carried out. SDS-PAGE gels were transferred onto nitrocellulose paper (VWR), and blocked with 10% (w/v) skim milk. The sera at the week 6 timepoint were pooled from the CPMV-S100A9, Qβ-S100A9, and S100A9 peptide only samples and incubated for 1 hr at RT. Following binding with a goat anti-mouse HRP secondary antibody for 1 hr, a 3,3′-diaminobenzidine (DAB) substrate was added for 2 min and washed away. The blots were then imaged on an AlphaImager system. For the DBs, the S100A8 and S100A9 proteins were directly added to the nitrocellulose paper before analysis like the WB.

### Lung Tumor Seeding B16F10 and 4T1-Luc Model.

Naïve and vaccinated mice were challenged i.v. with B16F10 and 4T1-Luc cells as in Section 3. C57BL/6J mice lungs were harvested after 2 or 3 weeks (depending on injection of 50,000 vs 100,000 cells, respectively) and stored in 10% (v/v) neutral-buffered formalin (Sigma-Aldrich) followed by 70% (v/v) EtOH. The tumor nodules were then manually counted. BALB/C mice injected with 4T1-Luc were analyzed via luminescence imaging using an IVIS (Xenogen). The mice were injected intraperitoneally (i.p.) with 150 mg kg-1 and luminescence was measured using ROI measurements. The lungs were collected after 2 weeks and stored in Bouin's solution (Sigma-Aldrich) followed by 70% (v/v) EtOH.

### 4T1-Luc Primary Cancer Surgical Removal Metastasis Study.

BALB/C mice were injected s.c. with either PBS, Qβ, Qβ-S100A9, or S100A9 peptide only (200 μg mouse-1) as described in Section 3. During the first boost, mice were also injected s.c. in the left flank with 200,000 4T1-Luc cells in 100 μL of PBS. The s.c. tumors were surgically removed two weeks PTI and the skin was sutured using Vetbond tissue adhesive (3M). The mice were then subjected to luminescence imaging as in Section 6, and ROI measurements of the lungs were taken to assess lung metastasis between groups.

### S100A8/9 Levels in the Lungs and Serum Following Vaccination.

S100A8/9 levels within the lungs and sera were analyzed using a mouse S100A8/9 detection kit (R&D Systems) according to the manufacturer's instructions. Prior to the ELISAs, the lungs were harvested at weeks 0, 1, and 3 (in C57BL/6J mice) and weeks 0, 1, and 2 (in BALB/C mice) in both vaccinated and naïve mice and homogenized using a LabGEN 125 homogenizer (Cole-Parmer). Analysis of sera was accomplished through sera collected through r.o. bleeding in both vaccinated and naïve mice at the same timepoints.

### Cytokine Analysis of Lungs.

The lungs of both vaccinated and unvaccinated BALB/C and C57BL/6J mice were analyzed for expression of IL-6, IL-10, IL-12, TGFβ, and IFNγ through ELISA (ThermoFisher) according to the manufacturer's instructions. The lungs were collected at the same timepoints as in Section 8, and homogenized and dissociated in tissue extraction reagent II (ThermoFisher) supplemented with a protease inhibitor cocktail (ThermoFisher) and 10 mM PMSF.

### Flow Cytometry of Lungs.

The lungs of both vaccinated and unvaccinated BALB/C and C57BL/6J mice were collected as before and dissociated into single-cell suspensions using a lung dissociation kit (Militenyi Biotec) according to the manufacturer's instructions. The cells were stained with LIVE/DEAD Aqua (Thermo Scientific) and blocked with 1 μg mL-1 of an Fc block solution (Biolegend). The cells were then stained with the following antibodies (Biolegend): Pacific Blue CD45, SuperBright 645-CD11b, PE-eFluor610-Ly6G, and PE/Cy7-Ly6C. Flow cytometry was done using a BD FACSCelesta and data analysis was done using FlowJo.

### Materials

Potassium phosphate monobasic and dibasic anhydrates were purchased from Fisher. Phosphate buffered saline (PBS) was purchased from both Corning and G Biosciences. Sodium phosphate was purchased from Thermo Fisher Scientific, sodium chloride was purchased from Fisher Scientific, and ethylenediaminetetraacetic acid (EDTA) was purchased from Sigma-Aldrich. Tris acetate EDTA (TAE) and morpholinepropanesulfonic (MOPS) acid buffer were both purchased from Thermo Fisher Scientific. Tween-20 was purchased from Thermo Fisher Scientific, and bovine serum albumin (BSA) fraction V was purchased from Millipore Sigma. Pierce 1-Step Ultra 3,3′,5,5′-tetramethylbenzidine (TMB) solution was purchased from Thermo Fisher Scientific. Ethanol (EtOH) was purchased from Sigma-Aldrich while bleach was purchased from Clorox. Chloroform was purchased from Fisher Scientific and 1-butanol was purchased from Sigma-Aldrich. PEG-8000 was purchased from Fisher. Magic Media was purchased from Thermo Fisher Scientific. Maleimide-polyethylene glycol₈-succinimidyl ester (SM(PEG)₈) was purchased from Sigma-Aldrich, and dimethyl sulfoxide was purchased from VWR. The S100A9 peptide (sequence: CGSGRGHGHSHGKG (SEQ ID NO: 8)) was purchased from Genscript.

B16F10 (CRL-6475) and 4T1-Luc (CRL-2539-LUC2) cells were both purchased from ATCC. B16F10 cells were grown in Dulbecco's modified Eagle's medium (DMEM) supplemented with 10% (v/v) fetal bovine serum (FBS) and 1% (v/v) penicillin/streptomycin (P/S). 4T1-Luc cells were grown in Roswell Park Memorial Institute (RPMI)-1640 medium supplemented with 10% (v/v) FBS and 1% (v/v) P/S. DMEM and RPMI-1640 were purchased from Corning while FBS was purchased from R&D Systems. P/S was purchased from Cytiva. The cells were grown at 5% CO₂ and 37° C.

Goat anti-mouse IgG horseradish peroxidase (HRP) secondary antibodies (Invitrogen A16072, RRID AB_2534745) and goat anti-mouse Fc IgG HRP secondary antibodies (Invitrogen A16090, RRID AB_2534764) were purchased from Thermo Fisher Scientific. Goat anti-mouse IgG2a HRP secondary antibodies were purchased from Thermo Scientific (A-10685, RRID AB_2534065) while goat anti-mouse IgG2b (ab97250, clone #unknown) and IgG2c (ab97255, clone #unknown) HRP secondary antibodies were purchased from Abcam. Goat anti-mouse IgE HRP secondary antibodies (Invitrogen PA184764, RRID AB_931454) were purchased from Fisher Scientific. Goat anti-mouse IgM HRP (ab97230, clone #unknown) and goat anti-mouse IgA (ab97235, clone #unknown) HRP secondary antibodies were purchased from Abcam.

### Preparation of S100A9-Subunit Vaccines.

Cowpea mosaic virus (CPMV) nanoparticles were propagated in black eyed pea No. 5 plants and purified as previously reported⁴³. Qβ virus like particles (VLPs) were expressed in B121 (DE3) (New England BioLabs), and purified as previously reported³⁸. CPMV was stored in 0.1 M potassium phosphate buffer (from here on out referred to as KP buffer) (pH 7.2) while Qβ was stored in 1×PBS pH 7.2. Both nanoparticles were stored at 4° C. until further use.

Before conjugation, the buffers of CPMV and Qβ VLPs were exchanged to 10 mM KP buffer using 100 kDa, 0.5 mL molecular weight cut off (MWCO) spin filters (EMD Millipore), as instructed by the manufacturer. CPMV and Qβ VLPs were then modified by adding 10 molar equivalents per CP of the hetero-bifunctional linker, SM(PEG)₈, and the reaction was run for 2 hours at room temperature (RT). For the CPMV, the excess SM(PEG)₈ was removed using ultracentrifugation at 52,000 g for 1 h at 4° C. with a 30% (w/v) sucrose cushion. The SM(PEG)₈ in the Qβ was removed using PD MidiTrap G-25 columns (Cytiva), as instructed by the manufacturer. The final volume of 1.5 mL was reduced down to 500 μL using the same 100 kDa MWCO spin filters from above. The S100A9 peptide (sequence: CGSGRGHGHSHGKG (SEQ ID NO: 8)) was added to both the CPMV-SM(PEG)₈ and the Qβ-SM(PEG)₈ at 1 molar equivalent per CP and allowed to react at RT for 2 hours. The excess peptide from the CPMV solution was filtered out using the same PD MidiTrap G-25 column while excess peptide from the Qβ solution was removed by dialyzing with a 12-14 kDa MWCO dialysis membrane (Avantor) in 10 mM KP. The resulting vaccine candidates CPMV-S100A9 and Qβ-S100A9 were kept at 4° C. until further use.

### Characterization of CPMV-S100A9 and Qβ-S100A9 Particles.

### Concentration

Native CPMV and CPMV-S100A9 particle concentrations were calculated using ultraviolet-visible (UV-VIS) spectroscopy (Nanodrop 2000) and Beer's Law. CPMV has an extinction coefficient of 8.1 mL mg⁻¹ cm⁻¹ at 260 nm; a ratio of 1.8 at A260/280 nm indicates intact and pure CPMV preparations. Qβ and Qβ-S100A9 concentration was carried out using a Pierce™ BCA Assay (Thermo Scientific) according to the manufacturer's protocol.

### Sodium Dodecyl Sulfate—Polyacrylamide Gel Electrophoresis (SDS-PAGE)

CPMV, Qβ, and S100A9-conjugated samples were loaded with 4× lithium dodecyl sulfate Sample Buffer (Life Technologies). In the samples with Qβ, an additional 10× reducing agent (Invitrogen) was added to break disulfide bonds between CPs. The particles were denatured at 95° C. for 5 min, loaded onto a 12% NuPAGE gel (ThermoFisher Scientific), and run at 200 V, 120 mA, and 25 W for 40 min in 1×MOPS buffer. The gels were then visualized using GelCode™ Blue Safe Protein Stain (ThermoFisher Scientific), and imaged on an AlphaImager System (Protein Simple). The number of peptides conjugated to each CP of CPMV and Qβ was calculated using densitometry analysis on ImageJ.

### Agarose Gel Electrophoresis

The samples were stained with 6× Gel Loading Purple dye (Biolabs) before loading onto a 0.8% (w/v) agarose gel stained with 1 μL of GelRed nucleic acid gel stain (Gold Biotechnologies) in 1×TAE buffer. The gels were run for 30 min at 120 V and 400 mA. RNA was imaged under UV light using the AlphaImager System, and the protein bands were imaged using a 0.25% (w/v) Coomassie Blue stain.

### Transmission Electron Microscopy (TEM)

All samples (10 μg protein) were mounted onto Formvar carbon film coated TEM grids (VWR International) and stained with 2% (w/v) uranyl acetate in deionized water prior to imaging using a FEI Tecnai Spirit G2 BioTWIN TEM.

### Size Exclusion Chromatography (SEC)

SEC was performed by fast protein liquid chromatography using an Äkta Pure (Cytiva) with a Superose 6 Increase 10/300 GL column (dimensions: 10×300 mm with exclusion limit of 4×10⁷ Mᵣ). Samples were diluted to 0.1 mg mL⁻¹ in their respective buffers, and absorbance was measured at 260 and 280 nm with an isocratic elution profile.

### Dynamic Light Scattering (DLS)

DLS measurements were carried out on a Zetasizer Nano ZSP/Zen5600 (Malvern Panalytical). The samples were diluted to 0.1 mg mL⁻¹ in 10 mM KP and measured at RT.

### Animal Immunization

All animal experiments were carried out in accordance with the guidelines set out by the IACUC of the University of California, San Diego. All mice were purchased from Jackson Labs and housed at the Moores Cancer Center. The mice were granted unlimited food and water at all times.

C57BL/6J mice were subject to a prime and double-boost vaccine regimen with the injections spaced two weeks apart. The initial groups tested were CPMV, CPMV-S100A9, Qβ, Qβ-S100A9, PBS, and S100A9 peptide only delivered through subcutaneous (s.c.) injection. The viruses were diluted to 1 mg mL⁻¹ in PBS and a total of 200 μL was administered (200 μg per dose); the S100A9 peptide concentration was determined through densitometry analysis of peptide-conjugation efficiency from SDS-PAGE gels of the Qβ-S100A9 vaccine. Two weeks after the last boost (Week 6), the mice were injected intravenously (i.v.) through the tail vein with either 50,000 or 100,000 B16F10 melanoma cells (in 100 μL of PBS) per mouse. The blood of the mice was also collected by subjecting mice to retroorbital (r.o.) bleeding every two weeks starting from the first injection all the way to Week 8. Serum was isolated from the blood by spinning down the blood at 2000× g for 10 min at 4° C. and collecting the clear supernatant. Samples were stored at −80° C. until further use.

BALB/C mice received the same double-boost regiment at the same dose (200 μg) through s.c. injection. In BALB/C mice, only the Qβ, Qβ-S100A9, PBS, and S100A9 peptide groups were tested. Two weeks after the last boost (Week 6), 50,000 4T1-Luc cells that express luciferase were injected i.v. through the tail vein in 100 μL of PBS. Serum was also collected from the mice at two-week intervals until Week 6. Samples were stored at −80° C. until further use.

### Antibody Titers Against S100A9 Peptide.

Sera was analyzed by ELISA against the S100A9 peptide. Maleimide-activated plates (Thermo Fisher Scientific) were utilized for ELISA analysis due to the terminal cysteine in the linker of the S100A9 peptide (sequence: CGSGRGHGHSHGKG (SEQ ID NO: 8)) in accordance with the manufacturer's protocols. Briefly, plates were first washed with 200 μL of PBS+0.1% (v/v) Tween-20 (PBST) three times before coating with 100 μL of 25 μg mL-1 of peptide diluted in 0.1 M sodium phosphate, 0.15 M sodium chloride, 10 mM EDTA, pH 7.2 (binding buffer). The peptide was incubated at 4° C. overnight. Excess peptide was washed away with three 200 μL washes of PBST. The plates were blocked with 100 μL of 10 μg mL⁻¹ L-cysteine (Sigma-Aldrich) in binding buffer for 1 hr at RT followed by 3 washes with PBST. The sera from the mice were diluted in binding buffer at a starting dilution of 1:200 followed by 2-fold dilutions all the way down to a final dilution of 1:25,600. Qβ-S100A9 samples from the C57BL/6J mice started at a dilution of 1:10,000 all the way down to a final dilution of 1:1,280,000. The sera collected from Section 4 were then added to the wells and incubated for 1 hr at RT. Following three PBST wash steps, goat anti-mouse horseradish HRP IgG secondary antibodies specific to the Fc region were diluted in PBST (1:5,000 dilution), and 100 μL of the antibody solution was added and incubated at RT for 1 hr. After one last wash step, 100 μL of 1-Step Ultra TMB was added and incubated at RT for 5 minutes (C57BL/6J samples) or 1 minute (BALB/C samples) before 100 μL addition of 2N H₂SO₄ to quench the reaction. Absorbance was read at 450 nm on a Tecan microplate reader, and endpoint titers were calculated as the dilution at which the absorbance was greater than twice the absorbance of the blank.

### Antibody Isotyping.

The CPMV-S100A9 and Qβ-S100A9 samples were further analyzed by ELISA as before except that at the sera addition step, the sera from five mice at each time point were pooled at a final concentration of 1:1000, which was then run in triplicate. At the secondary antibody step, isotype-specific HRP antibodies (IgGₜₒₜₐₗ, IgG1, IgG2a, IgG2b, IgG2c, IgA, IgM, and IgE) were used instead. All secondary antibodies were used at 1:5000 dilutions except for IgG2a and IgE antibodies, which were diluted 1:1000. The IgG2b IgG1-1 and IgG2c IgG1-1 ratio was calculated for C57BL/6J mice, and a ratio<1 was considered to be a Th2 response. For BALB/C mice, the IgG2b IgG1⁻¹ and IgG2a IgG-1 ratio was utilized.

### Antibody Titers Against VNP/VLPs.

The protocol for testing antibody titers against CPMV and Qβ was modified slightly from Section 5. During the coating step, 20 μg mL-1 of CPMV or Qβ in PBS was used to coat Microlon 200 plates (Greiner Bio-One) before overnight incubation at 4° C. Blocking was carried out by adding 100 μL of 3% (w/v) BSA in PBS for 1 hr at RT. Sera and secondary antibody addition steps remained unchanged; however, TMB was reacted with the HRP secondary antibody for 8 min before quenching the reaction with 2N H₂SO₄. CPMV titers were only tested in C57BL/6J mice while Qβ titers were examined in both C57BL/6J and BALB/C mice.

### Western Blot (WB) and Dot Blot (DB) Against Full-Length S100A8 and S100A9.

WB and DB assays were used to test the ability of the antibodies in the mice sera in binding full-length S100A9 without cross-reacting with S100A8. First, for the WBs, 10 μg of full-length S100A8 and S100A9 proteins (Sino Biological) were run through SDS-PAGE like before, and the gels were transferred over to nitrocellulose paper (VWR) for 1 hr at 25 V, 160 mA, and 17 W. The nitrocellulose was blocked with 10% (w/v) skim milk (Research Product International) diluted in PBS for 1 hr at RT. The paper was then washed 3 times with PBS with 5 min soaks between washes. The sera from five mice at the Week 6 timepoint of the CPMV-S100A9, Qβ-S100A9, and S100A9 peptide only samples were pooled and diluted 1:100 in PBS and incubated with the nitrocellulose paper for 1 hr at RT. The paper was then subject to the same wash steps and incubated with a goat anti-mouse HRP secondary antibody (1:5000 dilution in PBS) for 1 hr at RT. Following washing, a 3,3′-diaminobenzidine (DAB) substrate (Vector Laboratories) was added for 2 min, washed away with PBS, and then imaged on the AlphaImager system.

For the DBs, 4.5 μg of the full-length S100A8 or S100A9 protein was directly added onto the nitrocellulose paper. The paper was blocked with 10% (w/v) skim milk in PBS for 30 min at RT and washed 3 times with PBS. The mice sera from each of the CPMV, Qβ, CPMV-S100A9, Qβ-S100A9, PBS, and S100A9 peptide only groups were pooled separately and diluted 1:100 in PBS before incubation onto the nitrocellulose paper overnight at 4° C. The paper was washed 3 times with PBS, and a goat anti-mouse HRP secondary antibody (1:5000 dilution in PBS) was incubated for 1 hr at RT. After another round of washing, the DAB substrate was added for 2 min before the reaction was stopped by washing away the substrate with PBS and imaging on the AlphaImager system.

### Lung Tumor Seeding B16F10 and 4T1-Luc Model.

Naïve and vaccinated mice were challenged by i.v. injection using B16F10 melanoma cells (50,000 or 100,000 cells) or 4T1-Luc TNBC cells (50,000 cells) as described in Section 4. In the animals injected with 50,000 B16F10 cells, the lungs were harvested after three weeks and moved into 10 mL of 10% (v/v) neutral-buffered formalin solution (Sigma-Aldrich). The lungs of animals injected with 100,000 B16F10 cells were collected after two weeks. The next day, the lungs were moved from the formalin into 70% (v/v) EtOH and tumor nodules were manually counted.

The mice that were injected with 4T1-Luc were analyzed via luminescence imaging. The mice were injected intraperitoneally (i.p.) with 150 mg kg-1 of D-luciferin (Gold Biotechnologies) and imaged using the in vivo imaging system (IVIS) (Xenogen) every two days starting four days post tumor injection (PTI). The luminescence of the lungs was measured through region of interest (ROI) measurements using the Living Image 3.0 software, and the weight of the mice was also measured during imaging. Two weeks PTI, the lungs were harvested and stored in 10 mL of Bouin's solution (Sigma-Aldrich) overnight and moved to 70% EtOH the next day. The tumor nodules were then manually counted.

### 4T1-Luc Primary Cancer Surgical Removal Metastasis Study.

BALB/C mice were injected s.c. with either PBS, Qβ, Qβ-S100A9, or S100A9 peptide only (200 μg mouse-1) in a prime and double-boost vaccine regimen as described in Section 4. During the first boost, mice were also injected s.c. in the left flank with 200,000 4T1-Luc cells in 100 μL of PBS. The s.c. tumors were surgically removed two weeks PTI and the skin was sutured using Vetbond tissue adhesive (3M). Following surgery, mice were given the anesthetic lidocaine (Vet One). The mice were then subjected to luminescence imaging as in Section 8, and ROI measurements of the lungs were taken to assess lung metastasis between groups.

### S100A8/9 Levels in the Lungs and Serum Following Vaccination.

A mouse S100A8/9 detection kit (R&D Systems) was used to determine the levels of S100A8/9 in the lungs and sera; vaccinated and unvaccinated groups were studied pre- and post-tumor challenge using 50,000 B16F10 or 4T1-Luc cells (i.v. in 100 μL) in C57BL/6J or BALB/C mice. The lungs of the C57BL/6J mice were harvested at weeks 0, 2, and 3 post-tumor challenge; the lungs of the BALB/C mice were harvested at weeks 0, 1 and 2 and stored at −20° C. until further use. The lungs were thawed and then homogenized with a LabGEN 125 homogenizer (Cole-Parmer) in 1 mL of PBS. The homogenate was centrifuged at 10,000 g for 10 min, and the supernatants were stored at −80° C. until S100A8/9 detection by ELISA as instructed by the manufacturer (R&D Systems). In brief, Maxisorp plates (Thermo Scientific) were coated with 100 μL of the capture antibody at 4 μg mL-1 and incubated at 4° C. overnight on a platform shaker. The next day, the plates were washed 3 times with PBST and blocked with 1% (w/v) BSA in PBS for one hour at RT. After a wash step, the lung homogenates were diluted 1:200 in PBST and serially diluted to a final dilution of 1:25600 before incubation for 1.5 h at RT. The plates were washed, and 100 μL of a 40 ng mL-1 detection antibody solution was incubated for 1.5 h at RT. Following washing, 100 μL of streptavidin-HRP (diluted 40× from the stock) was incubated for 20 min at RT. The excess streptavidin-HRP was washed away and 100 μL of TMB was added for 20 min followed by 100 μL addition of 2N H₂SO₄. The plates were then read at 450 nm on a Tecan microplate reader.

Sera was collected from both vaccinated and unvaccinated mice at the same timepoints as with the lung S100A8/9 detection, and the sera was isolated and investigated for S100A8/9 levels using ELISA according to the manufacturer's protocols (R&D Systems). The lungs of the mice were also collected, and the tumor burden within the lungs at the last timepoint (week 2 for 4T1-Luc, week 3 for B16F10) were compared to correlate tumor burden with S100A8/9 sera levels.

### Cytokine Analysis of Lungs.

The lungs of vaccinated and unvaccinated BALB/C and C57BL/6J mice were harvested and analyzed for expression of IL-6, IL-10, IL-12, TGFß, and IFNγ through ELISA according to the manufacturer's protocols (ThermoFisher). In unvaccinated mice, BALB/C and C57BL/6J mice were injected i.v. with 50,000 4T1-Luc and B16F10 cells in 200 μL of PBS, respectively. The lungs of mice injected with 4T1-Luc were harvested 1 and 2 weeks post tumor inoculation, and the lungs of B16F10-inoculated mice were harvested 2 and 3 weeks post tumor inoculation. In vaccinated mice, the same injection and lung harvesting schedule was followed. Following lung harvesting, the lungs were dipped immediately into liquid nitrogen and stored at −80° C. until further use. The lungs were then individually weighed, and 10 mL per gram of tissue extraction reagent II (ThermoFisher) supplemented with a protease inhibitor cocktail (ThermoFisher) and 10 mM PMSF was added. The lungs were homogenized, and incubated in the tissue extraction buffer for 2 h at 4° C. followed by centrifugation at 10,000×g at 4° C. The supernatant was collected and analyzed by ELISA according to the manufacturer's protocols.

### Flow Cytometry of Lungs.

The lungs of unvaccinated and vaccinated BALB/C and C57BL/6J mice were analyzed using flow cytometry at the same timepoints as with the cytokine analysis. At each timepoint, the lungs were harvested and immediately digested using a lung dissociation kit (Miltenyi Biotec) according to the manufacturer's protocol. In brief, each lung was added to 2.4 mL of 1× buffer S, 100 μL of enzyme D, and 100 μL of enzyme A in a gentleMACS C tube (Militenyi Biotec). The lungs were digested using the 37C_m_LDK_1 protocol with a gentleMACS dissociator and then centrifuged at 500×g for 5 min at 4° C. The pellet was resuspended in 2 mL of RPMI and then strained over a 70 μm cell strainer. The solution was centrifuged again at 300× g for 10 min at 4° C., and red blood cells were lysed with 1× red blood cell lysis buffer (eBioscience) for 5 min. Following centrifugation, the cells were counted and diluted to 1×10⁷ cells mL⁻¹ in 100 μL of PBS.

The isolated cells were then added to a 96-well V-shape bottom plate and spun down at 500× g for 5 min at 4° C. The supernatant was removed, and the cells were stained with LIVE/DEAD Aqua (Thermo Scientific) diluted 1:1000 in PBS for 20 min at RT. The cells were washed once with 100 μL of FACS buffer (48.15 mL of 1×PBS+100 μL of 0.5M EDTA+500 μL of FBS+1.25 mL of 1M HEPES) and blocked in 1 μg mL⁻¹ of Fc block (Biolegend) solution (101301, [93]) in FACS buffer for 20 min at 4° C. The cells were washed twice, and stained with the following antibodies (all purchased from Biolegend except for the Ly6G antibody, which was purchased at ThermoFisher) at a 1:500 dilution for 1 hr at RT: Pacific Blue CD45 (103125, [30-F11]), SuperBright 645-CD11b (101207, [M1/70]), PE-eFluor610-Ly6G (61-9668-82, [1A8-Ly6G]), and PE/Cy7-Ly6C (128017, [HK1.4]). The cells were washed twice with FACS buffer and fixed with 1× BD fixative solution diluted in deionized water for 10 min at RT. The cells were washed twice with FACS buffer and then kept and stored in FACS buffer at 4° C. until further use. Flow cytometry was done using a BD FACSCelesta, and data analysis was done using FlowJo.

### Statistical Analysis.

All analyses were done on GraphPad Prism. The tumor nodule counts were compared using one-way ANOVA. S100A8/9 levels, cytokine levels, and MDSC populations were analyzed using Student's T-test.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims. All references are herein incorporated in their entirety for any and all purposes.

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES FOR EXPERIMENT NO. 1

1. World Health Organization. www.who.int/news-room/factsheets/detail/cardiovascular-diseases-(cvds) (accessed: Nov. 1, 2021).
2. Gister\dot{a}, A.; Hansson, G. K. The Immunology of Atherosclerosis. Nat Rev Nephrol 2017, 13 (6), 368-380.
3. Robinson, J. G.; Heistad, D. D.; Fox, K. A. A. Atherosclerosis Stabilization with PCSK-9 Inhibition: An Evolving Concept for Cardiovascular Prevention. Atherosclerosis 2015, 243 (2), 593-597.
4. Ridker, P. M.; Everett, B. M.; Thuren, T.; MacFadyen, J. G.; Chang, W. H.; Ballantyne, C.; Fonseca, F.; Nicolau, J.; Koenig, W.; Anker, S. D.; Kastelein, J. J. P.; Cornel, J. H.; Pais, P.; Pella, D.; Genest, J.; Cifkova, R.; Lorenzatti, A.; Forster, T.; Kobalava, Z.; Vida-Simiti, L.; Flather, M.; Shimokawa, H.; Ogawa, H.; Dellborg, M.; Rossi, P. R. F.; Troquay, R. P. T.; Libby, P.; Glynn, R. J. Antiinflammatory Therapy with Canakinumab for Atherosclerotic Disease. New England Journal of Medicine 2017, 377 (12), 1119-1131.
5. Boekholdt, S. M.; Hovingh, G. K.; Mora, S.; Arsenault, B. J.; Amarenco, P.; Pedersen, T. R.; LaRosa, J. C.; Waters, D. D.; DeMicco, D. A.; Simes, R. J.; Keech, A. C.; Colquhoun, D.; Hitman, G. A.; Betteridge, D. J.; Clearfield, M. B.; Downs, J. R.; Colhoun, H. M.; Gotto, A. M.; Ridker, P. M.; Grundy, S. M.; Kastelein, J. J. P. Very Low Levels of Atherogenic Lipoproteins and the Risk for Cardiovascular Events: A Meta-Analysis of Statin Trials. Journal of the American College of Cardiology 2014, 64 (5), 485-494.
6. Tousoulis, D.; Oikonomou, E.; Economou, E. K.; Crea, F.; Kaski, J. C. Inflammatory Cytokines in Atherosclerosis: Current Therapeutic Approaches. Eur Heart J 2016, 37 (22), 1723-1732.
7. Tedgui, A.; Mallat, Z. Cytokines in Atherosclerosis: Pathogenic and Regulatory Pathways. Physiol Rev 2006, 86 (2), 515-581.
8. Wang, S.; Song, R.; Wang, Z.; Jing, Z.; Wang, S.; Ma, J. S100A8/A9 in Inflammation. Frontiers in Immunology 2018, 9.
9. Hessian, P. A.; Edgeworth, J.; Hogg, N. MRP-8 and MRP-14, Two Abundant Ca(2+)-Binding Proteins of Neutrophils and Monocytes. J Leukoc Biol 1993, 53 (2), 197-204.
10. Vogl, T.; Tenbrock, K.; Ludwig, S.; Leukert, N.; Ehrhardt, C.; van Zoelen, M. A. D.; Nacken, W.; Foell, D.; van der Poll, T.; Sorg, C.; Roth, J. Mrp8 and Mrp14 Are Endogenous Activators of Toll-like Receptor 4, Promoting Lethal, Endotoxin-Induced Shock. Nat Med 2007, 13 (9), 1042-1049.
11. Boyd, J. H.; Kan, B.; Roberts, H.; Wang, Y.; Walley, K. R. S100A8 and S100A9 Mediate Endotoxin-Induced Cardiomyocyte Dysfunction via the Receptor for Advanced Glycation End Products. Circ Res 2008, 102 (10), 1239-1246.
12. Hibino, T.; Sakaguchi, M.; Miyamoto, S.; Yamamoto, M.; Motoyama, A.; Hosoi, J.; Shimokata, T.; Ito, T.; Tsuboi, R.; Huh, N.-H. S100A9 Is a Novel Ligand of EMMPRIN That Promotes Melanoma Metastasis. Cancer Res 2013, 73 (1), 172-183.
13. Altwegg, L. A.; Neidhart, M.; Hersberger, M.; Müller, S.; Eberli, F. R.; Corti, R.; Roffi, M.; Sütsch, G.; Gay, S.; von Eckardstein, A.; Wischnewsky, M. B.; Lüscher, T. F.; Maier, W. Myeloid-Related Protein 8/14 Complex Is Released by Monocytes and Granulocytes at the Site of Coronary Occlusion: A Novel, Early, and Sensitive Marker of Acute Coronary Syndromes. Eur Heart J 2007, 28 (8), 941-948.
14. Healy, A. M.; Pickard, M. D.; Pradhan, A. D.; Wang, Y.; Chen, Z.; Croce, K.; Sakuma, M.; Shi, C.; Zago, A. C.; Garasic, J.; Damokosh, A. I.; Dowie, T. L.; Poisson, L.; Lillie, J.; Libby, P.; Ridker, P. M.; Simon, D. I. Platelet Expression Profiling and Clinical Validation of Myeloid-Related Protein-14 as a Novel Determinant of Cardiovascular Events. Circulation 2006, 113 (19), 2278-2284.
15. Katashima, T.; Naruko, T.; Terasaki, F.; Fujita, M.; Otsuka, K.; Murakami, S.; Sato, A.; Hiroe, M.; Ikura, Y.; Ueda, M.; Ikemoto, M.; Kitaura, Y. Enhanced Expression of the S100A8/A9 Complex in Acute Myocardial Infarction Patients. Circ J 2010, 74 (4), 741-748.
16. Ionita, M. G.; Vink, A.; Dijke, I. E.; Laman, J. D.; Peeters, W.; van der Kraak, P. H.; Moll, F. L.; de Vries, J.-P. P. M.; Pasterkamp, G.; de Kleijn, D. P. V. High Levels of Myeloid-Related Protein 14 in Human Atherosclerotic Plaques Correlate with the Characteristics of Rupture-Prone Lesions. Arterioscler Thromb Vasc Biol 2009, 29 (8), 1220-1227.
17. Kunutsor, S. K.; Flores-Guerrero, J. L.; Kieneker, L. M.; Nilsen, T.; Hidden, C.; Sundrehagen, E.; Seidu, S.; Dullaart, R. P. F.; Bakker, S. J. L. Plasma Calprotectin and Risk of Cardiovascular Disease: Findings from the PREVEND Prospective Cohort Study. Atherosclerosis 2018, 275, 205-213.
18. Croce, K.; Gao, H.; Wang, Y.; Mooroka, T.; Sakuma, M.; Shi, C.; Sukhova, G. K.; Packard, R. R. S.; Hogg, N.; Libby, P.; Simon, D. I. Myeloid-Related Protein-8/14 Is Critical for the Biological Response to Vascular Injury. Circulation 2009, 120 (5), 427-436.
19. Hobbs, J. A. R.; May, R.; Tanousis, K.; McNeill, E.; Mathies, M.; Gebhardt, C.; Henderson, R.; Robinson, M. J.; Hogg, N. Myeloid Cell Function in MRP-14 (S100A9) Null Mice. Mol Cell Biol 2003, 23 (7), 2564-2576.
20. Ridker, P. M.; Everett, B. M.; Thuren, T.; MacFadyen, J. G.; Chang, W. H.; Ballantyne, C.; Fonseca, F.; Nicolau, J.; Koenig, W.; Anker, S. D.; Kastelein, J. J. P.; Cornel, J. H.; Pais, P.; Pella, D.; Genest, J.; Cifkova, R.; Lorenzatti, A.; Forster, T.; Kobalava, Z.; Vida-Simiti, L.; Flather, M.; Shimokawa, H.; Ogawa, H.; Dellborg, M.; Rossi, P. R. F.; Troquay, R. P. T.; Libby, P.; Glynn, R. J. Antiinflammatory Therapy with Canakinumab for Atherosclerotic Disease. New England Journal of Medicine 2017, 377 (12), 1119-1131.
21. Ljung, L.; Rantapää-Dahlqvist, S.; Jacobsson, L. T. H.; Askling, J. Response to Biological Treatment and Subsequent Risk of Coronary Events in Rheumatoid Arthritis. Ann Rheum Dis 2016, 75 (12), 2087-2094.
22. Sabatine, M. S.; Giugliano, R. P.; Wiviott, S. D.; Raal, F. J.; Blom, D. J.; Robinson, J.; Ballantyne, C. M.; Somaratne, R.; Legg, J.; Wasserman, S. M.; Scott, R.; Koren, M. J.; Stein, E. A.; Open-Label Study of Long-Term Evaluation against LDL Cholesterol (OSLER) Investigators. Efficacy and Safety of Evolocumab in Reducing Lipids and Cardiovascular Events. N Engl J Med 2015, 372 (16), 1500-1509.
23. Robinson, J. G.; Farnier, M.; Krempf, M.; Bergeron, J.; Luc, G.; Averna, M.; Stroes, E. S.; Langslet, G.; Raal, F. J.; El Shahawy, M.; Koren, M. J.; Lepor, N. E.; Lorenzato, C.; Pordy, R.; Chaudhari, U.; Kastelein, J. J. P.; ODYSSEY LONG TERM Investigators. Efficacy and Safety of Alirocumab in Reducing Lipids and Cardiovascular Events. N Engl J Med 2015, 372 (16), 1489-1499.
24. Nissen, S. E.; Stroes, E.; Dent-Acosta, R. E.; Rosenson, R. S.; Lehman, S. J.; Sattar, N.; Preiss, D.; Bruckert, E.; Češka, R.; Lepor, N.; Ballantyne, C. M.; Gouni-Berthold, I.; Elliott, M.; Brennan, D. M.; Wasserman, S. M.; Somaratne, R.; Scott, R.; Stein, E. A.; for the GAUSS-3 Investigators. Efficacy and Tolerability of Evolocumab vs Ezetimibe in Patients With Muscle-Related Statin Intolerance: The GAUSS-3 Randomized Clinical Trial. JAMA 2016, 315 (15), 1580-1590.
25. ICER, Institute for Clinical and Economic Review. PCSK9 Inhibitors for Treatment of High Cholesterol: Effectiveness, Value, and ValueBased Price Benchmarks Final Report. https://icer.org/assessment/high cholesterol-2015/(accessed: Nov. 1, 2021).
26. Nilsson, J.; Hansson, G. K. Vaccination Strategies and Immune Modulation of Atherosclerosis. Circ Res 2020, 126 (9), 1281-1296.
27. Ortega-Rivera, O. A.; Pokorski, J. K.; Steinmetz, N. F. A Single-Dose, Implant-Based, Trivalent Virus-like Particle Vaccine against “Cholesterol Checkpoint” Proteins. Advanced Therapeutics 2021, 4 (8), 2100014.
28. Shao, S.; Ortega-Rivera, O. A.; Ray, S.; Pokorski, J. K.; Steinmetz, N. F. A Scalable Manufacturing Approach to Single Dose Vaccination against HPV. Vaccines 2021, 9 (1), 66.
29. Ortega-Rivera, O. A.; Shin, M. D.; Chen, A.; Beiss, V.; Moreno-Gonzalez, M. A.; Lopez-Ramirez, M. A.; Reynoso, M.; Wang, H.; Hurst, B. L.; Wang, J.; Pokorski, J. K.; Steinmetz, N. F. Trivalent Subunit Vaccine Candidates for COVID-19 and Their Delivery Devices. J. Am. Chem. Soc. 2021, 143 (36), 14748-14765.
30. Shin, M. D.; Shukla, S.; Chung, Y. H.; Beiss, V.; Chan, S. K.; Ortega-Rivera, O. A.; Wirth, D. M.; Chen, A.; Sack, M.; Pokorski, J. K.; Steinmetz, N. F. COVID-19 Vaccine Development and a Potential Nanomaterial Path Forward. Nat. Nanotechnol. 2020, 15 (8), 646-655.
31. Bachmann, M. F.; Jennings, G. T. Therapeutic Vaccines for Chronic Diseases: Successes and Technical Challenges. Philos Trans R Soc Lond B Biol Sci 2011, 366 (1579), 2815-2822.
32. Lee, P. W.; Shukla, S.; Wallat, J. D.; Danda, C.; Steinmetz, N. F.; Maia, J.; Pokorski, J. K. Biodegradable Viral Nanoparticle/Polymer Implants Prepared via Melt-Processing. ACS Nano 2017, 11 (9), 8777-8789.
33. Wirth, D. M.; Pokorski, J. K. Design and Fabrication of a Low-Cost Pilot-Scale Melt-Processing System. Polymer 2019, 181, 121802.
34. Ortega-Rivera, O. A.; Shukla, S.; Shin, M. D.; Chen, A.; Beiss, V.; Moreno-Gonzalez, M. A.; Zheng, Y.; Clark, A. E.; Carlin, A. F.; Pokorski, J. K.; Steinmetz, N. F. Cowpea Mosaic Virus Nanoparticle Vaccine Candidates Displaying Peptide Epitopes Can Neutralize the Severe Acute Respiratory Syndrome Coronavirus. ACS Infect. Dis. 2021, 7 (11), 3096-3110.
35. Jespersen, M. C.; Peters, B.; Nielsen, M.; Marcatili, P. BepiPred-2.0: Improving Sequence-Based B-Cell Epitope Prediction Using Conformational Epitopes. Nucleic Acids Res 2017, 45 (W1), W24-W29.
36. Kawano, T.; Shimamura, M.; Nakagami, H.; Iso, T.; Koriyama, H.; Takeda, S.; Baba, K.; Sasaki, T.; Sakaguchi, M.; Morishita, R.; Mochizuki, H. Therapeutic Vaccine Against S100A9 (S100 Calcium-Binding Protein A9) Inhibits Thrombosis Without Increasing the Risk of Bleeding in Ischemic Stroke in Mice. Hypertension 2018, 72 (6), 1355-1364.
37. Pettersen, E. F.; Goddard, T. D.; Huang, C. C.; Couch, G. S.; Greenblatt, D. M.; Meng, E. C.; Ferrin, T. E. UCSF Chimera—a Visualization System for Exploratory Research and Analysis. J Comput Chem 2004, 25 (13), 1605-1612.
38. Corbin, B. D.; Seeley, E. H.; Raab, A.; Feldmann, J.; Miller, M. R.; Torres, V. J.; Anderson, K. L.; Dattilo, B. M.; Dunman, P. M.; Gerads, R.; Caprioli, R. M.; Nacken, W.; Chazin, W. J.; Skaar, E. P. Metal Chelation and Inhibition of Bacterial Growth in Tissue Abscesses. Science 2008, 319 (5865), 962-965.
39. Sievers, F.; Wilm, A.; Dineen, D.; Gibson, T. J.; Karplus, K.; Li, W.; Lopez, R.; McWilliam, H.; Remmert, M.; Söding, J.; Thompson, J. D.; Higgins, D. G. Fast, Scalable Generation of High-quality Protein Multiple Sequence Alignments Using Clustal Omega. Mol Syst Biol 2011, 7 (1), 539.
40. Bachmann, M. F.; Jennings, G. T. Vaccine Delivery: A Matter of Size, Geometry, Kinetics and Molecular Patterns. Nat Rev Immunol 2010, 10 (11), 787-796.
41. Chung, Y. H.; Church, D.; Koellhoffer, E. C.; Osota, E.; Shukla, S.; Rybicki, E. P.; Pokorski, J. K.; Steinmetz, N. F. Integrating Plant Molecular Farming and Materials Research for Next-Generation Vaccines. Nat Rev Mater 2021, 1-17.
42. Finkelman, F. D.; Holmes, J.; Katona, I. M.; Urban, J. F.; Beckmann, M. P.; Park, L. S.; Schooley, K. A.; Coffman, R. L.; Mosmann, T. R.; Paul, W. E. Lymphokine Control of in Vivo Immunoglobulin Isotype Selection. Annu Rev Immunol 1990, 8, 303-333.
43. Rittershaus, C. W.; Miller, D. P.; Thomas, L. J.; Picard, M. D.; Honan, C. M.; Emmett, C. D.; Pettey, C. L.; Adari, H.; Hammond, R. A.; Beattie, D. T.; Callow, A. D.; Marsh, H. C.; Ryan, U. S. Vaccine-Induced Antibodies Inhibit CETP Activity in Vivo and Reduce Aortic Lesions in a Rabbit Model of Atherosclerosis. Arterioscler Thromb Vasc Biol 2000, 20 (9), 2106-2112.
44. Pan, Y.; Zhou, Y.; Wu, H.; Chen, X.; Hu, X.; Zhang, H.; Zhou, Z.; Qiu, Z.; Liao, Y. A Therapeutic Peptide Vaccine Against PCSK9. Sci Rep 2017, 7 (1), 12534.
45. Kawakami, R.; Nozato, Y.; Nakagami, H.; Ikeda, Y.; Shimamura, M.; Yoshida, S.; Sun, J.; Kawano, T.; Takami, Y.; Noma, T.; Rakugi, H.; Minamino, T.; Morishita, R. Development of Vaccine for Dyslipidemia Targeted to a Proprotein Convertase Subtilisin/Kexin Type 9 (PCSK9) Epitope in Mice. PLOS One 2018, 13 (2), e0191895.
46. Schiopu, A.; Cotoi, O. S. S100A8 and S100A9: DAMPs at the Crossroads between Innate Immunity, Traditional Risk Factors, and Cardiovascular Disease. Mediators of Inflammation 2013, 2013, e828354.
47. Shabani, F.; Farasat, A.; Mahdavi, M.; Gheibi, N. Calprotectin (S100A8/S100A9): A Key Protein between Inflammation and Cancer. Inflamm. Res. 2018, 67 (10), 801-812.
48. Akache, B.; Weeratna, R. D.; Deora, A.; Thorn, J. M.; Champion, B.; Merson, J. R.; Davis, H. L.; McCluskie, M. J. Anti-IgE Qb-VLP Conjugate Vaccine Self-Adjuvants through Activation of TLR7. Vaccines (Basel) 2016, 4 (1), 3.
49. Sabbisetti, V. S.; Waikar, S. S.; Antoine, D. J.; Smiles, A.; Wang, C.; Ravisankar, A.; Ito, K.; Sharma, S.; Ramadesikan, S.; Lee, M.; Briskin, R.; De Jager, P. L.; Ngo, T. T.; Radlinski, M.; Dear, J. W.; Park, K. B.; Betensky, R.; Krolewski, A. S.; Bonventre, J. V. Blood Kidney Injury Molecule-1 Is a Biomarker of Acute and Chronic Kidney Injury and Predicts Progression to ESRD in Type I Diabetes. J Am Soc Nephrol 2014, 25 (10), 2177-2186.
50. McGill, M. R. The Past and Present of Serum Aminotransferases and the Future of Liver Injury Biomarkers. EXCLI J 2016, 15, 817-828.
51. Kollmus, H.; Fuchs, H.; Lengger, C.; Haselimashhadi, H.; Bogue, M. A.; Östereicher, M. A.; Horsch, M.; Adler, T.; Aguilar-Pimentel, J. A.; Amarie, O. V.; Becker, L.; Beckers, J.; Calzada-Wack, J.; Garrett, L.; Hans, W.; Holter, S. M.; Klein-Rodewald, T.; Maier, H.; Mayer-Kuckuk, P.; Miller, G.; Moreth, K.; Neff, F.; Rathkolb, B.; Rácz, I.; Rozman, J.; Spielmann, N.; Treise, I.; Busch, D.; Graw, J.; Klopstock, T.; Wolf, E.; Wurst, W.; Yildirim, A. Ö.; Mason, J.; Torres, A.; Balling, R.; Mehaan, T.; Gailus-Durner, V.; Schughart, K.; Hrabě de Angelis, M. A Comprehensive and Comparative Phenotypic Analysis of the Collaborative Founder Strains Identifies New and Known Phenotypes. Mamm Genome 2020, 31 (1), 30-48.
52. Ramírez-Zacarías, J. L.; Castro-Muñozledo, F.; Kuri-Harcuch, W. Quantitation of Adipose Conversion and Triglycerides by Staining Intracytoplasmic Lipids with Oil Red O. Histochemistry 1992, 97 (6), 493-497.
53. Vogl, T.; Stratis, A.; Wixler, V.; Völler, T.; Thurainayagam, S.; Jorch, S. K.; Zenker, S.; Dreiling, A.; Chakraborty, D.; Fröhling, M.; Paruzel, P.; Wehmeyer, C.; Hermann, S.; Papantonopoulou, O.; Geyer, C.; Loser, K.; Schäfers, M.; Ludwig, S.; Stoll, M.; Leanderson, T.; Schultze, J. L.; König, S.; Pap, T.; Roth, J. Autoinhibitory Regulation of S100A8/S100A9 Alarmin Activity Locally Restricts Sterile Inflammation. J Clin Invest 2018, 128 (5), 1852-1866.
54. Vogl, T.; Ludwig, S.; Goebeler, M.; Strey, A.; Thorey, I. S.; Reichelt, R.; Foell, D.; Gerke, V.; Manitz, M. P.; Nacken, W.; Werner, S.; Sorg, C.; Roth, J. MRP8 and MRP14 Control Microtubule Reorganization during Transendothelial Migration of Phagocytes. Blood 2004, 104 (13), 4260-4268.
55. Hirata, A.; Kishida, K.; Nakatsuji, H.; Hiuge-Shimizu, A.; Funahashi, T.; Shimomura, I. High Serum S100A8/A9 Levels and High Cardiovascular Complication Rate in Type 2 Diabetics with Ultrasonographic Low Carotid Plaque Density. Diabetes Research and Clinical Practice 2012, 97 (1), 82-90.
56. Holzinger, D.; Nippe, N.; Vogl, T.; Marketon, K.; Mysore, V.; Weinhage, T.; Dalbeth, N.; Pool, B.; Merriman, T.; Baeten, D.; Ives, A.; Busso, N.; Foell, D.; Bas, S.; Gabay, C.; Roth, J. Myeloid-Related Proteins 8 and 14 Contribute to Monosodium Urate Monohydrate Crystal-Induced Inflammation in Gout. Arthritis Rheumatol 2014, 66 (5), 1327-1339.
57. Nishikawa, Y.; Kajiura, Y.; Lew, J. H.; Kido, J.; Nagata, T.; Naruishi, K. Calprotectin Induces IL-6 and MCP-1 Production via Toll-Like Receptor 4 Signaling in Human Gingival Fibroblasts. Journal of Cellular Physiology 2017, 232 (7), 1862-1871.
58. Galea, J.; Armstrong, J.; Gadsdon, P.; Holden, H.; Francis, S. E.; Holt, C. M. Interleukin-1 Beta in Coronary Arteries of Patients with Ischemic Heart Disease. Arterioscler Thromb Vasc Biol 1996, 16 (8), 1000-1006.
59. Bevilacqua, M. P.; Pober, J. S.; Majeau, G. R.; Cotran, R. S.; Gimbrone, M. A. Interleukin 1 (IL-1) Induces Biosynthesis and Cell Surface Expression of Procoagulant Activity in Human Vascular Endothelial Cells. J Exp Med 1984, 160 (2), 618-623.
60. Bevilacqua, M. P.; Pober, J. S.; Wheeler, M. E.; Cotran, R. S.; Gimbrone, M. A. Interleukin 1 Acts on Cultured Human Vascular Endothelium to Increase the Adhesion of Polymorphonuclear Leukocytes, Monocytes, and Related Leukocyte Cell Lines. J Clin Invest 1985, 76 (5), 2003-2011.
61. Grebe, A.; Hoss, F.; Latz, E. NLRP3 Inflammasome and the IL-1 Pathway in Atherosclerosis. Circulation Research 2018, 122 (12), 1722-1740.
62. Kirii, H.; Niwa, T.; Yamada, Y.; Wada, H.; Saito, K.; Iwakura, Y.; Asano, M.; Moriwaki, H.; Seishima, M. Lack of Interleukin-1ß Decreases the Severity of Atherosclerosis in ApoE-Deficient Mice. Arteriosclerosis, Thrombosis, and Vascular Biology 2003, 23 (4), 656-660.
63. Shimamura, M.; Kaikita, K.; Nakagami, H.; Kawano, T.; Ju, N.; Hayashi, H.; Nakamaru, R.; Yoshida, S.; Sasaki, T.; Mochizuki, H.; Tsujita, K.; Morishita, R. Development of Anti-Thrombotic Vaccine against Human S100A9 in Rhesus Monkey. Sci Rep 2021, 11 (1), 11472.
64. Ometto, F.; Friso, L.; Astorri, D.; Botsios, C.; Raffeiner, B.; Punzi, L.; Doria, A. Calprotectin in Rheumatic Diseases. Exp Biol Med (Maywood) 2017, 242 (8), 859-873.
65. Brown, S. D.; Fiedler, J. D.; Finn, M. G. Assembly of Hybrid Bacteriophage Qbeta Virus-like Particles. Biochemistry 2009, 48 (47), 11155-11157.
66. Sreejit, G.; Flynn, M. C.; Patil, M.; Krishnamurthy, P.; Murphy, A. J.; Nagareddy, P. R. Chapter Six—S100 Family Proteins in Inflammation and Beyond. In Advances in Clinical Chemistry; Makowski, G. S., Ed.; Elsevier, 2020; Vol. 98, pp 173-231.
67. Andrés-Manzano, M. J.; Andrés, V.; Dorado, B. Oil Red O and Hematoxylin and Eosin Staining for Quantification of Atherosclerosis Burden in Mouse Aorta and Aortic Root. In Methods in Mouse Atherosclerosis; Andrés, V., Dorado, B., Eds.; Methods in Molecular Biology; Springer: New York, NY, 2015; pp 85-99.

### REFERENCES FOR EXPERIMENT NO. 2

1. A. Wells, J. Grahovac, S. Wheeler, B. Ma, D. Lauffenburger, Targeting tumor cell motility as a strategy against invasion and metastasis. Trends Pharmacol Sci 34, 283-289 (2013).
2. X. Guan, Cancer metastases: challenges and opportunities. Acta Pharm Sin B 5, 402-418 (2015).
3. O. Peart, Metastatic Breast Cancer. Radiol Technol 88, 519M-539M (2017).
4. L. Enewold, E. Sharon, L. C. Harlan, Metastatic Melanoma: Treatment and Survival in the US after the Introduction of Ipilimumab and Vemurafenib. Oncol Res Treat 40, 174-183 (2017).
5. S. Zhang, Y. Wang, Y. Ding, Y. Wu, [Long term survival results in advanced prostate cancer treated with combined androgen blockade]. Zhonghua Nan Ke Xue 11, 770-771, 774 (2005).
6. A. B. Gardner, et al., Ovarian, uterine, and cervical cancer patients with distant metastases at diagnosis: most common locations and outcomes. Clin Exp Metastasis 37, 107-113 (2020).
7. A. Jamil, A. Kasi, “Lung Metastasis” in StatPearls, (StatPearls Publishing, 2022) (May 24, 2022).
8. G. Srikrishna, S100A8 and S100A9: New Insights into Their Roles in Malignancy. J Innate Immun 4, 31-40 (2011).
9. S. Wang, et al., S100A8/A9 in Inflammation. Front. Immunol. 9 (2018).
10. K. Croce, et al., MRP-8/14 is Critical for the Biological Response to Vascular Injury. Circulation 120, 427-436 (2009).
11. C. Gebhardt, J. Németh, P. Angel, J. Hess, S100A8 and S100A9 in inflammation and cancer. Biochem Pharmacol 72, 1622-1631 (2006).
12. I. Salama, P. S. Malone, F. Mihaimeed, J. L. Jones, A review of the S100 proteins in cancer. Eur J Surg Oncol 34, 357-364 (2008).
13. H. M. Koh, et al., Prognostic role of S100A9 expression in patients with clear cell renal cell carcinoma. Medicine (Baltimore) 98, e17188 (2019).
14. J. Liao, et al., High S100A9⁺ cell density predicts a poor prognosis in hepatocellular carcinoma patients after curative resection. Aging 13, 16367-16380 (2021).
15. J. Meng, F. Gu, H. Fang, B. Qu, Elevated Serum S100A9 Indicated Poor Prognosis in Hepatocellular Carcinoma after Curative Resection. J Cancer 10, 408-415 (2019).
16. Z. Lv, W. Li, X. Wei, S100A9 promotes prostate cancer cell invasion by activating TLR4/NF-κB/integrin β1/FAK signaling. Onco Targets Ther 13, 6443-6452 (2020).
17. R. Song, K. Struhl, S100A8/S100A9 cytokine acts as a transcriptional coactivator during breast cellular transformation. Science Advances 7, eabe5357 (2021).
18. W.-Y. Fang, et al., Elevated S100A9 expression in tumor stroma functions as an early recurrence marker for early-stage oral cancer patients through increased tumor cell invasion, angiogenesis, macrophage recruitment and interleukin-6 production. Oncotarget 6, 28401-28424 (2015).
19. K. Arai, et al., S100A8 and S100A9 overexpression is associated with poor pathological parameters in invasive ductal carcinoma of the breast. Curr Cancer Drug Targets 8, 243-252 (2008).
20. C. Zhao, et al., S100A9 regulates cisplatin chemosensitivity of squamous cervical cancer cells and related mechanism. Cancer Manag Res 10, 3753-3764 (2018).
21. J. Li, et al., S100A9-CXCL12 activation in BRCA1-mutant breast cancer promotes an immunosuppressive microenvironment associated with resistance to immunotherapy. Nat Commun 13, 1481 (2022).
22. P. Sinha, et al., Proinflammatory S100 Proteins Regulate the Accumulation of Myeloid-Derived Suppressor Cells. J Immunol 181, 4666-4675 (2008).
23. P. Cheng, et al., Inhibition of dendritic cell differentiation and accumulation of myeloid-derived suppressor cells in cancer is regulated by S100A9 protein. J Exp Med 205, 2235-2249 (2008).
24. O. Turovskaya, et al., RAGE, carboxylated glycans and S100A8/A9 play essential roles in colitis-associated carcinogenesis. Carcinogenesis 29, 2035-2043 (2008).
25. P. Goedegebuure, et al., Myeloid-derived suppressor cells: general characteristics and relevance to clinical management of pancreatic cancer. Curr Cancer Drug Targets 11, 734-751 (2011).
26. S. Hiratsuka, A. Watanabe, H. Aburatani, Y. Maru, Tumour-mediated upregulation of chemoattractants and recruitment of myeloid cells predetermines lung metastasis. Nat Cell Biol 8, 1369-1375 (2006).
27. University of Pennsylvania, “Phase 1 Study of Tasquinimod Alone and in Combination With Standard Therapy for Relapsed or Refractory Myeloma” (clinicaltrials.gov, 2022) (Nov. 9, 2022).
28. A. Moon, et al., Global gene expression profiling unveils S100A8/A9 as candidate markers in H-ras-mediated human breast epithelial cell invasion. Mol Cancer Res 6, 1544-1553 (2008).
29. H.-Y. Yong, A. Moon, Roles of calcium-binding proteins, S100A8 and S100A9, in invasive phenotype of human gastric cancer cells. Arch Pharm Res 30, 75-81 (2007).
30. M. O. Mohsen, G. Augusto, M. F. Bachmann, The 3Ds in virus-like particle based-vaccines: “Design, Delivery and Dynamics.” Immunol Rev 296, 155-168 (2020).
31. Y. H. Chung, et al., Integrating plant molecular farming and materials research for next-generation vaccines. Nat Rev Mater, 1-17 (2021).
32. G. Spohn, et al., A Virus-Like Particle-Based Vaccine Selectively Targeting Soluble TNF-α Protects from Arthritis without Inducing Reactivation of Latent Tuberculosis. The Journal of Immunology 178, 7450-7457 (2007).
33. M. F. Bachmann, M. R. Dyer, Therapeutic vaccination for chronic diseases: a new class of drugs in sight. Nat Rev Drug Discov 3, 81-88 (2004).
34. C. I. Nkanga, O. A. Ortega-Rivera, M. D. Shin, M. A. Moreno-Gonzalez, N. F. Steinmetz, Injectable Slow-Release Hydrogel Formulation of a Plant Virus-Based COVID-19 Vaccine Candidate. Biomacromolecules 23, 1812-1825 (2022).
35. S. Ray, D. M. Wirth, O. A. Ortega-Rivera, N. F. Steinmetz, J. K. Pokorski, Dissolving Microneedle Delivery of a Prophylactic HPV Vaccine. Biomacromolecules 23, 903-912 (2022).
36. O. A. Ortega-Rivera, et al., Cowpea Mosaic Virus Nanoparticle Vaccine Candidates Displaying Peptide Epitopes Can Neutralize the Severe Acute Respiratory Syndrome Coronavirus. ACS Infect. Dis. 7, 3096-3110 (2021).
37. O. A. Ortega-Rivera, et al., Trivalent Subunit Vaccine Candidates for COVID-19 and Their Delivery Devices. J. Am. Chem. Soc. 143, 14748-14765 (2021).
38. O. A. Ortega-Rivera, J. K. Pokorski, N. F. Steinmetz, A Single-Dose, Implant-Based, Trivalent Virus-like Particle Vaccine against “Cholesterol Checkpoint” Proteins. Advanced Therapeutics 4, 2100014 (2021).
39. P. H. Lizotte, et al., In situ vaccination with cowpea mosaic virus nanoparticles suppresses metastatic cancer. Nat Nanotechnol 11, 295-303 (2016).
40. T. Kawano, et al., Therapeutic Vaccine Against S100A9 (S100 Calcium-Binding Protein A9) Inhibits Thrombosis Without Increasing the Risk of Bleeding in Ischemic Stroke in Mice. Hypertension 72, 1355-1364 (2018).
41. M. C. Jespersen, B. Peters, M. Nielsen, P. Marcatili, BepiPred-2.0: improving sequence-based B-cell epitope prediction using conformational epitopes. Nucleic Acids Res 45, W24-W29 (2017).
42. W. Nacken, C. Sopalla, C. Pröpper, C. Sorg, C. Kerkhoff, Biochemical characterization of the murine S100A9 (MRP14) protein suggests that it is functionally equivalent to its human counterpart despite its low degree of sequence homology. Eur. J. Biochem. 267, 560-565 (2000).
43. A. M. Wen, et al., Viral Nanoparticles for In vivo Tumor Imaging. J Vis Exp (2012) https:/doi.org/10.3791/4352 (Nov. 8, 2019).
44. S. Shukla, et al., The unique potency of Cowpea mosaic virus (CPMV) in situ cancer vaccine. Biomater. Sci. 8, 5489-5503 (2020).
45. D. J. K. Pokorski, M. L. Hovlid, P. M. G. Finn, Cell Targeting with Hybrid Qβ Virus-Like Particles Displaying Epidermal Growth Factor. Chembiochem: a European journal of chemical biology 12, 2441 (2011).
46. A. Shahrivarkevishahi, et al., PhotothermalPhage: A Virus-Based Photothermal Therapeutic Agent. J. Am. Chem. Soc. 143, 16428-16438 (2021).
47. S. Nazeri, S. Zakeri, A. A. Mehrizi, S. Sardari, N. D. Djadid, Measuring of IgG2c isotype instead of IgG2a in immunized C57BL/6 mice with Plasmodium vivax TRAP as a subunit vaccine candidate in order to correct interpretation of Th1 versus Th2 immune response. Exp Parasitol 216, 107944 (2020).
48. C. Mao, V. Beiss, J. Fields, N. F. Steinmetz, S. Fiering, Cowpea mosaic virus stimulates antitumor immunity through recognition by multiple MYD88-dependent toll-like receptors. Biomaterials 275, 120914 (2021).
49. S. Schulte, G. K. Sukhova, P. Libby, Genetically Programmed Biases in Th1 and Th2 Immune Responses Modulate Atherogenesis. Am J Pathol 172, 1500-1508 (2008).
50. J.-W. Lim, et al., Co-delivery of antigens and immunostimulants via a polymersome for improvement of antigen-specific immune response. J. Mater. Chem. B 8, 5620-5626 (2020).
51. Y. H. Chung, B. A. Volckaert, N. F. Steinmetz, Development of a Modular NTA: His Tag Viral Vaccine for Co-delivery of Antigen and Adjuvant. Bioconjugate Chem. 34, 269-278 (2023).
52. D. S. Watson, V. M. Platt, L. Cao, V. J. Venditto, F. C. Szoka, Antibody response to polyhistidine-tagged peptide and protein antigens attached to liposomes via lipid-linked nitrilotriacetic acid in mice. Clin Vaccine Immunol 18, 289-297 (2011).
53. Z. Zhao, O. A. Ortega-Rivera, Y. H. Chung, A. Simms, N. F. Steinmetz, A co-formulated vaccine of irradiated cancer cells and cowpea mosaic virus improves ovarian cancer rejection. J Mater Chem B (2023) https:/doi.org/10.1039/d2tb02355e. 54. C. D. Lemke-Miltner, et al., Antibody opsonization of a TLR9-agonist-containing virus-like particle enhances in situ immunization. J Immunol 204, 1386-1394 (2020).
55. Checkmate Pharmaceuticals, “A Multicenter, Open-label, Phase 2 Study of Intratumoral CMP-001 in Combination With an Intravenous PD-1-Blocking Antibody in Subjects With Selected Types of Advanced or Metastatic Cancer” (clinicaltrials.gov, 2022) (May 24, 2022).
56. Checkmate Pharmaceuticals, “A Multicenter, Phase 2, Open-label Study of Intratumoral CMP-001 in Combination With Intravenous Pembrolizumab in Subjects With Recurrent or Metastatic Head and Neck Squamous Cell Carcinoma” (clinicaltrials.gov, 2022) (May 24, 2022).
57. C. Selli, A. H. Sims, Neoadjuvant Therapy for Breast Cancer as a Model for Translational Research. Breast Cancer (Auckl) 13, 1178223419829072 (2019).
58 S. Tohme, R. L. Simmons, A. Tsung, Surgery for Cancer: A Trigger for Metastases. Cancer Res 77, 1548-1552 (2017).
59. B. G. Haffty, et al., Locoregional relapse and distant metastasis in conservatively managed triple negative early-stage breast cancer. J Clin Oncol 24, 5652-5657 (2006).
60. H. L. Clark, et al., Zinc and Manganese Chelation by Neutrophil S100A8/A9 (Calprotectin) Limits Extracellular Aspergillus fumigatus Hyphal Growth and Corneal Infection. The Journal of Immunology 196, 336-344 (2016).
61. C. F. Urban, et al., Neutrophil extracellular traps contain calprotectin, a cytosolic protein complex involved in host defense against Candida albicans. PLOS Pathog 5, e1000639 (2009).
62. Y. Li, et al., S100a8/a9 Signaling Causes Mitochondrial Dysfunction and Cardiomyocyte Death in Response to Ischemic/Reperfusion Injury. Circulation 140, 751-764 (2019).
63. N. Gupta, O. Al Ustwani, L. Shen, R. Pili, Mechanism of action and clinical activity of tasquinimod in castrate-resistant prostate cancer. Onco Targets Ther 7, 223-234 (2014).
64. R. Weber, et al., IL-6 as a major regulator of MDSC activity and possible target for cancer immunotherapy. Cellular Immunology 359, 104254 (2021).
65. J. F. Santibanez, S. Bjelica, Transforming Growth Factor-Beta1 and Myeloid-Derived Suppressor Cells Interplay in Cancer. The Open Cancer Immunology Journal 06 (2017).
66. D. Jorgovanovic, M. Song, L. Wang, Y. Zhang, Roles of IFN-γ in tumor progression and regression: a review. Biomarker Research 8, 49 (2020).
67. C. E. Steding, et al., The role of interleukin-12 on modulating myeloid-derived suppressor cells, increasing overall survival and reducing metastasis. Immunology 133, 221-238 (2011).
68. K. Hart, K. Byrne, M. Molloy, E. Usherwood, B. Berwin, IL-10 Immunomodulation of Myeloid Cells Regulates a Murine Model of Ovarian Cancer. Frontiers in Immunology 2 (2011).
69. C. Wang, S. N. Fiering, N. F. Steinmetz, Cowpea Mosaic Virus Promotes Anti-Tumor Activity and Immune Memory in a Mouse Ovarian Tumor Model. Adv Ther (Weinh) 2, 1900003 (2019).
70. F. Martinon, A. Mayor, J. Tschopp, The inflammasomes: guardians of the body. Annu Rev Immunol 27, 229-265 (2009).
71. M. M. Yaseen, N. M. Abuharfeil, H. Darmani, A. Daoud, Mechanisms of immune suppression by myeloid-derived suppressor cells: the role of interleukin-10 as a key immunoregulatory cytokine. Open Biol 10, 200111 (2020).
72. C.-E. Hu, J. Gan, R.-D. Zhang, Y.-R. Cheng, G.-J. Huang, Up-regulated myeloid-derived suppressor cell contributes to hepatocellular carcinoma development by impairing dendritic cell function. Scand J Gastroenterol 46, 156-164 (2011).
73. M. A. Y. Alqudah, M. M. M. Yaseen, M. M. S. Yaseen, HIV-1 strategies to overcome the immune system by evading and invading innate immune system. HIV & AIDS Review 15, 1-12 (2016).
74. M. R. Porembka, et al., Pancreatic adenocarcinoma induces bone marrow mobilization of myeloid-derived suppressor cells which promote primary tumor growth. Cancer Immunol Immunother 61, 1373-1385 (2012).
75. G. Li, et al., Vasoactive intestinal peptide induces CD14+HLA−DR−/low myeloid-derived suppressor cells in gastric cancer. Mol Med Rep 12, 760-768 (2015).
76. R. Parihar, J. Dierksheide, Y. Hu, W. E. Carson, IL-12 enhances the natural killer cell cytokine response to Ab-coated tumor cells. J Clin Invest 110, 983-992 (2002).
77. F. Castro, A. P. Cardoso, R. M. Gonçalves, K. Serre, M. J. Oliveira, Interferon-Gamma at the Crossroads of Tumor Immune Surveillance or Evasion. Frontiers in Immunology 9 (2018).
78. V. Bronte, et al., Recommendations for myeloid-derived suppressor cell nomenclature and characterization standards. Nat Commun 7, 12150 (2016).
79. M. Ouzounova, et al., Monocytic and granulocytic myeloid derived suppressor cells differentially regulate spatiotemporal tumour plasticity during metastatic cascade. Nat Commun 8, 14979 (2017).
80. A. Saha, et al., Lack of an endogenous anti-inflammatory protein in mice enhances colonization of B16F10 melanoma cells in the lungs. J Biol Chem 285, 10822-10831 (2010).
81. C. Sternberg, et al., Randomized, Double-Blind, Placebo-Controlled Phase III Study of Tasquinimod in Men With Metastatic Castration-Resistant Prostate Cancer. JCO 34, 2636-2643 (2016).
82. Active Biotech AB, “A Phase 3 Randomized, Double-Blind, Placebo-Controlled Study of Tasquinimod in Men With Metastatic Castrate Resistant Prostate Cancer” (clinicaltrials.gov, 2015) (Nov. 9, 2022).
83. H. Qin, et al., Generation of a novel therapeutic peptide that depletes MDSC in tumor-bearing mice. Nat Med 20, 676-681 (2014).
84. E. McNeill, N. Hogg, S100A9 has a protective role in inflammation-induced skin carcinogenesis. Int J Cancer 135, 798-808 (2014).
85. S. Cheng, et al., Down-regulation of S100A9 inhibits osteosarcoma cell growth through inactivating MAPK and NF-κB signaling pathways. BMC Cancer 16, 253 (2016).
86. L. Duan, et al., HBx-induced S100A9 in NF-κB dependent manner promotes growth and metastasis of hepatocellular carcinoma cells. Cell Death Dis 9, 1-14 (2018).
87. J. Meng, F. Gu, H. Fang, B. Qu, Elevated Serum S100A9 Indicated Poor Prognosis in Hepatocellular Carcinoma after Curative Resection. J Cancer 10, 408-415 (2019).
88. C. Monteiro, et al., Stratification of radiosensitive brain metastases based on an actionable S100A9/RAGE resistance mechanism. Nat Med 28, 752-765 (2022).
89. F. Morandi, et al., Bone Marrow-Infiltrating Human Neuroblastoma Cells Express High Levels of Calprotectin and HLA-G Proteins. PLOS ONE 7, e29922 (2012).
90 Y. H. Chung, J. Park, H. Cai, N. F. Steinmetz, S100A9-Targeted Cowpea Mosaic Virus as a Prophylactic and Therapeutic Immunotherapy against Metastatic Breast Cancer and Melanoma. Advanced Science n/a, 2101796.

### List of Sequences

Protein, Homo sapiens
SEQ ID NO: 1
PGHHHKPGLG,
Protein, Mus musculus
SEQ ID NO: 2
RGHGHSHGKG,
Protein, Cowpea mosaic virus (strain SB)
SEQ ID NO: 3
MEQNLFALSLDDTSSVRGSLLDTKFAQTRVLLSKAMAGGDVLLDEYLYDV
VNGQDFRATVAFLRTHVITGKIKVTATTNISDNSGCCLMLAINSGVRGKY
STDVYTICSQDSMTWNPGCKKNFSFTFNPNPCGDSWSAEMISRSRVRMTV
ICVSGWTLSPTTDVIAKLDWSIVNEKCEPTIYHLADCQNWLPLNRWMGKL
TFPQGVTSEVRRMPLSIGGGAGATQAFLANMPNSWISMWRYFRGELHFEV
TKMSSPYIKATVTFLIAFGNLSDAFGFYESFPHRIVQFAEVEEKCTLVFS
QQEFVTAWSTQVNPRTTLEADGCPYLYAIIHDSTTGTISGDFNLGVKLVG
IKDFCGIGSNPGIDGSRLLGAIAQ,
Protein, Cowpea mosaic virus (strain SB)
SEQ ID NO: 4
GPVCAEASDVYSPCMIASTPPAPFSDVTAVTFDLINGKITPVGDDNWNTH
IYNPPIMNVLRTAAWKSGTIHVQLNVRGAGVKRADWDGQVFVYLRQSMNP
ESYDARTFVISQPGSAMLNFSFDIIGPNSGFEFAESPWANQTTWYLECVA
TNPRQIQQFEVNMRFDPNFRVAGNILMPPFPLSTETPPL,
Protein, Bacteriophage QB
SEQ ID NO: 5
MAKLETVTLGNIGKDGKQTLVLNPRGVNPTNGVASLSQAGAVPALEKRVT
VSVSQPSRNRKNYKVQVKIQNPTACTANGSCDPSVTRQAYADVTFSFTQY
STDEERAFVRTELAALLASPLLIDAIDQLNPAY,
SEQ ID NO: 6
GHSHGKGCGK
Protein, Monkey
SEQ ID NO: 7
GHHHKPGLGE,
Protein, Artificial Sequence
SEQ ID NO: 8
CGSGRGHGHSHGKG,

1. A nanoparticle comprising a virus or virus like particle (VLP) and a S100A9 peptide epitope.

2. The nanoparticle of claim 1, wherein the virus is Cowpea mosaic virus (CPMV) and the virus like particle is Qβ capsid protein (CP).

3. The nanoparticle of claim 1, wherein the S100A9 peptide epitope comprises or consists of 101 [PGHHHKPGLG] 110 (human) (SEQ ID NO: 1) or 101 [RGHGHSHGKG] 110 (murine) (SEQ ID NO: 2).

4. The nanoparticle of claim 1, wherein the virus or VLP has an exposed lysine side chain.

5. The nanoparticle of claim 1, wherein the S100A9 epitope comprises a linker or a c-terminal cysteine, or optionally, is detectably labeled.

6. The nanoparticle of claim 5, wherein a N-hydroxysuccinimide (NHS) ester conjugates with the lysine side chain and a maleimide of a maleimide-polyethylene glycol₈ (SM(PEG)₈) conjugates with the c-terminal cysteine of the peptide.

7. The nanoparticle of claim 1, wherein the nanoparticle has an average diameter of from about 10 to about 50 nm.

8. (canceled)

9. A polynucleotide encoding the nanoparticle of claim 1.

10. A vector comprising the polynucleotide of claim 9.

11. An isolated host cell comprising the nanoparticle of claim 1.

12. A plurality of the nanoparticles of claim 1, wherein the nanoparticles are the same or different from each other.

13. A composition comprising the nanoparticle of claim 1 and a carrier.

14. (canceled)

15. A method for inducing an immune response in a subject in need thereof comprising administering to the subject the nanoparticle of claim 1.

16. A method for one or more of: inducing an immune response, treating cardiovascular disease and associated disorders, atherosclerosis, cancer and associated disorders, administering to a subject in need thereof with the nanoparticle of claim 1.

17-22. (canceled)

23. The method of claim 16, wherein the method treats a cancer expressing S100A9.

24-25. (canceled)

26. The method of claim 16, wherein the method treats cancer and the treatment comprises one or more of: inhibiting metastatic potential of the cancer; recurrence prevention, reduction in tumor size; a reduction in tumor burden, longer progression free survival and longer overall survival of the subject.

27. The method of any of claim 16, wherein the method treats CVD and associated disorders, wherein the method further comprises administering one or more of statins to reduce plasma cholesterol levels, angioplasty, lifestyle changes, beta blockers, nitrates, angiotensin-converting enzyme inhibitors, angiotensin-2 receptor blockers, calcium channel blockers or diuretics.

28. A kit comprising the nanoparticle of claim 1 and optional instructions for use.


SEQUENCE LISTING (13 sequences)

SEQ ID NO: 1 - protein, 10 aa, Homo sapiens
PGHHHKPGLG

SEQ ID NO: 2 - protein, 10 aa, Mus musculus
RGHGHSHGKG

SEQ ID NO: 3 - protein, 374 aa, Cowpea mosaic virus
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

SEQ ID NO: 4 - protein, 189 aa, Cowpea mosaic virus
GPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPL

SEQ ID NO: 5 - protein, 133 aa, Qubevirus durum
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 6 - protein, 10 aa, Mus musculus
GHSHGKGCGK

SEQ ID NO: 7 - protein, 10 aa, Macaca mulatta
GHHHKPGLGE

SEQ ID NO: 8 - protein, 14 aa, synthetic construct
CGSGRGHGHSHGKG

SEQ ID NO: 9 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 10 - protein, 113 aa, Mus musculus
MANKAPSQMERSITTIIDTFHQYSRKEGHPDTLSKKEFRQMVEAQLATFMKKEKRNEALINDIMEDLDTNQDNQLSFEECMMLMAKLIFACHEKLHENNPRGHGHSHGKGCGK

SEQ ID NO: 11 - protein, 5 aa, synthetic construct
GGGSC

SEQ ID NO: 12 - protein, 4 aa, synthetic construct
CGSG

SEQ ID NO: 13 - skipped